(19) United States
(12) Patent Application Publication
(10) Pub. No.: US 2021/0283091 A1
(43) Pub. Date: Sep. 16, 2021

(54) DECREASING IMMUNE ACTIVITY THROUGH MODULATION OF POSTCELLULAR SIGNALING FACTORS

(71) Applicant: FLAGSHIP PIONEERING INNOVATIONS V, INC., Cambridge, MA (US)

(72) Inventors: Anthony Michael Barsotti, New York, NY (US); Alexandra Masu Cantley, New York, NY (US); Jason Park, West Newton, MA (US)

(21) Appl. No.: 17/252,214

(22) PCT Filed: Jun. 14, 2019
(86) PCT No.: PCT/US2019/037373

§ 371 Date: Dec. 14, 2020

Related U.S. Application Data
(60) Provisional application No. 62/781,822, filed on Dec. 19, 2018.
(60) Provisional application No. 62/685,763, filed on Jun. 15, 2018.

(51) Int. Cl. A61K 31/245; A61K 33/04; A61K 31/45; A61K 31/095; A61K 31/137; A61K 31/277; A61K 31/353; A61K 31/407; A61K 31/122; A61K 31/381; A61K 31/355; A61K 31/202; A61K 31/05; A61K 31/085; A61K 31/499; A61K 38/12; A61K 31/16; A61K 31/4418; A61K 31/4412; A61K 49/00; A61K 31/517; A61K 31/437; A61K 31/03; A61K 31/185; A61K 31/357; A61K 31/40; A61K 31/405; A61K 31/366; A61K 31/47; A61K 31/22; A61K 31/505; A61K 31/44; A61K 31/655; A61K 33/243; A61K 38/51; A61K 33/00; A61K 33/20; A61K 33/26; A61K 31/455; A61K 31/198; A61P 37/06; A61P 35/00; G01N 33/50

(52) U.S. Cl. CPC A61K 31/245; G01N 33/505; A61K 31/45; A61K 31/095; A61K 31/137; A61K 31/277; A61K 31/353; A61K 31/407; A61K 31/122; A61K 31/381; A61K 31/355; A61K 31/202; A61K 31/05; A61K 31/085; A61K 31/499; A61K 38/12; A61K 31/16; A61K 31/4418; A61K 31/4412; A61K 49/0008; A61K 31/517; A61K 31/437; A61K 31/03; A61K 31/185; A61K 31/357; A61K 31/40; A61K 31/405; A61K 31/366; A61K 31/47; A61K 31/22; A61K 31/505; A61K 31/44; A61K 31/655; A61K 33/243; A61K 38/51; A61K 33/00; A61K 33/20; A61K 33/26; A61K 31/455; A61K 31/198; A61P 37/06; A61P 35/00; G01N 33/5026; G01N 33/5047; A61K 33/04

(57) ABSTRACT

The invention provides methods of decreasing immune response by inhibiting iron-dependent cellular disassembly. The decrease in immune response may be used, for example, for treatment of a disorder associated with iron-dependent cellular disassembly, including an autoimmune disorder, allergy, or an inflammatory disorder. The invention also provides screening assays for identification of compounds that inhibit iron-dependent cellular disassembly and are also immunoinhibitory agents.

### RELATED APPLICATIONS

This application claims priority to U.S. Provisional Patent Application No. 62/685,763 filed on Jun. 15, 2018, and U.S. Provisional Patent Application No. 62/781,822 filed on Dec. 19, 2018, the contents of each of which are incorporated by reference herein in their entirety.

### BACKGROUND

In multicellular organisms, cell death is a critical and active process that is believed to maintain tissue homeostasis and eliminate potentially harmful cells.

### SUMMARY OF THE INVENTION

In certain aspects, the disclosure relates to method of decreasing immune activity in a cell, tissue or subject, the method comprising administering to the cell, tissue or subject an agent that inhibits iron-dependent cellular disassembly in an amount sufficient to decrease the immune activity relative to a cell, tissue or subject that is not treated with the agent that inhibits iron-dependent cellular disassembly, wherein the agent that inhibits iron-dependent cellular disassembly comprises Ferrostatin-1 or a derivative or analog thereof.

In some embodiments, the Ferrostatin-1 derivative or analog thereof is represented by the following formula:

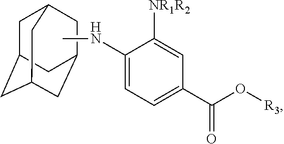

or an N-oxide, crystalline form, hydrate, or pharmaceutically acceptable salt thereof, wherein:

R₁ and R₂, are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, deuterium, C₁₋₄alkyl, CF₃, and combinations thereof; and

R₃ is independently selected from the group consisting of H, C₁₋₁₂aliphatic, C₁₋₆-alkyl-aryl and C₁₋₆-alkyl-heteroaryl;

with the proviso that:

when R₃ is ethyl, R₁ and R₂ cannot be both H, O, or

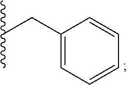 [SMILES: *Cc1ccccc1]

and

when R₃ is ethyl and at least one of R₁ or R₂ is H, R₁ or R₂ cannot be

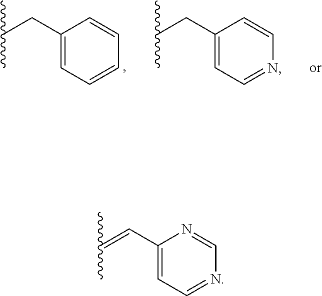

In some embodiments, the Ferrostatin-1 derivative or analog thereof is represented by the following formula:

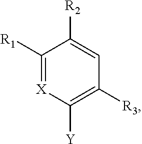

or a pharmaceutically acceptable salt, enantiomer, diastereomer thereof, wherein

X is CH or N;

Y is H, halo, or C₁₋₄alkyl;

R₁ is selected from the group consisting of H, halo, cycloalkyl, and NR₄R₅;

R₂ is selected from the group consisting of NR₆R₇ and NO2;

R₃ is selected from the group consisting of H,

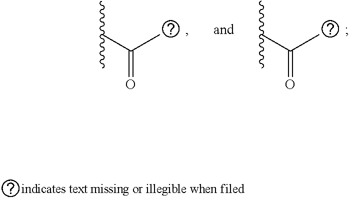

R₄ and R₅ are independently selected from the group consisting of H, C₁₋₁₂alkyl, C₃₋₁₂cycloalkyl, and aryl, wherein one or more of the ring carbons of the cycloalkyl are optionally substituted with one or more heteroatoms, and the cycloalkyl optionally comprises one or more pendant groups selected from the group consisting of H, F, NR₁₀R₁₁, Boc, COOR₁₂, and C₁₋₈alkyl;

R₆ and R₇ are independently selected from the group consisting of H, C₁₋₆alkyl, Boc, O, COOR₁₂,

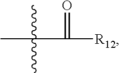

and C₁₋₃alkyl-aryl, wherein one or more of the ring carbons of the alkyl-aryl are optionally substituted with one or more nitrogen atoms, and the alkyl-aryl optionally comprises one or more pendant groups selected from the group consisting of H, halo, CN, NO₂, CM ether, C₁₋₄ ester, OCOOR₁₂, and C₁₋₈alkyl, which C₁₋₈alkyl is optionally further substituted with one or more halo;

R₈ and R₉ are independently selected from the group consisting of no atom, O, N, NHR₁₂, C₁₋₁₀ alkyl, and C₁₋₁₀ ether, wherein the alkyl and the ether are optionally substituted with NH₂, NHBoc, or C₃₋₁₂cycloalkyl, wherein one or more of the ring carbons of the cycloalkyl are optionally substituted with one or more heteroatoms;

R₁₀ and R₁₁ are independently selected from H and Boc; and

R₁₂ is a C₁₋₄alkyl optionally substituted with aryl,

with the proviso that:

when R₁ is H, R₃ cannot be

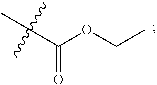 [SMILES: *C(=O)OCC]

when R₁ is

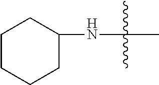 [SMILES: *NC1CCCCC1]

and R₂ is NH₂, R₃ cannot be

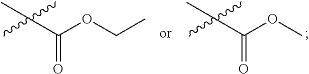

when R₁ is

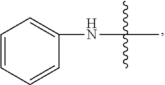 [SMILES: *Nc1ccccc1]

R₃ cannot be

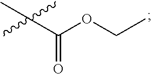 [SMILES: *C(=O)OCC]

when R₁ is

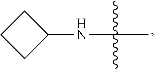 [SMILES: *NC1CCC1]

R₃ cannot be

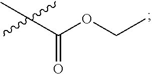 [SMILES: *C(=O)OCC]

when R₁ is Cl, X cannot be N, and

both R₁ and Y cannot be F.

In some embodiments, the subject is in need of decreased immune activity. In some embodiments, the agent that inhibits iron-dependent cellular disassembly is administered in an amount sufficient to decrease in the cell, tissue or subject one or more of: the level or activity of NFkB, the level or activity of interferon regulatory factor (IRF) or Stimulator of Interferon Genes (STING), the level or activity of macrophages, the level or activity of monocytes, the level or activity of dendritic cells, the level or activity of T cells, the level or activity of CD4+, CD8+ or CD3+ cells, and the level or activity of a pro-immune cytokine.

In certain aspects, the disclosure relates to a method of decreasing immune activity in a cell, tissue or subject, the method comprising administering to the cell, tissue or subject an agent that inhibits iron-dependent cellular disassembly in an amount sufficient to decrease the immune activity relative to a cell, tissue or subject that is not treated with the agent that inhibits iron-dependent cellular disassembly.

In one embodiment, the subject is in need of decreased immune activity.

In one embodiment, the agent that inhibits iron-dependent cellular disassembly is administered in an amount sufficient to decrease in the cell, tissue or subject one or more of: the level or activity of NFkB, the level or activity of IRF or STING, the level or activity of macrophages, the level or activity of monocytes, the level or activity of dendritic cells, the level or activity of T cells, the level or activity of CD4+, CD8+ or CD3+ cells, and the level or activity of a pro-immune cytokine.

In certain aspects, the disclosure relates to a method of decreasing the level or activity of NFkB in a cell, tissue or subject, comprising administering to the cell, tissue or subject an agent that inhibits iron-dependent cellular disassembly in an amount sufficient to decrease the level or activity of NFkB relative to a cell, tissue or subject that is not treated with the agent that inhibits iron-dependent cellular disassembly.

In one embodiment, the subject is in need of a decreased level or activity of NFkB.

In one embodiment, the level or activity of NFkB is decreased by at least 10%, 20%, 30%, 40%, 50%, 60%, 70%, 80%, 90% or 99%, or by at least 2-fold, 4-fold, 6-fold, 8-fold, or 10-fold relative to a cell, tissue or subject that is not treated with the agent that inhibits iron-dependent cellular disassembly.

In certain aspects, the disclosure relates to a method of decreasing the level or activity of IRF or STING in a cell, tissue or subject, comprising administering to the cell, tissue or subject an agent that inhibits iron-dependent cellular disassembly in an amount sufficient to decrease the level or activity of IRF or STING relative to a cell, tissue or subject that is not treated with the agent that inhibits iron-dependent cellular disassembly.

In one embodiment, the subject is in need of a decreased level or activity of IRF or STING.

In one embodiment, the level or activity of IRF or STING is increased by at least 10%, 20%, 30%, 40%, 50%, 60%, 70%, 80%, 90% or 99%, or by at least 2-fold, 4-fold, 6-fold, 8-fold, or 10-fold relative to a cell, tissue or subject that is not treated with the agent that inhibits iron-dependent cellular disassembly.

In certain aspects, the disclosure relates to a method of decreasing the level or activity of macrophages, monocytes, dendritic cells or T cells in a tissue or subject, comprising administering to the tissue or subject an agent that inhibits iron-dependent cellular disassembly in an amount sufficient to increase the level or activity of macrophages, monocytes, dendritic cells or T cells relative to a tissue or subject that is not treated with the agent that inhibits iron-dependent cellular disassembly.

In one embodiment, the subject is in need of a decreased level or activity of macrophages, monocytes, dendritic cells or T cells.

In one embodiment, the level or activity of macrophages, monocytes, dendritic cells, or T cells is decreased by at least 10%, 20%, 30%, 40%, 50%, 60%, 70%, 80%, 90% or 99%, or by at least 2-fold, 4-fold, 6-fold, 8-fold, or 10-fold relative to a tissue or subject that is not treated with the agent that inhibits iron-dependent cellular disassembly.

In certain aspects, the disclosure relates to a method of decreasing the level or activity of CD4+, CD8+, or CD3+ cells in a tissue or subject, comprising administering to the subject an agent that inhibits iron-dependent cellular disassembly in an amount sufficient to decrease the level or activity of CD4+, CD8+, or CD3+ cells relative to a tissue or subject that is not treated with the agent that inhibits iron-dependent cellular disassembly.

In one embodiment, the subject is in need of a decreased level or activity of CD4+, CD8+, or CD3+ cells.

In one embodiment, the level or activity of CD4+, CD8+, or CD3+ cells is decreased by at least 10%, 20%, 30%, 40%, 50%, 60%, 70%, 80%, 90% or 99%, or by at least 2-fold, 4-fold, 6-fold, 8-fold, or 10-fold relative to a tissue or subject that is not treated with the agent that inhibits iron-dependent cellular disassembly.

In certain aspects, the disclosure relates to a method of decreasing the level or activity of a pro-immune cytokine in a cell, tissue or subject, comprising administering to the cell, tissue or subject an agent that inhibits iron-dependent cellular disassembly in an amount sufficient to decrease the level or activity of the pro-immune cytokine relative to a cell, tissue or subject that is not treated with the agent that inhibits iron-dependent cellular disassembly.

In one embodiment, the subject is in need of a decreased level or activity of a pro-immune cytokine.

In one embodiment, the level or activity of the pro-immune cytokine is decreased by at least 10%, 20%, 30%, 40%, 50%, 60%, 70%, 80%, 90% or 99%, or by at least 2-fold, 4-fold, 6-fold, 8-fold, or 10-fold relative to a cell, tissue or subject that is not treated with the agent that inhibits iron-dependent cellular disassembly.

In one embodiment, the pro-immune cytokine is selected from IFN-α, IL-1, IL-12, IL-18, IL-2, IL-15, IL-4, IL-6, TNF-α, IL-17 and GMCSF.

In one embodiment, the method further includes, before the administration, evaluating the cell, tissue or subject for one or more of: the level or activity of NFkB; the level or activity of macrophages; the level or activity of monocytes; the level or activity of dendritic cells; the level or activity of CD4+ cells, CD8+ cells, or CD3+ cells; the level or activity of T cells; and the level or activity of a pro-immune cytokine.

In one embodiment, the method further includes, after the administration, evaluating the cell, tissue or subject for one or more of: the level or activity of NFkB; the level or activity of macrophages; the level or activity of monocytes; the level or activity of dendritic cells; the level or activity of CD4+ cells, CD8+ cells or CD3+ cells; the level or activity of T cells; and the level or activity of a pro-immune cytokine.

In embodiments, the pro-immune cytokine is selected from IFN-α, IL-1, IL-12, IL-18, IL-2, IL-15, IL-4, IL-6, TNF-α, IL-17 and GMCSF.

In one embodiment, the subject has an inflammatory disease or condition.

In one embodiment, the inflammatory disease or condition is selected from the group consisting of inflammation, acute organ injury, tissue damage, sepsis, atherosclerosis, a neurodegenerative disorder, and an immune-related disease or condition.

In one embodiment, the inflammation is selected from sterile inflammation, chronic inflammation, and acute inflammation in response to disease or injury.

In one embodiment, the immune-related disease or condition is an autoimmune disease.

In one embodiment, the autoimmune disease is selected from systemic lupus erythematosus (SLE), rheumatoid arthritis, Type I diabetes, Type II diabetes, multiple sclerosis (MS), amyotrophic lateral sclerosis (ALS), graft-vs-host disease (GVHD), psoriasis, and ulcerative colitis.

In one embodiment, the immune-related disease or condition is an allergy or asthma.

In one embodiment, the immune-related disease or condition is an autoinflammatory condition.

In certain aspects, the disclosure relates to a method of treating a subject in need of decreased immune activity, the method comprising administering to the subject an agent that inhibits iron-dependent cellular disassembly in an amount sufficient to decrease the immune activity in the subject.

In one embodiment, the subject has a disorder associated with iron-dependent cellular disassembly.

In one embodiment, the subject has a disorder in which iron-dependent cellular disassembly is detrimental.

In one embodiment, the method further comprises evaluating the subject for iron-dependent cellular disassembly.

In one embodiment, the subject has an inflammatory disease or condition.

In one embodiment, the inflammatory disease or condition is selected from the group consisting of inflammation, acute organ injury, tissue damage, sepsis, atherosclerosis, a neurodegenerative disorder, and an immune-related disease or condition.

In one embodiment, the inflammation is selected from sterile inflammation, chronic inflammation, and acute inflammation in response to disease or injury.

In one embodiment, the immune-related disease or condition is an autoimmune disease.

In one embodiment, the autoimmune disease is selected from systemic lupus erythematosus (SLE), rheumatoid arthritis, Type I diabetes, Type II diabetes, multiple sclerosis (MS), amyotrophic lateral sclerosis (ALS), graft-vs-host disease (GVHD), psoriasis, and ulcerative colitis.

In one embodiment, the immune-related disease or condition is an allergy or asthma.

In one embodiment, the immune-related disease or condition is an autoinflammatory condition.

In one embodiment, the agent that inhibits iron-dependent cellular disassembly is selected from an inhibitor of lipid peroxidation, an inhibitor of glutaminolysis, an inhibitor of lipoxygenase, an inhibitor of cysteine dioxygenase 1 (CD01), and an inhibitor of DPP4.

In one embodiment, the agent that inhibits iron-dependent cellular disassembly is selected from cycloheximide, beta-mercaptoethanol, dopamine, and selenium.

In one embodiment, the inhibitor of lipoxygenase is an inhibitor of arachidonate lipoxygenase (ALOX).

In one embodiment, the inhibitor of lipoxygenase is selected from the group consisting of CDC, baicalein, PD-146176, AA-861, and zileuton.

In one embodiment, the inhibitor of lipid peroxidation is selected from vitamin E, alpha-tocopherol, trolox, tocotrienols, deuterated polyunsaturated fatty acids (D-PUFAs), butylated hydroxytoluene, butylated hydroxyanisole, Ferrostatin-1 or a derivative or analog thereof, Liproxstatin-1 or a derivative or analog thereof, Coenzyme Q10, idebenone, XJB-5-131, deferoxamine, cyclipirox, and deferiprone.

In a particular embodiment, the agent that inhibits iron-dependent cellular disassembly is Ferrostatin-1.

In one embodiment, Ferrostatin-1 derivative or analog thereof is represented by the following formula:

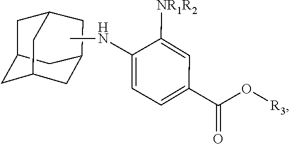

or an N-oxide, crystalline form, hydrate, or pharmaceutically acceptable salt thereof, wherein:

R₁ and R₂, are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, deuterium, C₁₋₄alkyl, CF₃, and combinations thereof; and

R₃ is independently selected from the group consisting of H, C₁₋₁₂aliphatic, C₁₋₆-alkyl-aryl and C₁₋₆-alkyl-heteroaryl;

with the proviso that:

when R₃ is ethyl, R₁ and R₂ cannot be both H, O, or

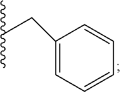 [SMILES: *Cc1ccccc1]

and

when R₃ is ethyl and at least one of R₁ or R₂ is H, R₁ or R₂ cannot be

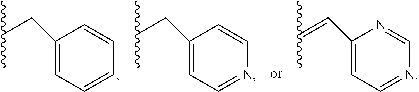

In one embodiment, Ferrostatin-1 derivative or analog thereof is represented by the following formula:

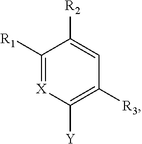

or a pharmaceutically acceptable salt, enantiomer, diastereomer thereof, wherein

X is CH or N;

Y is H, halo, or C₁₋₄alkyl;

R₁ is selected from the group consisting of H, halo, cycloalkyl, and NR₄R₅;

R₂ is selected from the group consisting of NR₆R₇ and NO2;

R₃ is selected from the group consisting of H,

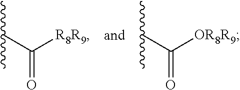

R₄ and R₅ are independently selected from the group consisting of H, C₁₋₁₂alkyl, C₃₋₁₂cycloalkyl, and aryl, wherein one or more of the ring carbons of the cycloalkyl are optionally substituted with one or more heteroatoms, and the cycloalkyl optionally comprises one or more pendant groups selected from the group consisting of H, F, NR₁₀R₁₁, Boc, COOR₁₂, and C₁₋₈alkyl;

R₆ and R₇ are independently selected from the group consisting of H, C₁₋₆alkyl, Boc, O, COOR₁₂,

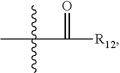

and C₁₋₃alkyl-aryl, wherein one or more of the ring carbons of the alkyl-aryl are optionally substituted with one or more nitrogen atoms, and the alkyl-aryl optionally comprises one or more pendant groups selected from the group consisting of H, halo, CN, NO₂, C₁₋₄ ether, C₁₋₄ ester, OCOOR₁₂, and C₁₋₈alkyl, which C₁₋₈alkyl is optionally further substituted with one or more halo;

R₈ and R₉ are independently selected from the group consisting of no atom, O, N, NHR₁₂, C₁₋₁₀ alkyl, and C₁₋₁₀ ether, wherein the alkyl and the ether are optionally substituted with NH₂, NHBOC, or C₃₋₁₂cycloalkyl, wherein one or more of the ring carbons of the cycloalkyl are optionally substituted with one or more heteroatoms;

R₁₀ and R₁₁ are independently selected from H and Boc; and

R₁₂ is a C₁₋₄alkyl optionally substituted with aryl,

with the proviso that:

when R₁ is H, R₃ cannot be

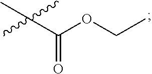 [SMILES: *C(=O)OCC]

when R₁ is

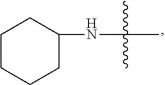 [SMILES: *NC1CCCCC1]

and R₂ is NH₂, R₃ cannot be

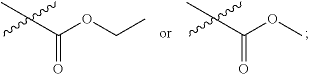

when R₁ is

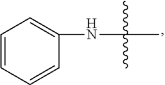 [SMILES: *Nc1ccccc1]

R₃ cannot be

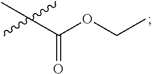 [SMILES: *C(=O)OCC]

when R₁ is

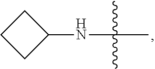 [SMILES: *NC1CCC1]

R₃ cannot be

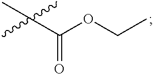 [SMILES: *C(=O)OCC]

when R₁ is Cl, X cannot be N, and

both R₁ and Y cannot be F.

In a particular embodiment, the agent that inhibits iron-dependent cellular disassembly is

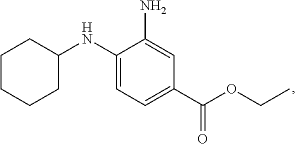 [SMILES: CCOC(=O)c1ccc(NC2CCCCC2)c(N)c1]

or a pharmaceutically acceptable salt thereof.

In a particular embodiment, the agent that inhibits iron-dependent cellular disassembly is

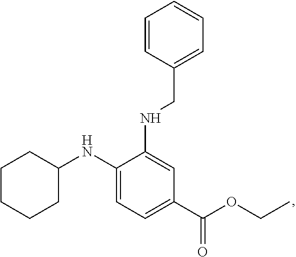 [SMILES: CCOC(=O)c1ccc(NC2CCCCC2)c(NCc2ccccc2)c1]

or a pharmaceutically acceptable salt thereof.

In a particular embodiment, the agent that inhibits iron-dependent cellular disassembly is

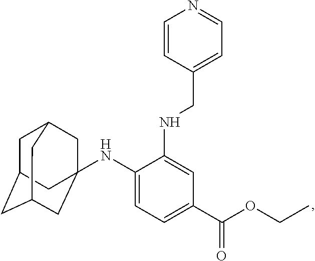 [SMILES: CCOC(=O)c1ccc(NC23CC4CC(CC(C4)C2)C3)c(NCc2ccncc2)c1]

or a pharmaceutically acceptable salt thereof.

In one embodiment, the inhibitor of DPP4 is selected from vildagliptin, alogliptin, and linagliptin.

In certain aspects, the disclosure relates to a method of screening for an immunoinhibitory agent, the method comprising:

(a) providing a plurality of test agents (e.g., a library of test agents);
(b) evaluating each of the plurality of test agents for the ability to inhibit iron-dependent cellular disassembly;
(c) selecting as a candidate immunoinhibitory agent a test agent that inhibits iron-dependent cellular disassembly; and
(d) evaluating the candidate immunoinhibitory agent for the ability to decrease an immune response.

In one embodiment, the evaluating step (b) comprises contacting cells, tissue, or a subject with each of the plurality of test agents.

In one embodiment, the evaluating step (b) comprises contacting cells, tissue, or a subject with an agent that induces iron-dependent cellular disassembly.

In one embodiment, the subject is an animal.

In one embodiment, the evaluating step (b) further comprises measuring the level or activity of a marker selected from the group consisting of lipid peroxidation, reactive oxygen species (ROS), isoprostanes, malondialdehyde (MDA), iron, glutathione peroxidase 4 (GPX4), prostaglandin-endoperoxide synthase 2 (PTGS2), cyclooxygenase-2 (COX-2), and glutathione (GSH) in the cells or tissue contacted with the test agent.

In one embodiment, the evaluating step (b) further comprises comparing the level or activity of the marker in the cells, tissue or subject contacted with the test agent to the level or activity of the marker in a control cell, control tissue or control subject that has not been contacted with the test agent.

In one embodiment, the control cell, control tissue or control subject has been contacted with an agent that induces ferroptosis.

In one embodiment, a decrease in the level or activity of a marker selected from the group consisting of lipid peroxidation, isoprostanes, reactive oxygen species (ROS), iron, PTGS2 and COX-2 in the cells, tissue or subject contacted with the test agent relative to the level or activity of the marker in a control cell, control tissue or control subject that has not been contacted with the test agent indicates that the test agent is an agent that inhibits iron-dependent cellular disassembly.

In one embodiment, an increase in the level or activity of a marker selected from the group consisting of GPX4, MDA and GSH in the cells, tissue or subject contacted with the test agent relative to the level or activity of the marker in a control cell, control tissue or control subject that has not been contacted with the test agent indicates that the test agent is an agent that inhibits iron-dependent cellular disassembly.

In one embodiment, evaluating the candidate immunoinhibitory agent comprises culturing an immune cell together with cells contacted with the selected candidate immunoinhibitory agent or exposing an immune cell to postcellular signaling factors produced by cells contacted with the selected candidate immunoinhibitory agent and measuring the level or activity of NFκB, IRF or STING in the immune cell.

In one embodiment, the immune cell is a THP-1 cell.

In one embodiment, evaluating the candidate immunoinhibitory agent comprises culturing T cells together with cells contacted with the selected candidate immunoinhibitory agent or exposing T cells to postcellular signaling factors produced by cells contacted with the selected candidate immunoinhibitory agent and measuring the activation and proliferation of the T cells.

In one embodiment, evaluating the candidate immunoinhibitory agent further comprises comparing the level or activity of NFκB, IRF or STING in the immune cell cultured with cells contacted with the selected candidate immunoinhibitory agent or exposed to postcellular signaling factors produced by cells contacted with the selected candidate immunoinhibitory agent with the level or activity of NFκB, IRF or STING in control immune cells that have not been contacted with the selected candidate immunoinhibitory agent and have not been exposed to postcellular signaling factors produced by cells contacted with the selected candidate immunoinhibitory agent.

In one embodiment, the cells contacted with the selected candidate immunoinhibitory agent have also been contacted with an agent that induces iron-dependent cellular disassembly.

In one embodiment, the agent that induces iron-dependent cellular disassembly is selected from the group consisting of an inhibitor of antiporter system Xc⁻, an inhibitor of GPX4, and a statin.

In one embodiment, the inhibitor of antiporter system Xc⁻ is erastin or a derivative or analog thereof.

In one embodiment, the analog of erastin is PE or IKE.

In one embodiment, the inhibitor of GPX4 is selected from the group consisting of (1S,3R)-RSL3 or a derivative or analog thereof, ML162, DPI compound 7, DPI compound 10, DPI compound 12, DPI compound 13, DPI compound 17, DPI compound 18, DPI compound 19, FIN56, and FIN02.

In one embodiment, the statin is selected from the group consisting of atorvastatin, fluvastatin, lovastatin, pitavastatin, pravastatin, rosuvastatin, cerivastatin and simvastatin.

In one embodiment, the agent that induces iron-dependent cellular disassembly is selected from the group consisting of sorafenib or a derivative or analog thereof, sulfasalazine, glutamate, BSO, DPI2, cisplatin, cysteinase, silica based nanoparticles, CCI4, ferric ammonium citrate, trigonelline and brusatol.

### BRIEF DESCRIPTION OF THE DRAWINGS

FIG. 1A shows HT1080 fibrosarcoma cells treated with various concentrations of erastin. FIG. 1B shows NFkB activity in THP1 monocytes co-cultured with HT1080 cells treated with erastin. Error bars represent standard deviation among three replicates.

FIG. 1C shows HT1080 fibrosarcoma cells treated with DMSO or various concentrations of erastin (ERAS) or the erastin analogs piperazine erastin (PE) or imidazole ketoerastin (IKE). The DMSO control is on the far left. The erastin or erastin analog concentrations increase from left to right and are the same as those shown in FIG. 1A.

FIG. 1D shows NFkB activity in THP1 monocytes co-cultured with HT1080 cells treated with erastin (ERAS) or the erastin analogs piperazine erastin (PE) or imidazole ketoerastin (IKE). The DMSO control is on the far left. The erastin or erastin analog concentrations increase from left to right and are the same as those shown in FIG. 1B. Error bars represent standard deviation among three replicates.

FIG. 2A shows pancreatic cancer cells (PANC1) treated with various concentrations of erastin. FIG. 2B shows NFkB activity in THP1 monocytes co-cultured with PANC1 cells treated with erastin.

FIG. 3A shows renal cell carcinoma cells (Caki-1) treated with various concentrations of erastin. FIG. 3B shows NFkB activity in THP1 monocytes co-cultured with Caki-1 cells treated with erastin.

FIG. 4A shows renal cell carcinoma cells (Caki-1) treated with various concentrations of RSL3. FIG. 4B shows NFkB activity in THP1 monocytes co-cultured with Caki-1 cells treated with RSL3.

FIG. 5A shows Jurkat T cell leukemia cells treated with various concentrations of RSL3. FIG. 5B shows NFkB activity in THP1 monocytes co-cultured with Jurkat cells treated with RSL3.

FIG. 6A shows A20 B-cell leukemia cells treated with various concentrations of RSL3. FIG. 6B shows NFkB activity in THP1 monocytes co-cultured with A20 cells treated with RSL3. FIG. 6C shows IRF activity in THP1 monocytes co-cultured with A20 cells treated with RSL3.

FIG. 7A shows the viability of HT1080 fibrosarcoma cells treated with various concentrations of Erastin alone or in combination with a ferroptosis inhibitor (Ferrostatin-1, Liprox statin-1 or Trolox).

FIG. 7B shows NFkB activity in THP1 monocytes co-cultured with HT1080 fibrosarcoma cells treated with Erastin alone or in combination with a ferroptosis inhibitor (Ferrostatin-1, Liprox statin-1 or Trolox).

FIG. 8A shows the viability of HT1080 fibrosarcoma cells treated with various concentrations of Erastin alone or in combination with a ferroptosis inhibitor (Ferrostatin-1, β-Mercaptoethanol, or Deferoxamine).

FIG. 8B shows NFkB activity in THP1 monocytes co-cultured with HT1080 fibrosarcoma cells treated with Erastin alone or in combination with a ferroptosis inhibitor (Ferrostatin-1, β-Mercaptoethanol, or Deferoxamine).

FIG. 9A shows the viability of HT1080 fibrosarcoma cells treated with various concentrations of Erastin in combination with an siRNA control (siControl) or an siRNA directed to the ACSL4 gene (siACSL4).

FIG. 9B shows the viability of H1080 fibrosarcoma cells treated with DMSO or Erastin in combination with an siRNA control (siControl), an siRNA directed to the ACSL4 gene (siACSL4), or an siRNA directed to the CARS gene (siCARS).

FIG. 9C shows the fold change in NFkB activity in THP1 monocytes co-cultured with HT1080 fibrosarcoma cells treated with DMSO or Erastin in combination with an siRNA control (siControl), an siRNA directed to the ACSL4 gene (siACSL4), or an siRNA directed to the CARS gene (siCARS).

FIG. 10A shows the viability of A20 lymphoma cells treated with DMSO or various concentrations of RSL3 alone or in combination with Ferrostatin-1.

FIG. 10B shows NFkB activity in THP1 monocytes co-cultured with A20 lymphoma cells treated with DMSO or various concentrations of RSL3 alone or in combination with Ferrostatin-1.

FIG. 11A shows the viability of A20 lymphoma cells treated with DMSO or various concentrations of ML162 alone or in combination with Ferrostatin-1.

FIG. 11B shows NFkB activity in THP1 monocytes co-cultured with A20 lymphoma cells treated with DMSO or various concentrations of ML162 alone or in combination with Ferrostatin-1.

FIG. 12A shows the viability of A20 lymphoma cells treated with DMSO or various concentrations of ML210 alone or in combination with Ferrostatin-1.

FIG. 12B shows NFkB activity in THP1 monocytes co-cultured with A20 lymphoma cells treated with DMSO or various concentrations of ML210 alone or in combination with Ferrostatin-1.

FIG. 13A shows the viability of Caki-1 renal carcinoma cells treated with DMSO or various concentrations of RSL3 alone or in combination with Ferrostatin-1.

FIG. 13B shows NFkB activity in THP1 monocytes co-cultured with Caki-1 renal carcinoma cells treated with DMSO or various concentrations of RSL3 alone or in combination with Ferrostatin-1.

FIG. 14A shows the viability of Caki-1 renal carcinoma cells treated with DMSO or various concentrations of ML162 alone or in combination with Ferrostatin-1.

FIG. 14B shows NFkB activity in THP1 monocytes co-cultured with Caki-1 renal carcinoma cells treated with DMSO or various concentrations of ML162 alone or in combination with Ferrostatin-1.

### DETAILED DESCRIPTION

The present disclosure relates to methods of decreasing immune activity in a cell, tissue or subject comprising administering to the cell, tissue or subject an agent that inhibits iron-dependent cellular disassembly. Applicants have surprisingly shown that induction of iron-dependent cellular disassembly (e.g. ferroptosis) increases immune response as evidenced by increases in NFKB and IRF activity in immune cells. Based on these results, inhibition of iron-dependent cellular disassembly is expected to decrease immune response by preventing the induction of immunostimulatory activity caused by iron-dependent cellular disassembly. Accordingly, administration of an agent that inhibits iron-dependent cellular disassembly may be used to treat disorders that would benefit from decreased immune activity, such as inflammatory diseases.

### I. Definitions

The terms “administer”, “administering” or “administration” include any method of delivery of a pharmaceutical composition or agent into a subject's system or to a particular region in or on a subject.

“Ferroptosis”, as used herein, refers to a process of regulated cell death that is iron dependent and involves the production of reactive oxygen species.

“Cellular disassembly” refers to a dynamic process that reorders and disseminates the material within a cell and may ultimately result in cell death. The cellular disassembly process includes the production and release from the cell of postcellular signaling factors.

As used herein, the terms “increasing” (or “activating”) and “decreasing” refer to modulating resulting in, respectively, greater or lesser amounts, function or activity of a parameter relative to a reference. For example, subsequent to administration of a preparation described herein, a parameter (e.g., NFkB activity, activation of macrophages or T cells) may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration. Generally, the metric is measured subsequent to administration at a time that the administration has had the recited effect, e.g., at least one day, one week, one month, 3 months, 6 months, after a treatment regimen has begun. Similarly, pre-clinical parameters (such NFkB activity of cells in vitro, and/or activity and proliferation of T cells) may be decreased by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration.

“Postcellular signaling factors” are molecules and cell fragments produced by a cell undergoing cellular disassembly (e.g., iron-dependent cellular disassembly) that are ultimately released from the cell and influence the biological activity of other cells. Postcellular signaling factors can include proteins, peptides, carbohydrates, lipids, nucleic acids, small molecules, and cell fragments (e.g. vesicles and cell membrane fragments).

A “subject” to be treated by the methods of the invention can mean either a human or non-human animal, preferably a mammal, more preferably a human. In certain embodiments, a subject has a detectable or diagnosed autoimmune disorder prior to initiation of treatments using the methods of the invention. In certain embodiments, a subject has a detectable or diagnosed allergy prior to initiation of treatments using the methods of the invention.

“Therapeutically effective amount” means the amount of a compound that, when administered to a patient for treating a disease, is sufficient to effect such treatment for the disease. When administered for preventing a disease, the amount is sufficient to avoid or delay onset of the disease. The “therapeutically effective amount” will vary depending on the compound, the disease and its severity and the age, weight, etc., of the patient to be treated. A therapeutically effective amount need not be curative. A therapeutically effective amount need not prevent a disease or condition from ever occurring. Instead a therapeutically effective amount is an amount that will at least delay or reduce the onset, severity, or progression of a disease or condition.

As used herein, “treatment”, “treating” and cognates thereof refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy).

### II. Iron-Dependent Cellular Disassembly

Cellular disassembly is a dynamic process that re-orders and disseminates the material within a cell, and which results in the production and release of postcellular signaling factors, or “effectors”, that can have a profound effect on the biological activity of other cells. Cellular disassembly occurs during the process of regulated cell death and is controlled by multiple molecular mechanisms. Different types of cellular disassembly result in the production of different postcellular signaling factors and thereby mediate different biological effects. For example, Applicants have surprisingly shown that induction of an iron-dependent cellular disassembly can increase immune response as evidenced by increases in NFKB and IRF activity in immune cells. These results suggest that inhibition of iron-dependent cellular disassembly would decrease immune response by preventing the induction of immunostimulatory activity caused by iron-dependent cellular disassembly.

In some embodiments, the iron-dependent cellular disassembly is ferroptosis. Ferroptosis is a process of regulated cell death involving the production of iron-dependent reactive oxygen species (ROS). In some embodiments, ferroptosis involves the iron-dependent accumulation of lipid hydroperoxides to lethal levels. The sensitivity to ferroptosis is tightly linked to numerous biological processes, including amino acid, iron, and polyunsaturated fatty acid metabolism, and the biosynthesis of glutathione, phospholipids, NADPH, and Coenzyme Q10. Ferroptosis involves metabolic dysfunction that results in the production of both cytosolic and lipid ROS, independent of mitochondria but dependent on NADPH oxidases in some cell contexts (Dixon et al., 2012, Cell 149(5): 1060-72).

Agents that Inhibit Iron-Dependent Cellular Disassembly

A broad range of agents that inhibit iron-dependent cellular disassembly, e.g., ferroptosis, are known in the art, and are useful in the various methods provided by the present invention.

Iron-dependent cellular disassembly (e.g. ferroptosis) may involve iron-dependent accumulation of lipid hydroperoxides to lethal levels, resulting in cell death. Accordingly, in one embodiment, the agent that inhibits iron-dependent cellular disassembly is an inhibitor of lipid peroxidation. Several inhibitors of lipid peroxidation are known in the art, including vitamin E, alpha-tocopherol, trolox, tocotrienols, deuterated polyunsaturated fatty acids (D-PUFAs), butylated hydroxytoluene, butylated hydroxyanisole, Ferrostatin-1 or a derivative or analog thereof, Fiproxstatin-1 or a derivative or analog thereof, Coenzyme Q10, idebenone, XJB-5-131, deferoxamine, deferoxamine mesylate, desferrioxamine, cyclipirox, and deferiprone. In a particular embodiment, the inhibitor of iron-dependent cellular disassembly (e.g. an inhibitor of lipid peroxidation) is an iron chelator, e.g. deferoxamine, deferoxamine mesylate, desferrioxamine, cyclipirox or deferiprone.

In a particular embodiment, the agent that inhibits iron-dependent cellular disassembly is Ferrostatin-1 or a derivative or analog thereof.

In one embodiment, Ferrostatin-1 or a derivative or analog thereof is represented by the following formula:

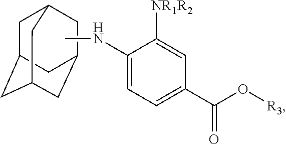

or an N-oxide, crystalline form, hydrate, or pharmaceutically acceptable salt thereof, wherein:

R₁ and R₂, are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, deuterium, C₁₋₄alkyl, CF₃, and combinations thereof; and

R₃ is independently selected from the group consisting of H, C₁₋₁₂aliphatic, C₁₋₆-alkyl-aryl and C₁₋₆-alkyl-heteroaryl;

with the proviso that:

when R₃ is ethyl, R₁ and R₂ cannot be both H, O, or

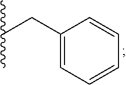 [SMILES: *Cc1ccccc1]

and

when R₃ is ethyl and at least one of R₁ or R₂ is H, R₁ or R₂ cannot be

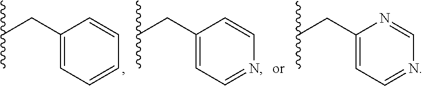

In one embodiment, Ferrostatin-1 or a derivative or analog thereof is represented by the following formula:

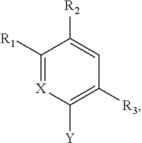

or a pharmaceutically acceptable salt, enantiomer, diastereomer thereof, wherein

X is CH or N;

Y is H, halo, or C₁₋₄alkyl;

R₁ is selected from the group consisting of H, halo, cycloalkyl, and NR₄R₅;

R₂ is selected from the group consisting of NR₆R₇ and NO2;

R₃ is selected from the group consisting of H,

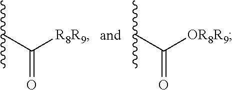

R₄ and R₅ are independently selected from the group consisting of H, C₁₋₁₂alkyl, C₃₋₁₂cycloalkyl, and aryl, wherein one or more of the ring carbons of the cycloalkyl are optionally substituted with one or more heteroatoms, and the cycloalkyl optionally comprises one or more pendant groups selected from the group consisting of H, F, NR₁₀R₁₁, Boc, COOR₁₂, and C₁₋₈alkyl;

R₆ and R₇ are independently selected from the group consisting of H, C₁₋₆alkyl, Boc, O, COOR₁₂,

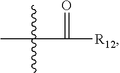

and C₁₋₃alkyl-aryl, wherein one or more of the ring carbons of the alkyl-aryl are optionally substituted with one or more nitrogen atoms, and the alkyl-aryl optionally comprises one or more pendant groups selected from the group consisting of H, halo, CN, NO₂, C₁₋₄ ether, C₁₋₄ ester, OCOOR₁₂, and C₁₋₈alkyl, which C₁₋₈alkyl is optionally further substituted with one or more halo;

R₈ and R₉ are independently selected from the group consisting of no atom, O, N, NHR₁₂, C₁₋₁₀ alkyl, and C₁₋₁₀ ether, wherein the alkyl and the ether are optionally substituted with NH₂, NHBOC, or C₃₋₁₂cycloalkyl, wherein one or more of the ring carbons of the cycloalkyl are optionally substituted with one or more heteroatoms;

R₁₀ and R₁₁ are independently selected from H and Boc; and

R₁₂ is a C₁₋₄alkyl optionally substituted with aryl,

with the proviso that:

when R₁ is H, R₃ cannot be

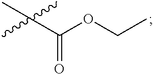 [SMILES: *C(=O)OCC]

when R₁ is

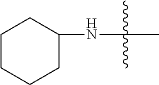 [SMILES: *NC1CCCCC1]

and R₂ is NH₂, R₃ cannot be

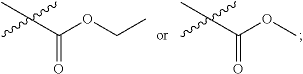

when R₁ is

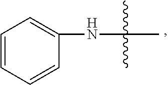 [SMILES: *Nc1ccccc1]

R₃ cannot be

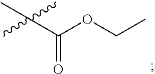 [SMILES: *C(=O)OCC]

when R₁ is

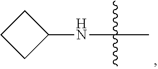 [SMILES: *NC1CCC1]

R₃ cannot be

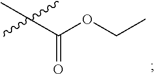 [SMILES: *C(=O)OCC]

when R₁ is Cl, X cannot be N, and

both R₁ and Y cannot be F.

In a particular embodiment, the agent that inhibits iron-dependent cellular disassembly is

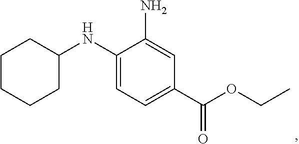 [SMILES: CCOC(=O)c1ccc(NC2CCCCC2)c(N)c1]

or a pharmaceutically acceptable salt thereof.

In a particular embodiment, the agent that inhibits iron-dependent cellular disassembly is

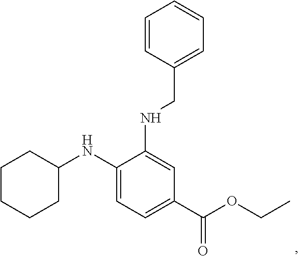 [SMILES: CCOC(=O)c1ccc(NC2CCCCC2)c(NCc2ccccc2)c1]

or a pharmaceutically acceptable salt thereof.

In a particular embodiment, the agent that inhibits iron-dependent cellular disassembly is

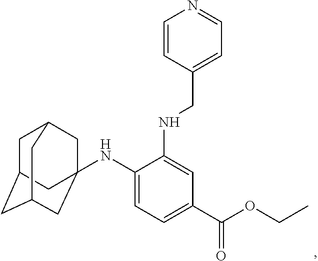 [SMILES: CCOC(=O)c1ccc(NC23CC4CC(CC(C4)C2)C3)c(NCc2ccncc2)c1]

or a pharmaceutically acceptable salt thereof.

Additional Ferrostatin-1 derivatives or analogs are described, for example in
WO2015/084749, WO2013/152039, WO2016/191520,
JACS (2014), 136(12), 4551-4556 doi: 10.1021/ja411006a,
J. Med. Chem. (2016), 59(5), 2041-2053, doi: 10.1021/acs.jmedchem.5b01641,
Bioorg. & Med. Chem. Lett. (2015), 25(22), 5315-5320, doi: 10.1016/j.bmcl.2015.09.041, and
J. Bio. Chem. (2009), 284(23), 15517-15529, doi: 10.1074/jbc.M808889200, each of which is incorporated by reference herein in its entirety.

Other molecules involved in iron-dependent cellular disassembly may also be used to inhibit this process. As an inducer of ferroptosis, erastin suppresses the glutamate/cystine antiporter (system Xc−), thereby inhibiting cellular cystine uptake and depleting glutathione (GSH), a primary cellular antioxidant that maintains the redox balance and defends against oxidative stress. The lipid repair enzyme glutathione peroxidase 4 (GPX4) negative regulates ferroptosis by limiting lipid hydroperoxide levels. GPX4 uses GSH to repair lipids and reduce lipid hydroperoxides to nontoxic alcohols. A decreased GSH level inactivates GPX4. Subsequently, cells fail to protect against toxic L-ROS, finally incurring ferroptosis. Thus GPX4 or GPX4 agonists may also be used as inhibitors of iron-dependent cellular disassembly.

Human cysteine dioxygenase 1 (CDO1), a non-heme iron metalloenzyme, transforms cysteine to taurine by catalyzing the oxidation of cysteine to its sulfinic acid. In addition to transforming into taurine, cellular cysteine is also an indispensable substrate for the synthesis of GSH, which consists of glutamate, cysteine, and glycine. A deficiency of cellular cysteine decreases GSH synthesis and impairs cellular antioxidant capacity, which ultimately results in enhanced ROS levels and the induction of ferroptosis. Silencing of CDO1 has been shown to inhibit erastin-induced ferroptosis both in vitro and in vivo. See Hao et al., 2017, Neoplasia 19(12): 1022-1032. Thus inhibitors of CDO1 may also be used to inhibit iron-dependent cellular disassembly (e.g., ferroptosis).

In one embodiment, the agent that inhibits iron-dependent cellular disassembly is an inhibitor of lipoxygenase, e.g. arachidonate lipoxygenase (ALOX). In one embodiment, the inhibitor of lipoxygenase is selected from the group consisting of CDC, baicalein, PD-146176, AA-861, and zileuton. In one embodiment, the inhibitor of ALOX is zileuton.

Dipeptidyl-dipeptidase-4 (DPP4) promotes lipid peroxidation through nitric oxide. Accordingly an inhibitor of DPP4 may be used to inhibit iron-dependent cellular disassembly through inhibition of lipid peroxidation. In one embodiment, the inhibitor of DPP4 is selected from vildagliptin, alogliptin, and linagliptin.

In one embodiment, the agent that inhibits iron-dependent cellular disassembly is an inhibitor of glutaminolysis. Glutaminolysis is a series of biochemical reactions by which the amino acid glutamine is lysed to glutamate, aspartate, CO₂, pyruvate, lactate, alanine and citrate. Glutamine is naturally present at high concentrations in human tissues and plasma, and its degradation (via glutaminolysis) provides fuel for the tricarboxylic acid (TCA) cycle and building blocks for essential biosynthetic processes, such as lipid biosynthesis. In the absence of glutamine, or when glutaminolysis is inhibited, cystine starvation and blockage of cystine import fail to induce the accumulation of ROS, lipid peroxidation, and ferroptosis. See Stockwell et al., 2017, Cell 171: 273-285, which is incorporated by reference herein in its entirety.

In one embodiment, the agent that inhibits iron-dependent cellular disassembly is selected from an inhibitor of lipid peroxidation, an inhibitor of glutaminolysis, an inhibitor of lipoxygenase, an inhibitor of cysteine dioxygenase 1 (CDO1), and an inhibitor of DPP4.

In one embodiment, the agent that inhibits iron-dependent cellular disassembly is selected from cycloheximide, beta-mercaptoethanol, dopamine, and selenium.

In one embodiment, the agent that inhibits iron-dependent cellular disassembly has one or more of the following characteristics:

(a) inhibits iron-dependent cellular disassembly of a target cell in vitro and subsequent activation of an immune response in a co-cultured cell;

(b) inhibits iron-dependent cellular disassembly of a target cell in vitro and subsequent activation of co-cultured macrophages, e.g., RAW264.7 macrophages;

(c) inhibits iron-dependent cellular disassembly of a target cell in vitro and subsequent activation of co-cultured monocytes, e.g., THP-1 monocytes;

(d) inhibits iron-dependent cellular disassembly of a target cell in vitro and subsequent activation of co-cultured bone marrow-derived dendritic cells (BMDCs);

(e) inhibits iron-dependent cellular disassembly of a target cell in vitro and subsequent increase in levels or activity of NFkB, IRF and/or STING in a co-cultured cell;

(f) inhibits iron-dependent cellular disassembly of a target cell in vitro and subsequent increase in levels or activity of a pro-immune cytokine in a co-cultured cell; and

(g) inhibits iron-dependent cellular disassembly of a target cell in vitro and subsequent activation of co-cultured CD4+ cells, CD8+ cells and/or CD3+ cells.

### III. Methods of Decreasing Immune Activity

The agents that inhibit iron-dependent cellular disassembly (e.g., ferroptosis) described herein may be used to decrease immune activity in a cell, tissue or in a subject, for example, a subject who would benefit from decreased immune activity. For example, in some aspects, the disclosure relates to a method of decreasing immune activity in a cell, tissue or subject, the method comprising administering to the cell, tissue or subject an agent that inhibits iron-dependent cellular disassembly in an amount sufficient to decrease the immune activity relative to a cell, tissue or subject that is not treated with the agent that inhibits iron-dependent cellular disassembly.

In one embodiment, the subject is in need of decreased immune activity.

In some aspects, the disclosure relates to a method of decreasing immune activity in an immune cell, comprising: contacting a target cell with an agent that inhibits iron-dependent cellular disassembly in an amount sufficient to decrease the immune activity in an immune cell that is exposed to the target cell or to postcellular signaling factors produced by the target cell, wherein the immune activity in the immune cell is decreased relative to an immune cell exposed to a target cell that is undergoing iron-dependent cellular disassembly and is not treated with the agent. In some embodiments, the target cell that is contacted with the agent is undergoing iron-dependent cellular disassembly, or is a target cell in which iron-dependent cellular disassembly has been induced.

In some embodiments, the step of contacting the target cell with the agent is carried out in vitro, ex vivo, or in vivo. In some embodiments, the entire method is carried out in vitro, ex vivo, or in vivo.

Administration of the agent that inhibits iron-dependent cellular disassembly may decrease immune activity by inhibiting production of immunostimulatory postcellular signaling factors by cells undergoing iron-dependent cellular disassembly. These postcellular signaling factors produced by cells undergoing iron-dependent cellular disassembly may increase immune activity through interaction with a broad range of immune cells, including mast cells, Natural Killer (NK) cells, basophils, neutrophils, monocytes, macrophages, dendritic cells, eosinophils, and lymphocytes (e.g. B-lymphocytes (B-cells)), and T-lymphocytes (T-cells)).

Mast cells are a type of granulocyte containing granules rich in histamine and heparin, an anti-coagulant. When activated, a mast cell releases inflammatory compounds from the granules into the local microenvironment. Mast cells play a role in allergy, anaphylaxis, wound healing, angiogenesis, immune tolerance, defense against pathogens, and blood-brain barrier function.

Natural Killer (NK) cells are cytotoxic lymphocytes that lyse certain tumor and vims infected cells without any prior stimulation or immunization. NK cells are also potent producers of various cytokines, e.g. IFN-gamma (IFNγ), TNF-alpha (TNFα), GM-CSF and IL-3. Therefore, NK cells are also believed to function as regulatory cells in the immune system, influencing other cells and responses. In humans, NK cells are broadly defined as CD56+CD3− lymphocytes. The cytotoxic activity of NK cells is tightly controlled by a balance between the activating and inhibitory signals from receptors on the cell surface. A main group of receptors that inhibits NK cell activation are the inhibitory killer immunoglobulin-like receptors (KIRs). Upon recognition of self MHC class I molecules on the target cells, these receptors deliver an inhibitory signal that stops the activating signaling cascade, keeping cells with normal MHC class I expression from NK cell lysis. Activating receptors include the natural cytotoxicity receptors (NCR) and NKG2D that push the balance towards cytolytic action through engagement with different ligands on the target cell surface. Thus, NK cell recognition of target cells is tightly regulated by processes involving the integration of signals delivered from multiple activating and inhibitory receptors.

Monocytes are bone marrow-derived mononuclear phagocyte cells that circulate in the blood for few hours/days before being recruited into tissues. See Wacleche et al., 2018, Viruses (10)2: 65. The expression of various chemokine receptors and cell adhesion molecules at their surface allows them to exit the bone marrow into the blood and to be subsequently recruited from the blood into tissues. Monocytes belong to the innate arm of the immune system providing responses against viral, bacterial, fungal or parasitic infections. Their functions include the killing of pathogens via phagocytosis, the production of reactive oxygen species (ROS), nitric oxide (NO), myeloperoxidase and inflammatory cytokines. Under specific conditions, monocytes can stimulate or inhibit T-cell responses during cancer as well as infectious and autoimmune diseases. They are also involved in tissue repair and neovascularization.

Macrophages engulf and digest substances such as cellular debris, foreign substances, microbes and cancer cells in a process called phagocytosis. Besides phagocytosis, macrophages play a critical role in nonspecific defense (innate immunity) and also help initiate specific defense mechanisms (adaptive immunity) by recruiting other immune cells such as lymphocytes. For example, macrophages are important as antigen presenters to T cells. Beyond increasing inflammation and stimulating the immune system, macrophages also play an important anti-inflammatory role and can decrease immune reactions through the release of cytokines. Macrophages that encourage inflammation are called M1 macrophages, whereas those that decrease inflammation and encourage tissue repair are called M2 macrophages.

Dendritic cells (DCs) play a critical role in stimulating immune responses against pathogens and maintaining immune homeostasis to harmless antigens. DCs represent a heterogeneous group of specialized antigen-sensing and antigen-presenting cells (APCs) that are essential for the induction and regulation of immune responses. In the peripheral blood, human DCs are characterized as cells lacking the T-cell (CD3, CD4, CD8), the B-cell (CD19, CD20) and the monocyte markers (CD14, CD16) but highly expressing HLA-DR and other DC lineage markers (e.g., CD1a, CD1c). See Murphy et al., Janeway's Immunobiology. 8th ed. Garland Science; New York, N.Y., USA: 2012. 868p.

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens through recombination of their genetic material (e.g. to create a T cell receptor and a B cell receptor). This commitment, which exists before the first contact of the immune system with a given antigen, is expressed by the presence of receptors specific for determinants (epitopes) on the antigen on the lymphocyte's surface membrane. Each lymphocyte possesses a unique population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

Lymphocytes include B-lymphocytes (B-cells), which are precursors of antibody-secreting cells, and T-lymphocytes (T-cells).

### B-Lymphocytes (B-Cells)

B-lymphocytes are derived from hematopoietic cells of the bone marrow. A mature B-cell can be activated with an antigen that expresses epitopes that are recognized by its cell surface. The activation process may be direct, dependent on cross-linkage of membrane Ig molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses (Paul, W. E., “Chapter 1: The immune system: an introduction,” Fundamental Immunology, 4th Edition, Ed. Paul, W. E., Lippicott-Raven Publishers, Philadelphia, (1999)).

Cross-linkage dependent B-cell activation requires that the antigen express multiple copies of the epitope complementary to the binding site of the cell surface receptors, because each B-cell expresses Ig molecules with identical variable regions. Such a requirement is fulfilled by other antigens with repetitive epitopes, such as capsular polysaccharides of microorganisms or viral envelope proteins. Cross-linkage-dependent B-cell activation is a major protective immune response mounted against these microbes (Paul, W. E., “Chapter 1: The immune system: an introduction”, Fundamental Immunology, 4th Edition, Ed. Paul, W. E., Lippicott-Raven Publishers, Philadelphia, (1999)).

Cognate help allows B-cells to mount responses against antigens that cannot cross-link receptors and, at the same time, provides costimulatory signals that rescue B cells from inactivation when they are stimulated by weak cross-linkage events. Cognate help is dependent on the binding of antigen by the B-cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T-cells designated as CD4⁺ T-cells. The CD4⁺ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. In addition, T helper cells secrete several cytokines that regulate the growth and differentiation of the stimulated B-cell by binding to cytokine receptors on the B cell (Paul, W. E., “Chapter 1: The immune system: an introduction, “Fundamental Immunology, 4th Edition, Ed. Paul, W. E., Lippicott-Raven Publishers, Philadelphia, (1999)).

During cognate help for antibody production, the CD40 ligand is transiently expressed on activated CD4⁺ T helper cells, and it binds to CD40 on the antigen-specific B cells, thereby transducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in pathogenic autoantibody production in human SLE patients (Desai-Mehta, A. et al., “Hyperexpression of CD40 ligand by B and T cells in human lupus and its role in pathogenic autoantibody production,” J. Clin. Invest. Vol. 97(9), 2063-2073, (1996)).

### T-Lymphocytes (T-Cells)

T-lymphocytes derived from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on T cell expression of specific cell surface molecules and the secretion of cytokines (Paul, W. E., “Chapter 1: The immune system: an introduction”, Fundamental Immunology, 4th Edition, Ed. Paul, W. E., Lippicott-Raven Publishers, Philadelphia, (1999)).

T cells differ from B cells in their mechanism of antigen recognition. Immunoglobulin, the B cell's receptor, binds to individual epitopes on soluble molecules or on particulate surfaces. B-cell receptors see epitopes expressed on the surface of native molecules. While antibody and B-cell receptors evolved to bind to and to protect against microorganisms in extracellular fluids, T cells recognize antigens on the surface of other cells and mediate their functions by interacting with, and altering, the behavior of these antigen-presenting cells (APCs). There are three main types of APCs in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an APC that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the APC for long enough to become activated (“Chapter 24: The adaptive immune system,” Molecular Biology of the Cell, Alberts, B. et al., Garland Science, NY, (2002)).

T-cells are subdivided into two distinct classes based on the cell surface receptors they express. The majority of T cells express T cell receptors (TCR) consisting of α and β-chains. A small group of T cells express receptors made of γ and δ chains. Among the α/β T cells are two sub-lineages: those that express the coreceptor molecule CD4 (CD4⁺ T cells); and those that express CD8 (CD8⁺ T cells). These cells differ in how they recognize antigen and in their effector and regulatory functions.

CD4⁺ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms.

In addition, T cells, particularly CD8⁺ T cells, can develop into cytotoxic T-lymphocytes (CTLs) capable of efficiently lysing target cells that express antigens recognized by the CTLs (Paul, W. E., “Chapter 1: The immune system: an introduction,” Fundamental Immunology, 4th Edition, Ed. Paul, W. E., Lippicott-Raven Publishers, Philadelphia, (1999)).

T cell receptors (TCRs) recognize a complex consisting of a peptide derived by proteolysis of the antigen bound to a specialized groove of a class II or class IMHC protein. CD4⁺ T cells recognize only peptide/class II complexes while CD8⁺ T cells recognize peptide/class I complexes (Paul, W. E., “Chapter 1: The immune system: an introduction,” Fundamental Immunology, 4th Edition, Ed. Paul, W. E., Lippicott-Raven Publishers, Philadelphia, (1999)).

The TCR's ligand (i.e., the peptide/MHC protein complex) is created within APCs. In general, class II MHC molecules bind peptides derived from proteins that have been taken up by the APC through an endocytic process. These peptide-loaded class II molecules are then expressed on the surface of the cell, where they are available to be bound by CD4⁺ T cells with TCRs capable of recognizing the expressed cell surface complex. Thus, CD4⁺ T cells are specialized to react with antigens derived from extracellular sources (Paul, W. E., “Chapter 1: The immune system: an introduction,” Fundamental Immunology, 4th Edition, Ed. Paul, W. E., Lippicott-Raven Publishers, Philadelphia, (1999)).

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally composed of nine amino acids in length, are bound into the class I MHC molecules and are brought to the cell surface, where they can be recognized by CD8⁺ T cells expressing appropriate receptors. This gives the T cell system, particularly CD8⁺ T cells, the ability to detect cells expressing proteins that are different from, or produced in much larger amounts than, those of cells of the remainder of the organism (e.g., viral antigens) or mutant antigens (such as active oncogene products), even if these proteins in their intact form are neither expressed on the cell surface nor secreted (Paul, W. E., “Chapter 1: The immune system: an introduction,” Fundamental Immunology, 4th Edition, Ed. Paul, W. E., Lippicott-Raven Publishers, Philadelphia, (1999)).

T cells can also be classified based on their function as helper T cells; T cells involved in inducing cellular immunity; suppressor T cells; and cytotoxic T cells.

### Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes, and one or more of the generated peptides are loaded into class II MHC molecules, which traffic through this vesicular compartment. The resulting peptide/class II MHC complex is then exported to the B-cell surface membrane. T cells with receptors specific for the peptide/class II molecular complex recognize this complex on the B-cell surface. (Paul, W. E., “Chapter 1: The immune system: an introduction,” Fundamental Immunology, 4th Edition, Ed. Paul, W. E., Lippicott-Raven Publishers, Philadelphia (1999)).

B-cell activation depends both on the binding of the T cell through its TCR and on the interaction of the T-cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B-cell activation. The subsequent events in the B-cell response, including proliferation, Ig secretion, and class switching of the Ig class being expressed, either depend or are enhanced by the actions of T cell-derived cytokines (Paul, W. E., “Chapter 1: The immune system: an introduction,” Fundamental Immunology, 4th Edition, Ed. Paul, W. E., Lippicott-Raven Publishers, Philadelphia, (1999)).

CD4⁺ T cells tend to differentiate into cells that principally secrete the cytokines IL-4, IL-5, IL-6, and IL-10 (T_{H}2 cells) or into cells that mainly produce IL-2, IFN-γ, and lymphotoxin (T_{H}1 cells). The T_{H}2 cells are very effective in helping B-cells develop into antibody-producing cells, whereas the T_{H}1 cells are effective inducers of cellular immune responses, involving enhancement of microbicidal activity of monocytes and macrophages, and consequent increased efficiency in lysing microorganisms in intracellular vesicular compartments. Although CD4⁺ T cells with the phenotype of T_{H}2 cells (i.e., IL-4, IL-5, IL-6 and IL-10) are efficient helper cells, T_{H}1 cells also have the capacity to be helpers (Paul, W. E., “Chapter 1: The immune system: an introduction, “Fundamental Immunology, 4th Edition, Ed. Paul, W. E., Lippicott-Raven Publishers, Philadelphia, (1999)).

### T Cell Involvement in Cellular Immunity Induction

T cells also may act to enhance the capacity of monocytes and macrophages to destroy intracellular microorganisms. In particular, interferon-gamma (IFN-γ) produced by helper T cells enhances several mechanisms through which mononuclear phagocytes destroy intracellular bacteria and parasitism including the generation of nitric oxide and induction of tumor necrosis factor (TNF) production. T_{H1} cells are effective in enhancing the microbicidal action, because they produce IFN-γ. In contrast, two of the major cytokines produced by T_{H2} cells, IL-4 and IL-10, block these activities (Paul, W. E., “Chapter 1: The immune system: an introduction,” Fundamental Immunology, 4th Edition, Ed. Paul, W. E., Lippicott-Raven Publishers, Philadelphia, (1999)).

### Regulatory T (Treg) Cells

Immune homeostasis is maintained by a controlled balance between initiation and downregulation of the immune response. The mechanisms of both apoptosis and T cell anergy (a tolerance mechanism in which the T cells are intrinsically functionally inactivated following an antigen encounter (Schwartz, R. H., “T cell anergy”, Annu. Rev. Immunol., Vol. 21: 305-334 (2003)) contribute to the downregulation of the immune response. A third mechanism is provided by active suppression of activated T cells by suppressor or regulatory CD4⁺ T (Treg) cells (Reviewed in Kronenberg, M. et al., “Regulation of immunity by self-reactive T cells”, Nature, Vol. 435: 598-604 (2005)). CD4⁺ Tregs that constitutively express the IL-2 receptor alpha (IL-2Rα) chain (CD4⁺ CD25⁺) are a naturally occurring T cell subset that are anergic and suppressive (Taams, L. S. et al., “Human anergic/suppressive CD4⁺CD25⁺ T cells: a highly differentiated and apoptosis-prone population”, Eur. J. Immunol. Vol. 31: 1122-1131 (2001)). Depletion of CD4⁺CD25⁺ Tregs results in systemic autoimmune disease in mice. Furthermore, transfer of these Tregs prevents development of autoimmune disease. Human CD4⁺CD25⁺ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4⁺CD25⁺ T cells can be split into suppressive (CD25^{high}) and nonsuppressive (CD25^{low}) cells, according to the level of CD25 expression. A member of the forkhead family of transcription factors, FOXP3, has been shown to be expressed in murine and human CD4⁺CD25⁺ Tregs and appears to be a master gene controlling CD4⁺CD25⁺ Treg development (Battaglia, M. et al., “Rapamycin promotes expansion of functional CD4⁺CD25⁺Foxp3⁺ regulator T cells of both healthy subjects and type 1 diabetic patients”, J. Immunol., Vol. 177: 8338-8347, (2006)).

### Cytotoxic T Lymphocytes

CD8⁺ T cells that recognize peptides from proteins produced within the target cell have cytotoxic properties in that they lead to lysis of the target cells. The mechanism of CTL-induced lysis involves the production by the CTL of perforin, a molecule that can insert into the membrane of target cells and promote the lysis of that cell. Perforin-mediated lysis is enhanced by granzymes, a series of enzymes produced by activated CTLs. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

### Lymphocyte Activation

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the APC.

### T-Memory Cells

Following the recognition and eradication of pathogens through adaptive immune responses, the vast majority (90-95%) of T cells undergo apoptosis with the remaining cells forming a pool of memory T cells, designated central memory T cells (TCM), effector memory T cells (TEM), and resident memory T cells (TRM) (Clark, R. A., “Resident memory T cells in human health and disease”, Sci. Transl. Med., 7, 269rvl, (2015)).

Compared to standard T cells, these memory T cells are long-lived with distinct phenotypes such as expression of specific surface markers, rapid production of different cytokine profiles, capability of direct effector cell function, and unique homing distribution patterns. Memory T cells exhibit quick reactions upon re-exposure to their respective antigens in order to eliminate the reinfection of the offender and thereby restore balance of the immune system rapidly. Increasing evidence substantiates that autoimmune memory T cells hinder most attempts to treat or cure autoimmune diseases (Clark, R. A., “Resident memory T cells in human health and disease”, Sci. Transl. Med., Vol. 7, 269rvl, (2015)).

The agent that inhibits iron-dependent cellular disassembly may decrease immune activity in a tissue or subject by preventing production of postcellular signaling factors that increase the level or activity of immune cells described herein, for example, macrophages, monocytes, dendritic cells, and CD4+, CD8+ or CD3+ cells (e.g. CD4+, CD8+ or CD3+ T cells). For example, in one embodiment, the agent that inhibits iron-dependent cellular disassembly is administered in an amount sufficient to decrease in the tissue or subject one or more of: the level or activity of macrophages, the level or activity of monocytes, the level or activity of dendritic cells, the level or activity of T cells, and the level or activity of CD4+, CD8+ or CD3+ cells (e.g. CD4+, CD8+ or CD3+ T cells).

The agent that inhibits iron-dependent cellular disassembly may also decrease immune activity in a cell, tissue or subject by inhibiting production of postcellular signaling factors that increase the level or activity of a pro-immune cytokine. For example, in some embodiments, the agent that inhibits iron-dependent cellular disassembly is administered in an amount sufficient to decrease in a cell, tissue or subject the level or activity of a pro-immune cytokine. In one embodiment, the pro-immune cytokine is selected from IFN-α, IL-1, IL-12, IL-18, IL-2, IL-15, IL-4, IL-6, TNF-α, IL-17 and GMCSF.

The agent that inhibits iron-dependent cellular disassembly may also decrease immune activity in a cell, tissue or subject by inhibiting production of postcellular signaling factors that increase the level or activity of positive regulators of the immune response such as nuclear factor kappa-light-chain-enhancer of activated B cells (NFkB), interferon regulatory factor (IRF), and stimulator of interferon genes (STING). For example, in some embodiments, the agent that inhibits iron-dependent cellular disassembly is administered in an amount sufficient to decrease in a cell, tissue or subject the level or activity of NFkB, IRF and/or STING.

In some aspects, the disclosure relates to a method of decreasing the level or activity of NFkB in a cell, tissue or subject, comprising administering to the cell, tissue or subject an agent that inhibits iron-dependent cellular disassembly in an amount sufficient to decrease the level or activity of NFkB relative to a cell, tissue or subject that is not treated with the agent that inhibits iron-dependent cellular disassembly.

In one embodiment, the subject is in need of a decreased level or activity of NFkB.

In one embodiment, the level or activity of NFkB is decreased by at least 10%, 20%, 30%, 40%, 50%, 60%, 70%, 80%, 90% or 99%, or by at least 2-fold, 4-fold, 6-fold, 8-fold, or 10-fold relative to a cell, tissue or subject that is not treated with the agent that inhibits iron-dependent cellular disassembly.

In some aspects, the disclosure relates to a method of decreasing the level or activity of IRF or STING in a cell, tissue or subject, comprising administering to the cell, tissue or subject an agent that inhibits iron-dependent cellular disassembly in an amount sufficient to decrease the level or activity of IRF or STING relative to a cell, tissue or subject that is not treated with the agent that inhibits iron-dependent cellular disassembly.

In one embodiment, the subject is in need of a decreased level or activity of IRF or STING.

In one embodiment, the level or activity of IRF or STING is increased by at least 10%, 20%, 30%, 40%, 50%, 60%, 70%, 80%, 90% or 99%, or by at least 2-fold, 4-fold, 6-fold, 8-fold, or 10-fold relative to a cell, tissue or subject that is not treated with the agent that inhibits iron-dependent cellular disassembly.

In some aspects, the disclosure relates to a method of decreasing the level or activity of macrophages, monocytes, dendritic cells or T cells in a tissue or subject, comprising administering to the tissue or subject an agent that inhibits iron-dependent cellular disassembly in an amount sufficient to increase the level or activity of macrophages, monocytes, dendritic cells or T cells relative to a tissue or subject that is not treated with the agent that inhibits iron-dependent cellular disassembly.

In one embodiment, the subject is in need of a decreased level or activity of macrophages, monocytes, dendritic cells or T cells.

In one embodiment, the level or activity of macrophages, monocytes, dendritic cells, or T cells is decreased by at least 10%, 20%, 30%, 40%, 50%, 60%, 70%, 80%, 90% or 99%, or by at least 2-fold, 4-fold, 6-fold, 8-fold, or 10-fold relative to a tissue or subject that is not treated with the agent that inhibits iron-dependent cellular disassembly.

In some aspects, the disclosure relates to a method of decreasing the level or activity of CD4+, CD8+, or CD3+ cells in a tissue or subject, comprising administering to the subject an agent that inhibits iron-dependent cellular disassembly in an amount sufficient to decrease the level or activity of CD4+, CD8+, or CD3+ cells relative to a tissue or subject that is not treated with the agent that inhibits iron-dependent cellular disassembly.

In one embodiment, the subject is in need of a decreased level or activity of CD4+, CD8+, or CD3+ cells.

In one embodiment, the level or activity of CD4+, CD8+, or CD3+ cells is decreased by at least 10%, 20%, 30%, 40%, 50%, 60%, 70%, 80%, 90% or 99%, or by at least 2-fold, 4-fold, 6-fold, 8-fold, or 10-fold relative to a tissue or subject that is not treated with the agent that inhibits iron-dependent cellular disassembly.

In some aspects, the disclosure relates to a method of decreasing the level or activity of a pro-immune cytokine in a cell, tissue or subject, comprising administering to the cell, tissue or subject an agent that inhibits iron-dependent cellular disassembly in an amount sufficient to decrease the level or activity of the pro-immune cytokine relative to a cell, tissue or subject that is not treated with the agent that inhibits iron-dependent cellular disassembly.

In one embodiment, the subject is in need of a decreased level or activity of a pro-immune cytokine.

In one embodiment, the level or activity of the pro-immune cytokine is decreased by at least 10%, 20%, 30%, 40%, 50%, 60%, 70%, 80%, 90% or 99%, or by at least 2-fold, 4-fold, 6-fold, 8-fold, or 10-fold relative to a cell, tissue or subject that is not treated with the agent that inhibits iron-dependent cellular disassembly.

In one embodiment, the pro-immune cytokine is selected from IFN-α, IL-1, IL-12, IL-18, IL-2, IL-15, IL-4, IL-6, TNF-α, IL-17 and GMCSF.

In one embodiment, the method further includes, before the administration, evaluating the cell, tissue or subject for one or more of: the level or activity of NFkB; the level or activity of macrophages; the level or activity of monocytes; the level or activity of dendritic cells; the level or activity of CD4+ cells, CD8+ cells, or CD3+ cells; the level or activity of T cells; and the level or activity of a pro-immune cytokine.

In one embodiment, the method further includes, after the administration, evaluating the cell, tissue or subject for one or more of: the level or activity of NFkB; the level or activity of macrophages; the level or activity of monocytes; the level or activity of dendritic cells; the level or activity of CD4+ cells, CD8+ cells or CD3+ cells; the level or activity of T cells; and the level or activity of a pro-immune cytokine.

Methods of measuring the level or activity of NFkB, IRF or STING; the level or activity of macrophages; the level or activity of monocytes; the level or activity of dendritic cells; the level or activity of CD4+ cells, CD8+ cells or CD3+ cells; the level or activity of T cells; and the level or activity of a pro-immune cytokine are known in the art.

For example, the protein level or activity of NFkB, IRF or STING may be measured by suitable techniques known in the art including ELISA, Western blot or in situ hydridization. The level of a nucleic acid (e.g. an mRNA) encoding NFkB, IRF or STING may be measured using suitable techniques known in the art including polymerase chain reaction (PCR) amplification reaction, reverse-transcriptase PCR analysis, quantitative real-time PCR, single-strand conformation polymorphism analysis (SSCP), mismatch cleavage detection, heteroduplex analysis, Northern blot analysis, in situ hybridization, array analysis, deoxyribonucleic acid sequencing, restriction fragment length polymorphism analysis, and combinations or sub-combinations thereof.

Methods for measuring the level and activity of macrophages are described, for example, in Chitu et al., 2011, Curr Protoc Immunol 14: 1-33. The level and activity of monocytes may be measured by flow cytometry, as described, for example, in Henning et al., 2015, Journal of Immunological Methods 423: 78-84. The level and activity of dendritic cells may be measured by flow cytometry, as described, for example in Dixon et al., 2001, Infect Immun. 69(7): 4351-4357. Each of these references is incorporated by reference herein in its entirety.

The level or activity of T cells may be assessed using a human CD4+ T-cell-based proliferative assay. For example, cells are labeled with the fluorescent dye 5,6-carboxyfluorescein diacetate succinimidyl ester (CFSE). Those cells that proliferate show a reduction in CFSE fluorescence intensity, which is measured directly by flow cytometry. Alternatively, radioactive thymidine incorporation can be used to assess the rate of growth of the T cells. Functional activity may also be assessed using an in vitro Treg suppression assay. Such an assay is described in Collinson and Vignali (Methods Mol Biol. 2011; 707: 21-37, incorporated by reference in its entirety herein).

Regulatory T cells (Tregs) are a class of CD4+CD25+ T cells that suppress the activity of other immune cells. Tregs are central to immune system homeostasis, and play a major role in maintaining tolerance to self-antigens and in modulating the immune response to foreign antigens. Multiple autoimmune and inflammatory diseases, including Type 1 Diabetes (T1D), Systemic Lupus Erythematosus (SLE), and Graft-versus-Host Disease (GVHD) have been shown to have a deficiency of Treg cell numbers or Treg function. One assay to determine Treg activity measures the phosphorylation of the signal transduction protein STAT5, measured by flow cytometry with an antibody specific for the phosphorylated protein (pSTAT5). STAT5 is essential for Treg development, and a constitutively activated form of STAT5 expressed in CD4+CD25+ cells is sufficient for the production of Treg cells in the absence of IL-2 (Mahmud, S. A., et al., 2013, JAKSTAT 2:e23154). Therefore, measurement of phosphorylated STAT5 (pSTAT5) in Treg cells provides a method for determining activation of these cells. Another assay for functional activation measures proliferation of Treg cells. Treg proliferation can be measured by tritiated thymidine incorporation into purified Treg cells, by an increase in Treg cell numbers in a mixed population of cells measured by flow cytometry and the frequencies of CD4+CD25+FOXP3+ or the CD4+CD25+CD127− marker phenotypes, by increased expression in Treg cells of proliferation-associated cell cycle proteins, such as Ki-67, or by measurement of the cell division-associated dilution of a vital fluorescent dye such as carboxyfluorescein succinimidyl ester (CFSE) by flow cytometry in Treg cells. Another assay for functional activation of Tregs is the increased stability of Tregs. pTreg cells are thought by some to be unstable, and have the potential to differentiate into Th1 and Th17 effector T cells. Activation of Tregs can stabilize Tregs and prevent this differentiation (Chen, Q., et al., 2011, J Immunol. 186:6329-37). Another outcome of stimulation of Tregs is the stimulation of the level of Treg functional effector molecules, such as CTLA4, GITR, LAG3, TIGIT, IL-10, CD39, and CD73, which contribute to the immunosuppressive activity of Tregs. Production of these effector molecules by Tregs may be determined by methods known in the art, such as ELISA.

The level or activity of a pro-immune cytokine may be quantified, for example, in CD8+ T cells. In embodiments, the pro-immune cytokine is selected from interferon alpha (IFN-α), interleukin-1 (IL-1), IL-12, IL-18, IL-2, IL-15, IL-4, IL-6, tumor necrosis factor alpha (TNF-α), IL-17, and granulocyte-macrophage colony-stimulating factor (GMCSF). Quantitation can be carried out using the ELISPOT (enzyme-linked immunospot) technique, that detects T cells that secrete a given cytokine (e.g. IFN-α) in response to an antigenic stimulation. T cells are cultured with antigen-presenting cells in wells which have been coated with, e.g., anti-IFN-α antibodies. The secreted IFN-α is captured by the coated antibody and then revealed with a second antibody coupled to a chromogenic substrate. Thus, locally secreted cytokine molecules form spots, with each spot corresponding to one IFN-α-secreting cell. The number of spots allows one to determine the frequency of IFN-α-secreting cells specific for a given antigen in the analyzed sample. The ELISPOT assay has also been described for the detection of TNF-α, interleukin-4 (IL-4), IL-6, IL-12, and GMCSF.

### IV. Methods of Treating Disorders

Applicants have shown that treatment of cells with agents that induce iron-dependent cellular disassembly results in the production and release of postcellular signaling factors that increase immune activity. These results suggest that inhibition of iron-dependent cellular disassembly would decrease immune activity. Accordingly, agents that inhibit iron-dependent cellular disassembly and decrease immune activity may be used in the treatment of disorders that may benefit from decreased immune activity, such as inflammatory disorders or conditions including inflammation, acute organ injury, tissue damage, sepsis, ischemia, atherosclerosis, neurodegenerative disorders, and immune-related diseases or conditions.

In certain embodiments, the subject treated by the methods of the present disclosure has a disorder associated with iron-dependent cellular disassembly (e.g. ferroptosis). In some embodiments, the disorder is a disorder in which iron-dependent cellular disassembly (e.g. ferroptosis) is detrimental. Iron-dependent cellular disassembly may be evaluated in a subject by collecting a sample from the subject (e.g. a blood or tissue sample) and analyzing the sample for the presence of iron-dependent cellular disassembly using methods known in the art. For example, because iron-dependent cellular disassembly may result from lethal lipid peroxidation, measuring lipid peroxidation provides one method of identifying subjects having a disorder associated with iron-dependent cellular disassembly. C11-BODIPY and Liperfluo are lipophilic ROS sensors that provide a rapid, indirect means to detect lipid ROS (Dixon et al., 2012, Cell 149: 1060-1072). Liquid chromatography (LC)/tandem mass spectrometry (MS) analysis can also be used to detect specific oxidized lipids directly (Friedmann Angeli et al., 2014, Nat. Cell Biol. 16: 1180-1191; Kagan et al., 2017, Nat. Chem. Biol. 13: 81-90). Isoprostanes and malondialdehyde (MDA) may also be used to measure lipid peroxidation (Milne et al., 2007, Nat. Protoc. 2: 221-226; Wang et al., 2017, Hepatology 66(2): 449-465). Kits for measuring MDA are commercially available (Beyotime, Haimen, China).

Other useful assays for identifying iron-dependent cellular disassembly in a subject include measuring iron abundance and GPX4 activity. Iron abundance can be measured using inductively coupled plasma-MS or calcein AM quenching, as well as other specific iron probes (Hirayama and Nagasawa, 2017, J. Clin. Biochem. Nutr. 60: 39-48; Spangler et al., 2016, Nat. Chem. Biol. 12: 680-685), while GPX4 activity can be detected using phosphatidylcholine hydroperoxide reduction in cell lysates using LC-MS (Yang et al., 2014, Cell 156: 317-331). In addition, iron-dependent cellular disassembly may be evaluated in a subject by measuring glutathione (GSH) content. GSH may be measured, for example, by using the commercially available GSH-Glo Glutathione Assay (Promega, Madison, Wis.).

Iron-dependent cellular disassembly may also be evaluated in a subject by measuring the expression of one or more marker proteins. Suitable marker proteins include, but are not limited to, glutathione peroxidase 4 (GPX4), prostaglandin-endoperoxide synthase 2 (PTGS2), and cyclooxygenase-2 (COX-2). The level of expression of the marker protein or a nucleic acid encoding the marker protein may be determined using suitable techniques known in the art including, but not limited to polymerase chain reaction (PCR) amplification reaction, reverse-transcriptase PCR analysis, quantitative real-time PCR, single-strand conformation polymorphism analysis (SSCP), mismatch cleavage detection, heteroduplex analysis, Northern blot analysis, Western blot analysis, in situ hybridization, array analysis, deoxyribonucleic acid sequencing, restriction fragment length polymorphism analysis, and combinations or sub-combinations thereof.

In some embodiments, evaluating the subject for the presence of a disorder associated with iron-dependent cellular disassembly (e.g. a disorder in which iron-dependent cellular disassembly is detrimental) comprises measuring the level or activity of a marker of iron-dependent cellular disassembly, e.g., a marker selected from the group consisting of lipid peroxidation, reactive oxygen species (ROS), isoprostanes, malondialdehyde (MDA), iron, glutathione peroxidase 4 (GPX4), prostaglandin-endoperoxide synthase 2 (PTGS2), cyclooxygenase-2 (COX-2) and glutathione (GSH), in the sample collected from the subject.

In some embodiments, evaluating the subject for the presence of a disorder associated with iron-dependent cellular disassembly (e.g. a disorder in which iron-dependent cellular disassembly is detrimental) comprises comparing the level or activity of the marker in the sample collected from the subject to the level or activity of the marker in a control sample. A control sample may be, for example, a sample from a healthy subject, or a sample from a subject that does not have a disorder associated with iron-dependent cellular disassembly.

In one embodiment, an increase in the level or activity of a marker selected from the group consisting of lipid peroxidation, isoprostanes, reactive oxygen species (ROS), iron, PTGS2 and COX-2, or a decrease in the level or activity of a marker selected from the group consisting of GPX4, MDA and GSH in the sample from the subject relative to a control sample indicates that the subject has a disorder associated with iron-dependent cellular disassembly.

In one embodiment, evaluating the subject for the presence of a disorder associated with iron-dependent cellular disassembly comprises measuring lipid peroxides in the sample collected from the subject.

In one embodiment, an increase in the level of lipid peroxides in the sample from the subject relative to a control sample indicates that the subject has a disorder associated with iron-dependent cellular disassembly.

In some aspects, the present disclosure relates to a method of treating a disorder or condition associated with iron-dependent cellular disassembly (e.g. a disorder in which iron-dependent cellular disassembly is detrimental) in a subject comprising administering to the subject an agent that inhibits iron-dependent cellular disassembly in an amount sufficient to treat the disorder or condition in the subject.

Methods of treating a disorder or condition associated with iron-dependent cellular disassembly may further comprise evaluating the subject for the presence of a disorder associated with iron-dependent cellular disassembly using any one or more of the methods described above.

Inflammatory Diseases or Conditions

In some aspects, the present disclosure relates to a method of treating an inflammatory disease or condition comprising administering to the subject an agent that inhibits iron-dependent cellular disassembly in an amount sufficient to treat the inflammatory disease or condition in the subject. In some embodiments, the inflammatory disease or condition is an inflammatory disease or condition in which iron-dependent cellular disassembly (e.g. ferroptosis) is detrimental.

In some embodiments, the inflammatory disease or condition is selected from the group consisting of inflammation (e.g. sterile inflammation), acute organ injury, tissue damage, sepsis, ischemia, and atherosclerosis.

In some embodiments, the inflammatory disease is an autoimmune disease or immune-related disease or condition. Autoimmune diseases are diseases in which the immune system attacks its own proteins, cells, and tissues. Autoimmune diseases include diseases that affect organs such as the heart, kidney, liver, lung, reproductive organs, digestive system, or skin. Autoimmune diseases include diseases that affect glands, including the endocrine, adrenal, thyroid, salivary and exocrine glands, and the pancreas. Autoimmune diseases can also be multi-glandular. Autoimmune diseases can target one or more tissues, for example connective tissue, muscle, or blood. Autoimmune diseases can target the nervous system or eyes, ears or vascular system. Autoimmune diseases can also be systemic, affecting multiple organs, tissues and/or systems. In some embodiments, an autoimmune disease or condition is an inflammatory disease or condition.

Non-limiting examples of autoimmune or immune-related diseases or conditions include systemic lupus erythematosus, rheumatoid arthritis, Type I diabetes, Type II diabetes, multiple sclerosis (MS), allergies, asthma, psoriasis, amyotrophic lateral sclerosis (ALS), organ transplant/graft-vs-host disease (GVHD), and ulcerative colitis.

In some embodiments, the immune-related condition is an allergy or allergic condition, for example, an allergy or allergic condition in which iron-dependent cellular disassembly (e.g. ferroptosis) is detrimental. An allergy or allergic condition is a hypersensitive reaction to allergens (e.g. lipids or proteins) in the environment. Allergens are antigens to which atopic patients respond with IgE antibody responses subsequently leading to allergic reactions. Allergens include environmental allergens (e.g. house dust mite, birch pollen, grass pollen, cat antigens, cockroach antigens), or food allergens (e.g. cow milk, peanut, shrimp, soybean), or a combination thereof. IgE molecules are important in allergic responses because of their role in effector cell (mast cell, basophiles and eosinophiles) activation. Allergies and allergic conditions include but are not limited to asthma, chronic obstructive pulmonary disease, hay fever (seasonal rhinitis), hives, and eczema.

In some embodiments, the immune-related condition is an autoinflammatory condition. Autoinflammatory conditions result from a dysfunction in the innate immune system, and constitute a broad range of genetically mediated conditions characterized by recurrent attacks of systemic inflammation with primary physical manifestations of fever, rash, serositis, lymphadenopathy, and musculoskeletal symptoms. Genetic mutations that usually cause some dysregulation of the innate immune system underlie the etiology of autoinflammatory conditions.

In some embodiments, the disorder associated with iron-dependent cellular disassembly is associated with neuroinflammation. Neuroinflammation is a chronic inflammation of the nervous system and is often associated with brain injury and neurodegenerative disorders. Neurodegenerative disorders involve the progressive loss of structure or function of neurons, and may involve death of neurons. In some embodiments, the disorder associated with iron-dependent cellular disassembly is a neurodegenerative disorder, e.g. Parkinson's disease, Huntington's disease, or Alzheimer's disease. See Chen et al., 2015, J. Biol. Chem. 290: 28097-28106, which is incorporated by reference herein in its entirety. In some embodiments, the condition associated with iron-dependent cellular disassembly is traumatic or hemorrhagic brain injury. See Stockwell et al., 2017, Cell 171: 273-285, which is incorporated by reference herein in its entirety. In some embodiments, the disorder associated with iron-dependent cellular disassembly is not one or more of a neurodegenerative disorder (e.g. Parkinson's disease, Huntington's disease, or Alzheimer's disease), ischemia, and traumatic or hemorrhagic brain injury.

### V. Pharmaceutical Compositions and Modes of Administration

The pharmaceutical compositions described herein may be administered to a subject in any suitable formulation. These include, for example, liquid, semi-solid, and solid dosage forms, The preferred form depends on the intended mode of administration and therapeutic application.

In certain embodiments the composition is suitable for oral administration. In certain embodiments, the formulation is suitable for parenteral administration, including topical administration and intravenous, intraperitoneal, intramuscular, and subcutaneous, injections. In a particular embodiment, the composition is suitable for intravenous administration.

Pharmaceutical compositions for parenteral administration include aqueous solutions of the active compounds in water-soluble form. For intravenous administration, the formulation may be an aqueous solution. The aqueous solution may include Hank's solution, Ringer's solution, phosphate buffered saline (PBS), physiological saline buffer or other suitable salts or combinations to achieve the appropriate pH and osmolarity for parenterally delivered formulations. Aqueous solutions can be used to dilute the formulations for administration to the desired concentration. The aqueous solution may contain substances which increase the viscosity of the solution, such as sodium carboxymethyl cellulose, sorbitol, or dextran. In some embodiments, the formulation includes a phosphate buffer saline solution which contains sodium phosphate dibasic, potassium phosphate monobasic, potassium chloride, sodium chloride and water for injection.

Formulations suitable for topical administration include liquid or semi-liquid preparations suitable for penetration through the skin, such as liniments, lotions, creams, ointments or pastes, and drops suitable for administration to the eye, ear, or nose. Formulations suitable for oral administration include preparations containing an inert diluent or an assimilable edible carrier. The formulation for oral administration may be enclosed in hard or soft shell gelatin capsule, or it may be compressed into tablets, or it may be incorporated directly with the food of the diet. When the dosage unit form is a capsule, it may contain, in addition to materials of the above type, a liquid carrier. Various other materials may be present as coatings or to otherwise modify the physical form of the dosage unit. Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers including excipients and auxiliaries which facilitate processing of the active compounds into preparations which can be used pharmaceutically.

As will be readily apparent to one skilled in the art, the useful in vivo dosage to be administered and the particular mode of administration will vary depending upon the age, body weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials, animal models, and in vitro studies.

In certain embodiments, the composition is delivered orally. In certain embodiments, the composition is administered parenterally. In certain embodiments, the compositions is delivered by injection or infusion. In certain embodiments, the composition is delivered topically including transmucosally. In certain embodiments, the composition is delivered by inhalation. In one embodiment, the compositions provided herein may be administered by injecting directly to a tumor. In some embodiments, the compositions may be administered by intravenous injection or intravenous infusion. In certain embodiments, administration is systemic. In certain embodiments, administration is local.

### VI. Methods for Identification of Immunoinhibitory Agents that Inhibit Iron-Dependent Cellular Disassembly

In addition to the agents that inhibit iron-dependent cellular disassembly known in the art and described herein, the disclosure further relates to methods for identifying other compounds that inhibit iron-dependent cellular disassembly and decrease immune activity.

For example, in certain aspects, the disclosure relates to a method of screening for an immunoinhibitory agent, the method comprising:

(a) providing a plurality of test agents (e.g., a library of test agents);
(b) evaluating each of the plurality of test agents for the ability to inhibit iron-dependent cellular disassembly (e.g. ferroptosis);
(c) selecting as a candidate immunoinhibitor agent a test agent that decreases iron-dependent cellular disassembly (e.g. ferroptosis); and
(d) evaluating the candidate immunostimulatory agent for the ability to decrease an immune response.

In some embodiments, evaluating the test agents for the ability to inhibit iron-dependent cellular disassembly (e.g. ferroptosis) comprises contacting cells or tissue with each of the plurality of test agents.

In some embodiments, evaluating the test agents for the ability to inhibit iron-dependent cellular disassembly (e.g. ferroptosis) comprises administering each of the plurality of test agents to a subject, for example, an animal.

The test agents may be evaluated in a cell, tissue or subject by measuring the ability of a test agent to inhibit induction of iron-dependent cellular disassembly by another compound, i.e. an agent that induces iron-dependent cellular disassembly. For example, in some embodiments, evaluating the test agents for the ability to inhibit iron-dependent cellular disassembly further comprises contacting cells or tissue with an agent that induces iron-dependent cellular disassembly, or administering to a subject an agent that induces iron-dependent cellular assembly. The cells, tissue or subject may then be evaluated for the ability of the test agent to block induction of iron-dependent cellular disassembly by the agent that induces iron-dependent cellular disassembly. The agent that induces iron-dependent cellular disassembly may be administered to the cell, tissue or subject before, after, or concurrently with the test agent.

Several methods are known in the art and may be employed for identifying cells undergoing iron-dependent cellular disassembly (e.g., ferroptosis) and distinguishing from other types of cellular disassembly and/or cell death through detection of particular markers. (See, for example, Stockwell et al., 2017, Cell 171: 273-285, incorporated by reference herein in its entirety). For example, because iron-dependent cellular disassembly may result from lethal lipid peroxidation, measuring lipid peroxidation provides one method of identifying cells undergoing iron-dependent cellular disassembly. C11-BODIPY and Liperfluo are lipophilic ROS sensors that provide a rapid, indirect means to detect lipid ROS (Dixon et al., 2012, Cell 149: 1060-1072). Liquid chromatography (LC)/tandem mass spectrometry (MS) analysis can also be used to detect specific oxidized lipids directly (Friedmann Angeli et al., 2014, Nat. Cell Biol. 16: 1180-1191; Kagan et al., 2017, Nat. Chem. Biol. 13: 81-90). Isoprostanes and malondialdehyde (MDA) may also be used to measure lipid peroxidation (Milne et al., 2007, Nat. Protoc. 2: 221-226; Wang et al., 2017, Hepatology 66(2): 449-465). Kits for measuring MDA are commercially available (Beyotime, Haimen, China).

Other useful assays for studying iron-dependent cellular disassembly include measuring iron abundance and GPX4 activity. Iron abundance can be measured using inductively coupled plasma-MS or calcein AM quenching, as well as other specific iron probes (Hirayama and Nagasawa, 2017, J. Clin. Biochem. Nutr. 60: 39-48; Spangler et al., 2016, Nat. Chem. Biol. 12: 680-685), while GPX4 activity can be detected using phosphatidylcholine hydroperoxide reduction in cell lysates using LC-MS (Yang et al., 2014, Cell 156: 317-331). In addition, iron-dependent cellular disassembly may be evaluated by measuring glutathione (GSH) content. GSH may be measured, for example, by using the commercially available GSH-Glo Glutathione Assay (Promega, Madison, Wis.).

Iron-dependent cellular disassembly may also be evaluated by measuring the expression of one or more marker proteins. Suitable marker proteins include, but are not limited to, glutathione peroxidase 4 (GPX4), prostaglandin-endoperoxide synthase 2 (PTGS2), and cyclooxygenase-2 (COX-2). The level of expression of the marker protein or a nucleic acid encoding the marker protein may be determined using suitable techniques known in the art including, but not limited to polymerase chain reaction (PCR) amplification reaction, reverse-transcriptase PCR analysis, quantitative real-time PCR, single-strand conformation polymorphism analysis (SSCP), mismatch cleavage detection, heteroduplex analysis, Northern blot analysis, Western blot analysis, in situ hybridization, array analysis, deoxyribonucleic acid sequencing, restriction fragment length polymorphism analysis, and combinations or sub-combinations thereof.

In some embodiments, evaluating the test agents for the ability to inhibit iron-dependent cellular disassembly comprises measuring the level or activity of a marker of iron-dependent cellular disassembly, e.g., a marker selected from the group consisting of lipid peroxidation, reactive oxygen species (ROS), isoprostanes, malondialdehyde (MDA), iron, glutathione peroxidase 4 (GPX4), prostaglandin-endoperoxide synthase 2 (PTGS2), cyclooxygenase-2 (COX-2) and glutathione (GSH), in the cells or tissue contacted with the test agent.

In some embodiments, evaluating the test agents for the ability to inhibit iron-dependent cellular disassembly comprises comparing the level or activity of the marker in the cells or tissue contacted with the test agent to the level or activity of the marker in a control cell or tissue that has not been contacted with the test agent.

In one embodiment, a decrease in the level or activity of a marker selected from the group consisting of lipid peroxidation, isoprostanes, reactive oxygen species (ROS), iron, PTGS2 and COX-2, or an increase in the level or activity of a marker selected from the group consisting of GPX4, MDA and GSH indicates that the test agent is an agent that inhibits iron-dependent cellular disassembly.

In one embodiment, evaluating the test agents for the ability to inhibit iron-dependent cellular disassembly comprises measuring lipid peroxides in the cells or tissue contacted with the test agent.

In one embodiment, a decrease in the level of lipid peroxides in the cells or tissue contacted with the test agent indicates that the test agent is an agent that inhibits iron-dependent cellular disassembly.

In one embodiment, evaluating the candidate immunoinhibitory agent for the ability to decrease an immune response comprises evaluating the test agent that inhibits iron-dependent cellular disassembly for immunoinhibitory activity. Any of the methods described herein for evaluating immune response may be used to evaluate the immunoinhibitory activity of the test agent.

For example, in one embodiment, evaluating the candidate immunoinhibitory agent comprises culturing an immune cell together with cells contacted with the selected candidate immunoinhibitory agent or exposing an immune cell to postcellular signaling factors produced by cells contacted with the selected candidate immunoinhibitory agent and measuring the level or activity of NFκB, IRF or STING in the immune cell.

In one embodiment, the immune cell is a THP-1 cell. For example, NFκB and IRF activity may be measured in commercially available THP1-Dual cells (InvivoGen, San Diego, Calif.). THP1-Dual cells are human monocyte cells that induce reporter proteins upon activation of either NFKB or IRF pathways. The THP-1 cells may be cultured with cells contacted with the selected candidate immunoinhibitory agent or exposed to postcellular signaling factors produced by cells contacted with the selected candidate immunoinhibitory agent and then mixed with either 200 μl QuantiBlue (InvivoGen, San Diego, Calif.) or 50 μl QuantiLuc for detection of NFKB and IRF activity. NFKB and IRF activity may be quantified by measuring absorbance or luminescence on a Molecular Devices plate reader.

In one embodiment, evaluating the candidate immunoinhibitory agent comprises culturing T cells together with cells contacted with the selected candidate immunoinhibitory agent or exposing T cells to postcellular signaling factors produced by cells contacted with the selected candidate immunoinhibitory agent and measuring the activation and proliferation of the T cells.

In one embodiment, the immune cell is a macrophage. For example, NFκB and IRF activity may be measured in commercially available Raw-Dual™ and J774-Dual™ macrophage cells (InvivoGen, San Diego, Calif.). Raw-Dual™ and J774-Dual™ cells are mouse macrophage cell lines that induce reporter proteins upon activation of either NFKB or IRF pathways. The macrophage cells may be cultured with cells contacted with the selected candidate immunoinhibitory agent or exposed to postcellular signaling factors produced by cells contacted with the selected candidate immunoinhibitory agent and then mixed with either 200 μl QuantiBlue (InvivoGen, San Diego, Calif.) or 50 μl QuantiLuc for detection of NFKB and IRF activity. NFKB and IRF activity may be quantified by measuring absorbance or luminescence on a Molecular Devices plate reader.

In one embodiment, the immune cell is a Dendritic Cell. For example, costimulatory markers (e.g. CD80, CD86) or markers of enhanced antigen presentation (e.g. MHCII) can be measured in dendritic cells by flow cytometry. The dendritic cells may be cultured with cells contacted with the selected candidate immunoinhibitory agent or exposed to compounds produced by cells contacted with the selected candidate immunoinhibitory agent and then stained with antibodies specific to cell surface markers indicative of activation status. Subsequently, the expression level of these markers is determined by flow cytometry.

Candidate immunoinhibitory agents may also be evaluated by measuring pro-immune cytokine levels in macrophages and/or dendritic cells. For example, in some embodiments, evaluating candidate immunoinhibitory agents comprises culturing macrophage cells and/or dendritic cells with cells contacted with the selected candidate immunoinhibitory agent or contacting macrophage cells and/or dendritic cells with postcellular signaling factors produced by cells contacted with the selected candidate immunoinhibitory agent and measuring levels of pro-immune cytokines (e.g. IFN-α, IL-1, IL-12, IL-18, IL-2, IL-15, IL-4, IL-6, TNF-α, IL-17 and GMCSF). Pro-immune cytokine levels may be determined by methods known in the art, such as ELISA.

Agents that Induce Iron-Dependent Cellular Disassembly

A broad range of agents that induce iron-dependent cellular disassembly, e.g., ferroptosis, are known in the art, and are useful in the methods for identifying immunoinhibitory agents that inhibit iron-dependent cellular disassembly described herein. For example, two oncogenic RAS Selective Lethal (RSL) small molecules named eradicator of Ras and ST (erastin) and Ras Selective Lethal 3 (RSL3) were initially identified as small molecules that are selectively lethal to cells expressing oncogenic mutant RAS proteins, a family of small GTPases that are commonly mutated in cancer. (See Cao et al., 2016, Cell Mol Life Sci 73: 2195-2209, incorporated in its entirety herein.) Specifically, in engineered human fibroblast cell lines, the small molecule erastin was found to induce preferential lethality in cells overexpressing oncogenic HRAS (see Dolma et al., 2003, Cancer Cell. 3:285-296, incorporated in its entirety herein). Erastin functionally inhibits the cystine-glutamate antiporter system Xc−. System Xc− is a heterodimeric cell surface amino acid antiporter composed of the twelve-pass transmembrane transporter protein SLC7A11 (xCT) linked by a disulfide bridge to the single-pass transmembrane regulatory protein SLC3A2 (4F2hc, CD98hc). Antiporter system Xc-imports extracellular cystine, the oxidized form of cysteine, in exchange for intracellular glutamate. (See Cao et al., 2016, Cell Mol Life Sci 73: 2195-2209, incorporated in its entirety herein.) Cells treated with erastin are deprived of cysteine and are unable to synthesize the antioxidant glutathione. Depletion of glutathione eventually leads to excessive lipid peroxidation and increased ROS which triggers iron-dependent cellular disassembly. Erastin-induced ferroptotic cell death is distinct from apoptosis, necrosis, and autophagy, based on morphological, biochemical, and genetic criteria. (See Yang et al., 2014, Cell 156: 317-331, incorporated in its entirety herein.)

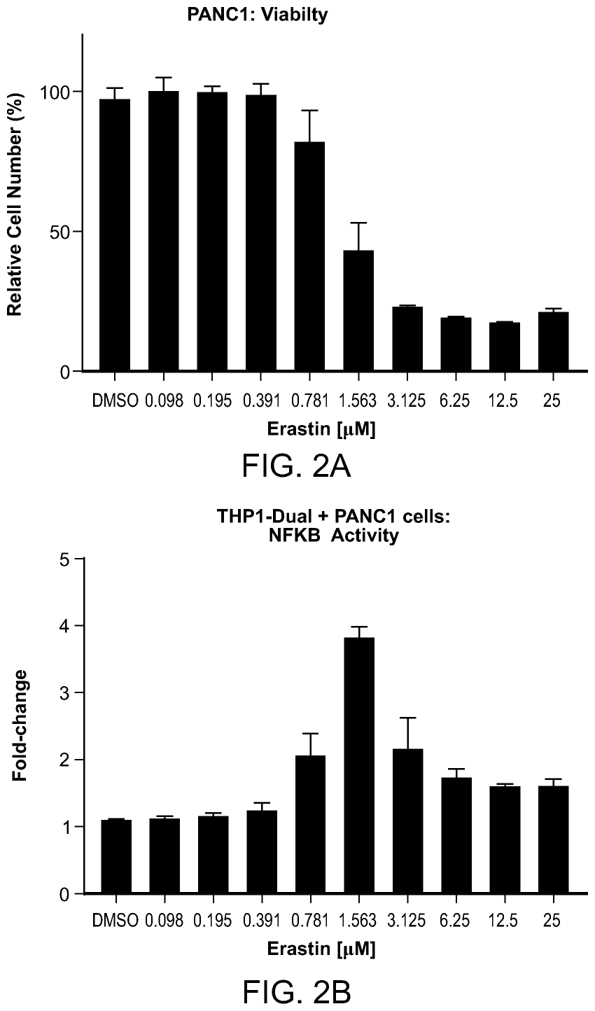

In some embodiments, an agent that induces iron-dependent cellular disassembly, e.g., ferroptosis, and is useful in the methods for identifying immunoinhibitory agents that inhibit iron-dependent cellular disassembly provided herein is an inhibitor of antiporter system Xc−. Inhibitors of antiporter system Xc− include antiporter system Xc− binding proteins (e.g., antibodies or antibody fragments), nucleic acid inhibitors (e.g., antisense oligonucleotides, or siRNAs), and small molecules that specifically inhibit antiporter system Xc−. For example, in some embodiments, the inhibitor of antiporter system Xc− is a binding protein, e.g., antibody or antibody fragment, that specifically inhibits SLC7A11 or SLC3A2. In some embodiments, the inhibitor of antiporter system Xc− is a nucleic acid inhibitor that specifically inhibits SLC7A11 or SLC3A2. In some embodiments, the inhibitor of antiporter system Xc− is small molecule that specifically inhibits SLC7A11 or SLC3A2. Antibody and nucleic acid inhibitors are well known in the art and are described in detail herein. Small molecule inhibitors of antiporter system Xc− include, but are not limited to, erastin, sulfasalazine, sorafenib, and analogs or derivatives thereof. (See Cao et al., 2016, Cell Mol Life Sci 73: 2195-2209, e.g., FIG. 2, incorporated in its entirety herein).

In a particular embodiment, an agent that induces iron-dependent cellular disassembly, e.g., ferroptosis, is erastin or an analog or derivative thereof. Analogs of erastin include, but are not limited to, the compounds listed in Table 1 below. Each of the references listed in Table 1 is incorporated by reference herein in its entirety.

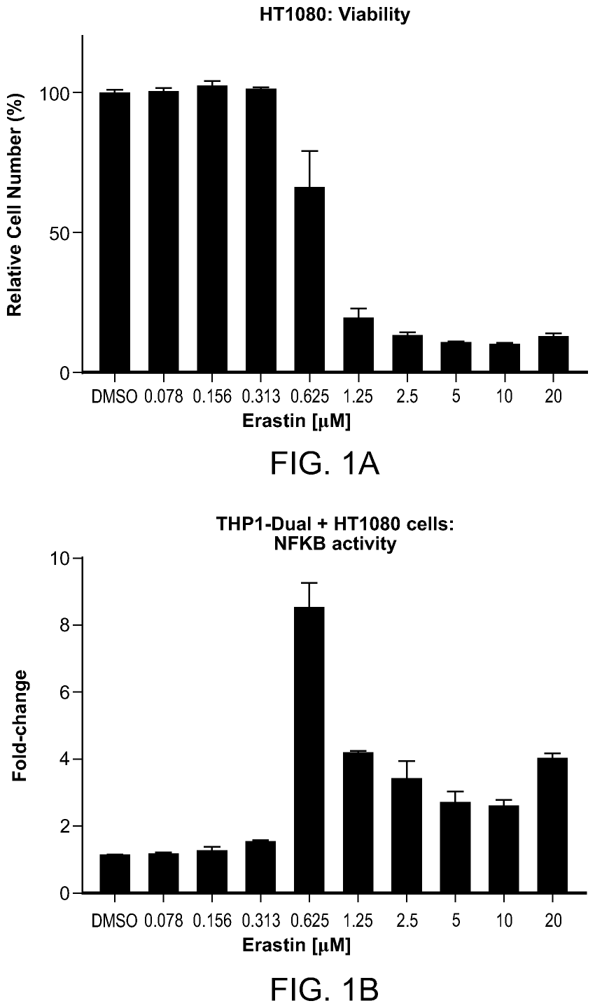

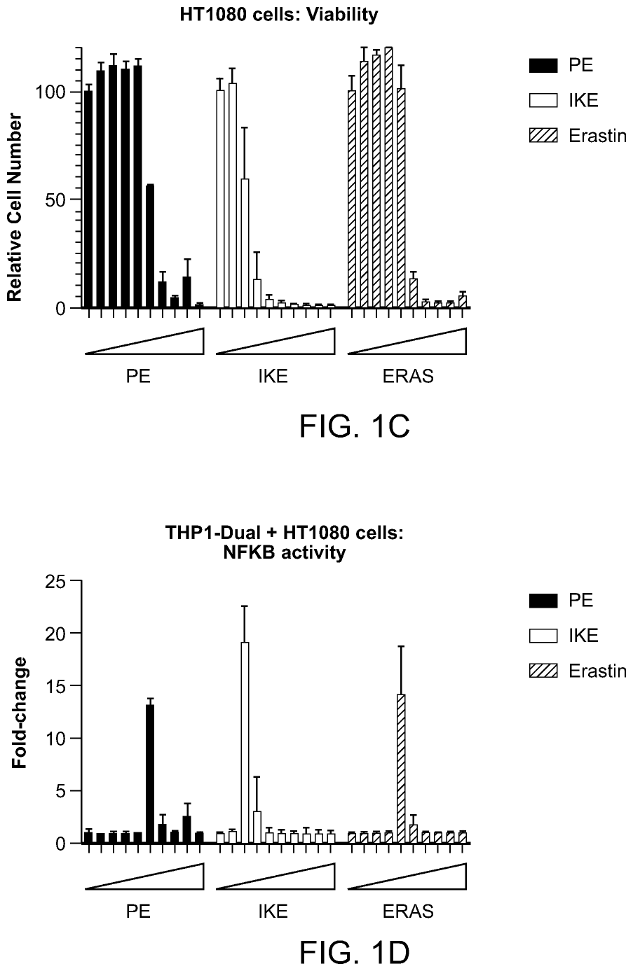

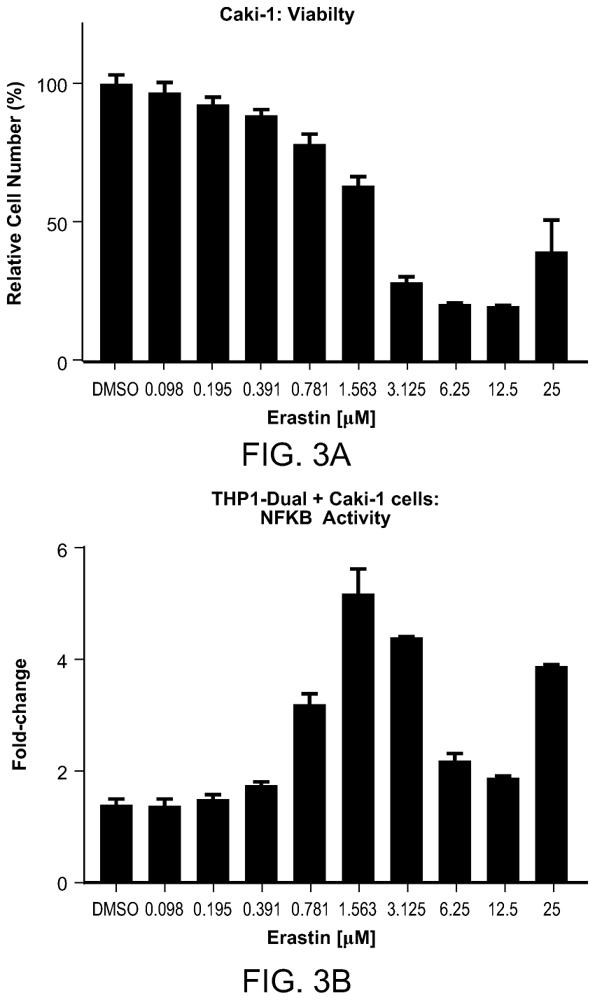

TABLE 1
Erastin Analogs

| Compound | Reference |
|---|---|
| PE | Yang et al., 2014, Cell 156: 317-331; FIG. 1C |
| AE | Yang et al., 2014, Cell 156: 317-331; FIG. 1C |
| IKE | Larraufie, et al., 2015, Bioorg MedChemLetters |
| | 25(21): 4787-4792; FIG. 3 |
| PKE | Larraufie, et al., 2015, Bioorg MedChemLetters |
| | 25(21): 4787-4792; FIG. 3 |
| 35MEW28, 21 | Dixon et al. 2014, eLife Feb 25;4. doi: |
| | 10.7554/eLife.05608; FIG. 3B/C |

In some embodiments, an agent that induces iron-dependent cellular disassembly (e.g., ferroptosis) and is useful in the methods for identifying immunoinhibitory agents that inhibit iron-dependent cellular disassembly provided herein is an inhibitor of glutathione peroxidase 4 (GPX4). GPX4 is a phospholipid hydroperoxidase that catalyzes the reduction of hydrogen peroxide and organic peroxides, thereby protecting cells against membrane lipid peroxidation, or oxidative stress. Thus, GPX4 contributes to a cell's ability to survive in oxidative environments. Inhibition of GPX4 can induce cell death by ferroptosis (see, Yang, W. S., et al. Regulation of ferroptotic cancer cell death by GPX4. Cell 156, 317-331 (2014)). Inhibitors of GPX4 include GPX4-binding proteins (e.g., antibodies or antibody fragments), nucleic acid inhibitors (e.g., antisense oligonucleotides or siRNAs), and small molecules that specifically inhibit GPX4. Small molecule inhibitors of GPX4 include, but are not limited to, the compounds listed in Table 2 below. Each of the references listed in Table 2 is incorporated by reference herein in its entirety.

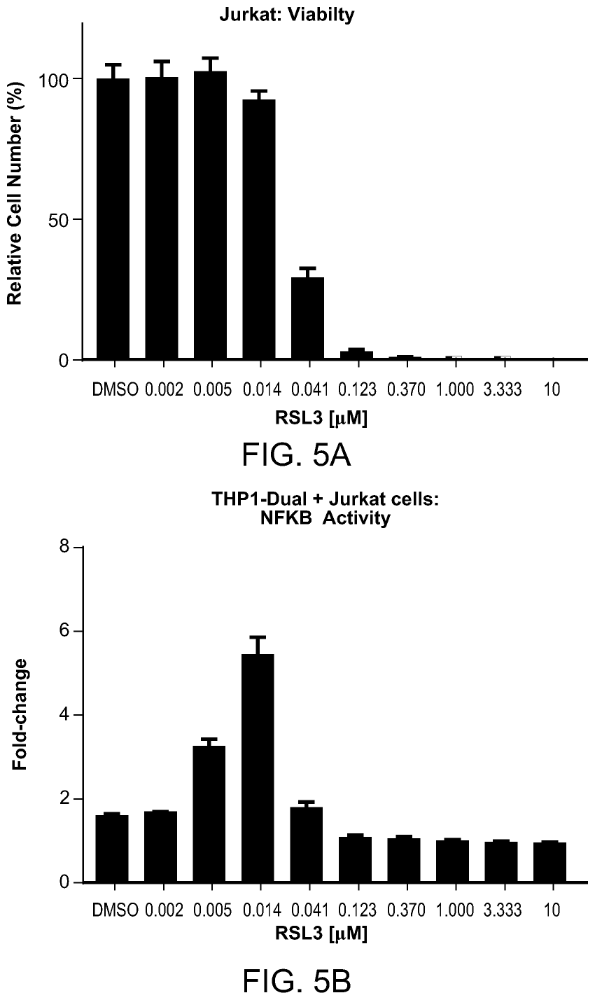

TABLE 2
GPX4 inhibitors

| Compound | Reference |
|---|---|
| DPI7 (ML162), | Yang et al., 2014, Cell 156: 317-331; FIGS. 5 |
| DPI19, DPI17, | and S5 |
| DPI13, DPI12 | |
| DPI10 (ML210) | Cao et al., 2016, Cell Mol Life Sci 73: 2195-2209; |
| | FIG. 2 |
| RSL3, or a derivative | Cao et al., 2016, Cell Mol Life Sci 73: 2195-2209; |
| or analog thereof | FIG. 2 |
| altretamine | Cao et al., 2016, Cell Mol Life Sci 73: 2195-2209; |
| | FIG. 2 |
| FIN56 | Shimada et al., 2016, Nat. Chem Biol. 12(7): |
| | 497-503 |
| FINO2 | Gaschler et al., 2018, Nature Chemical Biology |
| | 14:507-515 |

In a particular embodiment, the GPX4 inhibitor is

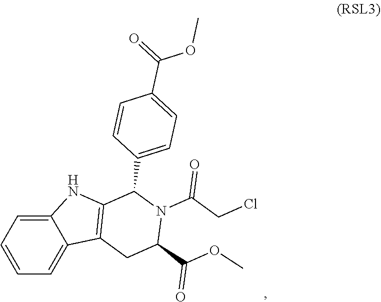 [SMILES: COC(=O)c1ccc([C@H]2c3[nH]c4ccccc4c3C[C@H](C(=O)OC)N2C(=O)CCl)cc1]

or a pharmaceutically acceptable salt thereof.

RSL3 is a known inhibitor of GPX4. In knockdown studies, RSL3 selectively mediated the death of RAS-expressing cells and was identified as increasing lipid ROS accumulation. See U.S. Pat. No. 8,546,421.

In some embodiments, the inhibitor of GPX4 is a diastereoisomer of RSL3.

In a particular embodiment, the diastereoisomer of RSL3 is

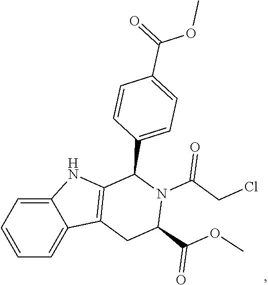 [SMILES: COC(=O)c1ccc([C@@H]2c3[nH]c4ccccc4c3C[C@H](C(=O)OC)N2C(=O)CCl)cc1]

or a pharmaceutically acceptable salt thereof.

In a particular embodiment, the diastereoisomer of RSL3 is

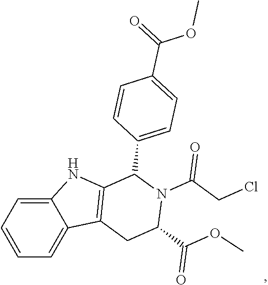

or a pharmaceutically acceptable salt thereof.

In a particular embodiment, the diastereoisomer of RSL3 is

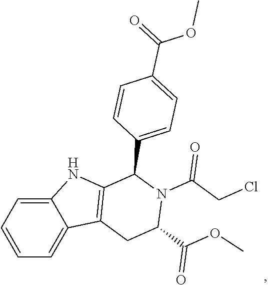 [SMILES: COC(=O)c1ccc([C@@H]2c3[nH]c4ccccc4c3C[C@@H](C(=O)OC)N2C(=O)CCl)cc1]

or a pharmaceutically acceptable salt thereof.

In some embodiments, the inhibitor of GPX4 is a pharmaceutically acceptable form of RSL3, including, but not limited to, N-oxides, crystalline form, hydrates, salts, esters, and prodrugs thereof.

In some embodiments, the inhibitor of GPX4 is RSL3 or a derivative or analog thereof. Derivatives and analogs of RSL3 are known in the art and are described, for example, in WO2008/103470, WO2017/120445, WO2018118711, U.S. Pat. No. 8,546,421, and CN108409737, each of which is incorporated by reference herein in its entirety.

In some embodiments, the RSL3 derivative or analog is a compound represented by Structural Formula (I):

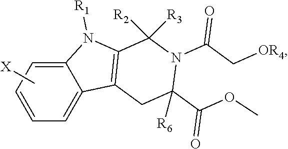

or an enantiomer, optical isomer, diastereomer, N-oxide, crystalline form, hydrate, or pharmaceutically acceptable salt thereof, wherein

R₁, R₂, R₃, and R₆ are independently selected from H, C₁₋₈alkyl, C₁₋₈alkoxy, C₁₋₈aralkyl, 3- to 8-membered carbocyclic, 3- to 8-membered heterocyclic, 3- to 8-membered aryl, or 3- to 8-membered heteroaryl, acyl, alkylsulfonyl, and arylsulfonyl, wherein each alkyl, alkoxy, aralkyl, carbocyclic, heterocyclic, aryl, heteroaryl, acyl, alkylsulfonyl, and arylsulfonyl is optionally substituted with at least one substituent;

R₄ and R₅ are independently selected from H₁C₁₋₈alkyl, C₁₋₈alkoxy, 3- to 8-membered carbocyclic, 3- to 8-membered heterocyclic, 3- to 8-membered aryl, or 3- to 8-membered heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether, wherein each alkyl, alkoxy, carbocyclic, heterocyclic, aryl, heteroaryl, carboxylate, ester, amide, carbohydrate, amino acid, acyl, alkoxy-substituted acyl, alditol, NR⁷R⁸, OC(R⁷)₂COOH, SC(R⁷)₂COOH, NHCHR⁷COOH, COR⁸, CO₂R⁸, sulfate, sulfonamide, sulfoxide, sulfonate, sulfone, thioalkyl, thioester, and thioether is optionally substituted with at least one substituent;

R⁷ is selected from H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;

R⁸ is selected from H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; and

X is 0-4 substituents on the ring to which it is attached.

In one embodiment, the RSL3 derivative or analog is a compound represented by Structural Formula (II):

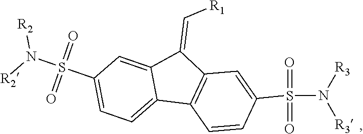

or an N-oxide, crystalline form, hydrate, or pharmaceutically acceptable salt thereof; wherein:

R₁ is selected from the group consisting of H, OH, and —(OCH₂CH₂)ₓOH;

X is an integer from 1 to 6; and

R₂, R₂′, R₃, and R₃′ independently are selected from the group consisting of H, C₃₋₈cycloalkyl, and combinations thereof, or R₂ and R₂′ may be joined together to form a pyridinyl or pyranyl and R₃ and R₃′ may be joined together to form a pyridinyl or pyranyl.

In one embodiment, the RSL3 derivative or analog is a compound represented by Structural Formula (III):

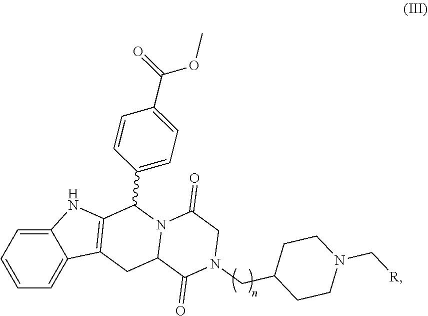

or a stereoisomer thereof, or a pharmaceutically acceptable salt thereof; wherein: n is 2, 3 or 4; and R is a substituted or unsubstituted C₁-C₆ alkyl group, a substituted or unsubstituted C₃-C₁₀ cycloalkyl group, a substituted or unsubstituted C₂-C₈ heterocycloalkyl group, a substituted or unsubstituted C₆-C₁₀ aromatic ring group, or a substituted or unsubstituted C₃-C₈ heteroaryl ring group; wherein the substitution means that one or more hydrogen atoms in each group are substituted by the following groups selected from the group consisting of: halogen, cyano, nitro, hydroxy, C₁-C₆ alkyl, halogenated C₁-C₆ alkyl, C₁-C₆ alkoxy, halogenated C₁-C₆ alkoxy, COOH (carboxy), COOC₁-C₆ alkyl, OCOC₁-C₆ alkyl.

In some embodiments, the GPX4 inhibitor is

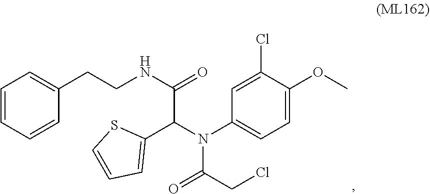 [SMILES: COc1ccc(N(C(=O)CCl)C(C(=O)NCCc2ccccc2)c2cccs2)cc1Cl]

or a pharmaceutically acceptable salt thereof.

ML162 has been identified as a direct inhibitor of GPX4 that induces ferroptosis (see, Dixon et al., 2015, ACS Chem. Bio. 10, 1604-1609).

In some embodiments, the GPX4 inhibitor is a pharmaceutically acceptable form of ML162, including, but not limited to, N-oxides, crystalline form, hydrates, salts, esters, and prodrugs thereof.

In some embodiments, the inhibitor of GPX4 is ML162 or a derivative or analog thereof.

In some embodiments, the GPX4 inhibitor is

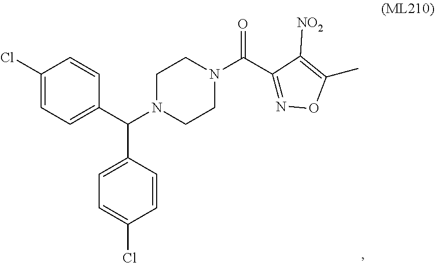 [SMILES: Cc1onc(C(=O)N2CCN(C(c3ccc(Cl)cc3)c3ccc(Cl)cc3)CC2)c1[N+](=O)[O-]]

or a pharmaceutically acceptable salt thereof.

In some embodiments, the GPX4 inhibitor is a pharmaceutically acceptable form of ML210, including, but not limited to, N-oxides, crystalline form, hydrates, salts, esters, and prodrugs thereof.

In some embodiments, the inhibitor of GPX4 is ML210 or a derivative or analog thereof.

In some embodiments, an agent that induces iron-dependent cellular disassembly (e.g., ferroptosis) and is useful in the methods for identifying immunoinhibitory agents that inhibit iron-dependent cellular disassembly provided herein is a statin. In one embodiment, the statin is selected from the group consisting of atorvastatin, fluvastatin, lovastatin, pitavastatin, pravastatin, rosuvastatin, cerivastatin and simvastatin.

In one embodiment, the agent that induces iron-dependent cellular disassembly (e.g., ferroptosis) and is useful in the methods for identifying immunoinhibitory agents that inhibit iron-dependent cellular disassembly provided herein is selected from the group consisting of glutamate, BSO, DPI2 (See Yang et al., 2014, Cell 156: 317-331; FIGS. 5 and S5, incorporated in its entirety herein), cisplatin, cysteinase, silica based nanoparticles, CCI4, ferric ammonium citrate, trigonelline and brusatol.

Additional agents that induce iron-dependent cellular disassembly are known in the art and are described, for example in U.S. Pat. Nos. 8,518,959; 8,535,897; 8,546,421; 9,580,398; 9,695,133; US2010/0081654; US2015/0079035; US2015/0175558; US2016/0229836; US2016/0297748; US2016/0332974; Cell. 2012 May 25; 149(5): 1060-72. doi: 10.1016/j.cell.2012.03.042;
Cell. 2014 Jan. 16; 156(1-2):317-331. doi: 10.1016/j.cell.2013.12.010;
J Am Chem Soc. 2014 Mar. 26; 136(12):4551-6. doi: 10.1021/ja411006a;
Elife. 2014 May 20; 3:e02523. doi: 10.7554/eLife.02523;
Proc Natl Acad Sci USA. 2014 Nov. 25; 111(47): 16836-41. doi: 10.1073/pnas.1415518111;
Nat Cell Biol. 2014 December; 16(12):1180-91. doi: 10.1038/ncb3064;
ACS Chem Biol. 2015 Jul. 17; 10(7):1604-9. doi: 10.1021/acschembio.5b00245;
Bioorg Med Chem Lett. 2015 Nov. 1; 25(21):4787-92. doi: 10.1016/j.bmcl.2015.07.018;
Nat Rev Drug Discov. 2016 May; 15(5):348-66. doi: 10.1038/nrd.2015.6;
Cell Chem Biol. 2016 Feb. 18; 23(2):225-235. doi:10.1016/j.chembiol.2015.11.016;
Nat Chem Biol. 2016 July; 12(7):497-503. doi: 10.1038/nchembio.2079;
Proc Natl Acad Sci USA. 2016 Aug. 23; 113(34):E4966-75. doi: 10.1073/pnas.1603244113;
ACS Cent Sci. 2016 Sep. 28; 2(9):653-659;
Nat Chem Biol. 2017 January; 13(1):81-90. doi: 10.1038/nchembio.2238;
Biochem Biophys Res Commun. 2017 Jan. 15; 482(3):419-425. doi:
10.1016/j.bbrc.2016.10.086; Nat Chem Biol. 2018 May; 14(5):507-515. doi: 10.1038/s41589-018-0031-6;
Cancer Lett. 2018 Apr. 24. pii: S0304-3835(18)30288-X. doi: 10.1016/j.canlet.2018.04.021;
J Med Chem. 2018 Apr. 24. doi: 10.1021/acs.jmedchem.8b00315;
Eur J Med Chem. 2018 May 10; 151:434-449. doi: 10.1016/j.ejmech.2018.04.005;
Neuropharmacology. 2018 Mar. 15; 135:242-252. doi: 10.1016/j.neuropharm.2018.03.015;
J Am Chem Soc. 2018 Mar. 14; 140(10):3798-3808. doi: 10.1021/jacs.8b00998;
Biochem Biophys Res Commun. 2018 Feb. 26; 497(1):233-240. doi: 10.1016/j.bbrc.2018.02.061;
Org Biomol Chem. 2018 Feb. 28; 16(9):1465-1479. doi: 10.1039/c7ob03086j;
Arch Toxicol. 2018 April; 92(4): 1507-1524. doi: 10.1007/s00204-018-2170-7;
Int J Oncol. 2018 March; 52(3): 1011-1022. doi: 10.3892/ijo.2018.4259;
Cancer Lett. 2018 Apr. 28; 420:210-227. doi: 10.1016/j.canlet.2018.01.061. February 1;
Free Radic Biol Med. 2018 March; 117:45-57. doi: 10.1016/j.freeradbiomed.2018.01.019;
Sci Rep. 2018 Jan. 12; 8(1):574. doi: 10.1038/s41598-017-18935-1;
Cancer Lett. 2018 Mar. 1; 416:124-137. doi: 10.1016/j.canlet.2017.12.025;
Chem Med Chem. 2018 Jan. 22; 13(2):164-177. doi: 10.1002/cmdc.201700629;
Redox Biol. 2018 April; 14:535-548. doi: 10.1016/j.redox.2017.11.001;
Arch Toxicol. 2018 February; 92(2):759-775. doi: 10.1007/s00204-017-2066-y;
Nat Chem. 2017 October; 9(10): 1025-1033. doi: 10.1038/nchem.2778;
Free Radic Biol Med. 2017 November; 112:597-607. doi: 10.1016/j.freeradbiomed.2017.09.002;
ACS Chem Biol. 2017 Oct. 20; 12(10):2538-2545. doi: 10.1021/acschembio.7b00730. 20;
PLoS One. 2017 Aug. 21; 12(8):e0182921. doi: 10.1371/joumal.pone.0182921. eCollection 2017; Biochem Biophys Res Commun. 2017 Sep. 30; 491(4):919-925. doi: 10.1016/j.bbrc.2017.07.136;
Biochem Pharmacol. 2017 Sep. 15; 140:41-52. doi: 10.1016/j.bcp.2017.06.112. 23;
Cancer Res Treat. 2018 April; 50(2):445-460. doi: 10.4143/crt.2016.572;
Cell Death Discov. 2017 Feb. 27; 3:17013. doi: 10.1038/cddiscovery.2017.13. eCollection 2017; Nat Nanotechnol. 2016 November; 11(11):977-985. doi: 10.1038/nnano.2016.164;
Biochem Biophys Res Commun. 2016 Nov. 25; 480(4):602-607. doi: 10.1016/j.bbrc.2016.10.099; Oncotarget. 2016 Nov. 15; 7(46):74630-74647. doi: 10.18632/oncotarget. 11858;
Cancer Lett. 2016 Oct. 10; 381(1): 165-75. doi: 10.1016/j.canlet.2016.07.033. 29;
Cell Death Dis. 2016 Jul. 21; 7:e2307. doi:10.1038/cddis.2016.208;
Oncol Rep. 2016 August; 36(2):968-76. doi: 10.3892/or.2016.4867. 31;
Biochem Biophys Res Commun. 2016 May 13; 473(4):775-780. doi: 10.1016/j.bbrc.2016.03.052; Mol Carcinog. 2017 January; 56(1):75-93. doi: 10.1002/mc.22474;
ACS Chem Biol. 2016 May 20; 11(5): 1305-12. doi: 10.1021/acschembio.5b00900;
J Med Chem. 2016 Mar. 10; 59(5):2041-53. doi: 10.1021/acs.jmedchem.5b01641;
Phytomedicine. 2015 Oct. 15; 22(11): 1045-54. doi: 10.1016/j.phymed.2015.08.002;
Biol Pharm Bull. 2015; 38(8): 1234-9. doi: 10.1248/bpb.b15-00048;
Oncoscience. 2015 May 2; 2(5):517-32. eCollection 2015;
Pathol Oncol Res. 2015 September; 21(4):1115-21. doi: 10.1007/s12253-015-9946-3;
Anticancer Res. 2014 November; 34(11):6417-22; and
Int J Cancer. 2013 Oct. 1; 133(7): 1732-42. doi: 10.1002/ijc.28159;
   each of which is incorporated by reference herein in its entirety.

### EXAMPLES

### Example 1: Activation/Stimulation of Human Monocytes by HT1080 Fibrosarcoma Cells Treated with Erastin

### Agent/Therapeutic Design:

HT1080 fibrosarcoma cells were treated with either control (DMSO) or various doses of Erastin, piperazine erastin (PE), or imidazole ketoerastin (IKE) for 24 hours prior to co-culture with THP1-Dual cells for an additional 24 hours. Erastin was purchased from Selleckchem (Houston, Tex.) and dissolved in DMSO. Subsequently, the THP1 supernatants were assessed for nuclear factor kappa-light-chain-enhancer of activated B cells (NFKB) or interferon regulatory factor (IRF) reporter activity.

### Materials/Methods:

HT1080 cells were acquired from ATCC and THP1-Dual cells were acquired from InvivoGen (San Diego, Calif.). THP1-Dual cells are human monocyte cells that induce reporter proteins upon activation of either NFKB or IRF pathways. Both cell types were cultured in 96-well plates for the duration of the assay. HT1080 cells were cultured in DMEM with 10% FBS, and THP1-Dual cells were cultured in RPMI with 10% FBS. 7,500 HT1080 cells were plated 24 hours prior to dosing with Erastin, PE, or IKE with a final DMSO concentration of 0.5%. 24 hours post-treatment, THP1-Dual cells (25,000 cells/well) were added to the HT1080 cells. 24 hours later, 30 μl of supernatant was mixed with either 200 μl QuantiBlue (InvivoGen, San Diego, Calif.) (for NFKB reporter activity) or 50 μl QuantiLuc (for IRF reporter activity) and absorbance or luminescence was recorded on a Molecular Devices plate reader.

### Conclusion

As shown in FIG. 1A, Erastin treatment negatively affected the viability of HT1080 cells. FIG. 1B shows that HT1080 cells treated with Erastin, but not the vehicle control DMSO, elicited NFKB signaling in THP1 monocytes. The erastin analogs PE and IKE also negatively affected the viability of HT1080 cells (FIG. 1C) and elicited NFKB signaling in THP1 monocytes (FIG. 1D).

### Example 2: Activation/Stimulation of Human Monocytes by PANC1 Pancreatic Cancer Cells Treated with Erastin

### Agent/Therapeutic Design:

PANC1 pancreatic cancer cells were treated with either control (DMSO) or various doses of Erastin, for 24 hours prior to co-culture with THP1-Dual cells for an additional 24 hours. Erastin was purchased from Selleckchem (Houston, Tex.) and dissolved in DMSO. Subsequently, the THP1 supernatants were assessed for NFKB or IRF reporter activity.

### Materials/Methods:

PANC1 cells were acquired from ATCC and THP1-Dual cells were acquired from InvivoGen (San Diego, Calif.). Both cell types were cultured in 96-well plates for the duration of the assay. 7,500 PANC-1 cells were plated 24 hours prior to dosing with Erastin, with a final DMSO concentration of 0.5%. 24 hours post-treatment, THP1-Dual cells (25,000 cells/well) were added to the PANC-1 cells. 24 hours later, 30 μl of supernatant was mixed with either 200 μl QuantiBlue (InvivoGen, San Diego, Calif.) or 50 μl QuantiLuc and absorbance or luminescence was recorded on a Molecular Devices plate reader.

### Conclusion

As shown in FIG. 2A, Erastin treatment negatively affected the viability of PANC1 cells. FIG. 1B shows that PANC1 cells treated with Erastin, but not the vehicle control DMSO, elicited NFKB signaling in THP1 monocytes.

### Example 3: Activation/Stimulation of Human Monocytes by Caki-1 Renal Cell Carcinoma Cells Treated with Erastin

### Agent/Therapeutic Design:

Caki-1 renal cell carcinoma cells were treated with either control (DMSO) or various doses of Erastin, for 24 hours prior to co-culture with THP1-Dual cells for an additional 24 hours. Erastin was purchased from Selleckchem (Houston, Tex.) and dissolved in DMSO. Subsequently, the THP1 supernatants were assessed for NFKB or IRF reporter activity.

### Materials/Methods:

Caki-1 cells were acquired from ATCC and THP1-Dual cells acquired from InvivoGen (San Diego, Calif.). Both cell types were cultured in 96-well plates for the duration of the assay. 7.500 Caki-1 cells were plated 24 hours prior to dosing with Erastin, with a final DMSO concentration of 0.5%. 24 hours post-treatment, THP1-Dual cells (25,000 cells/well) were added to the Caki-1 cells. 24 hours later, 30 μl of supernatant was mixed with either 200 μl QuantiBlue (InvivoGen) or 50 μl QuantiLuc and absorbance or luminescence was recorded on a Molecular Devices plate reader.

### Conclusion

As shown in FIG. 3A, Erastin treatment negatively affected the viability of Caki-1 cells. FIG. 3B shows that Caki-1 cells treated with Erastin, but not the vehicle control DMSO, elicited NFKB signaling in THP1 monocytes.

### Example 4: Activation/Stimulation of Human Monocytes by Caki-1 Renal Cell Carcinoma Cells Treated with RSL3

### Agent/Therapeutic Design:

Caki-1 renal cell carcinoma cells were treated with either control (DMSO) or various doses of RSL3, for 24 hours prior to co-culture with THP1-Dual cells for an additional 24 hours. RSL3 was purchased from Selleckchem and dissolved in DMSO. Subsequently, the THP1 supernatants were assessed for NFKB or IRF reporter activity.

### Materials/Methods:

Caki-1 cells were acquired from ATCC and THP1-Dual cells acquired from InvivoGen (San Diego, Calif.). Both cell types were cultured in 96-well plates for the duration of the assay. 7.500 Caki-1 cells were plated 24 hours prior to dosing with RSF3, with a final DMSO concentration of 0.5%. 24 hours post-treatment, THP1-Dual cells (25,000 cells/well) were added to the Caki-1 cells. 24 hours later, 30 μl of supernatant was mixed with either 200 μl QuantiBlue (InvivoGen, San Diego, Calif.) or 50 μl QuantiFuc and absorbance or luminescence was recorded on a Molecular Devices plate reader.

### Conclusion

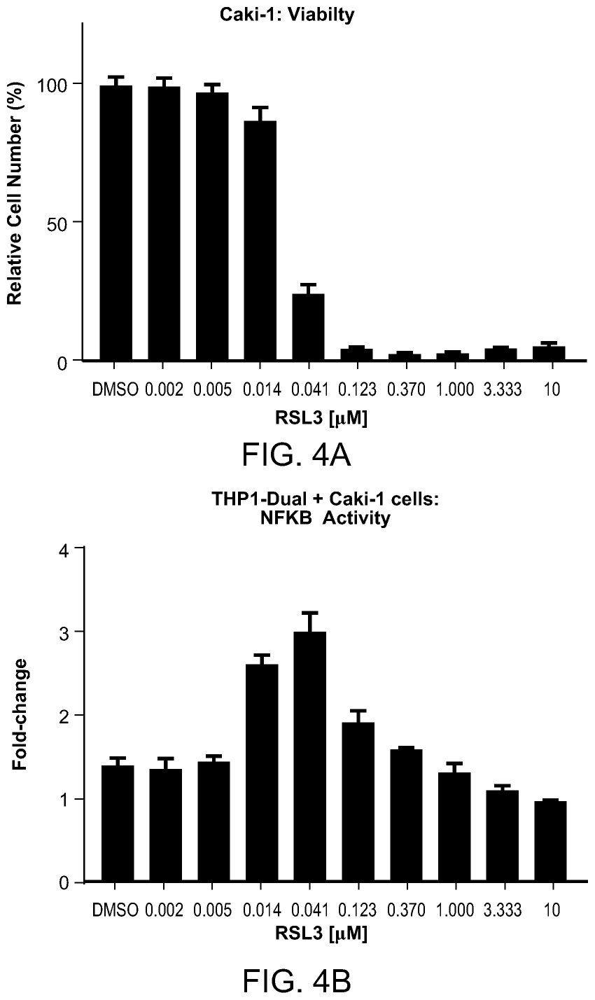

As shown in FIG. 4A, RSL3 treatment negatively affected the viability of Caki-1 cells. FIG. 4B shows that Caki-1 cells treated with RSL3, but not the vehicle control DMSO, elicited NFKB signaling in THP1 monocytes.

### Example 5: Activation/Stimulation of Human Monocytes by Jurkat T Cell Leukemia Cells Treated with RSL3

### Agent/Therapeutic Design:

Jurkat T cell leukemia cells were treated with either control (DMSO) or various doses of RSL3, for 24 hours prior to co-culture with THP1-Dual cells for an additional 24 hours. RSL3 was purchased from Selleckchem (Houston, Tex.) and dissolved in DMSO. Subsequently, the THP1 supernatants were assessed for NFKB or IRF reporter activity.

### Materials/Methods:

Jurkat cells were acquired from ATCC and THP1-Dual cells acquired from InvivoGen (San Diego, Calif.). Both cell types were cultured in 96-well plates for the duration of the assay. 100,000 Jurkat cells were plated 24 hours prior to dosing with RSL3, with a final DMSO concentration of 0.5%. 24 hours post-treatment, THP1-Dual cells (25,000 cells/well) were added to the Jurkat cells. 24 hours later, 30 μl of supernatant was mixed with either 200 μl QuantiBlue (InvivoGen, San Diego, Calif.) or 50 μl QuantiLuc and absorbance or luminescence was recorded on a Molecular Devices plate reader.

### Conclusion

As shown in FIG. 5A, RSL3 treatment negatively affected the viability of Jurkat T cell leukemia cells. FIG. 5B shows that Jurkat cells treated with RSL3, but not the vehicle control DMSO, elicited NFKB signaling in THP1 monocytes.

### Example 6: Activation/Stimulation of Human Monocytes by A20 B-Cell Leukemia Cells Treated with RSL3

### Agent/Therapeutic Design:

A20 B-cell leukemia cells were treated with either control (DMSO) or various doses of RSL3, for 24 hours prior to co-culture with THP1-Dual cells for an additional 24 hours. RSL3 was purchased from Selleckchem (Houston, Tex.) and dissolved in DMSO. Subsequently, the THP1 supernatants were assessed for NFKB or IRF reporter activity.

### Materials/Methods:

A20 cells were acquired from ATCC and THP1-Dual cells acquired from InvivoGen (San Diego, Calif.). Both cell types were cultured in 96-well plates for the duration of the assay. 50,000 A20 cells were plated 24 hours prior to dosing with RSL3, with a final DMSO concentration of 0.5%. 24 hours post-treatment, THP1-Dual cells (25,000 cells/well) were added to the A20 cells. 24 hours later, 30 μl of supernatant was mixed with either 200 μl QuantiBlue (InvivoGen, San Diego, Calif.) or 50 μl QuantiLuc and absorbance or luminescence was recorded on a Molecular Devices plate reader.

### Conclusion

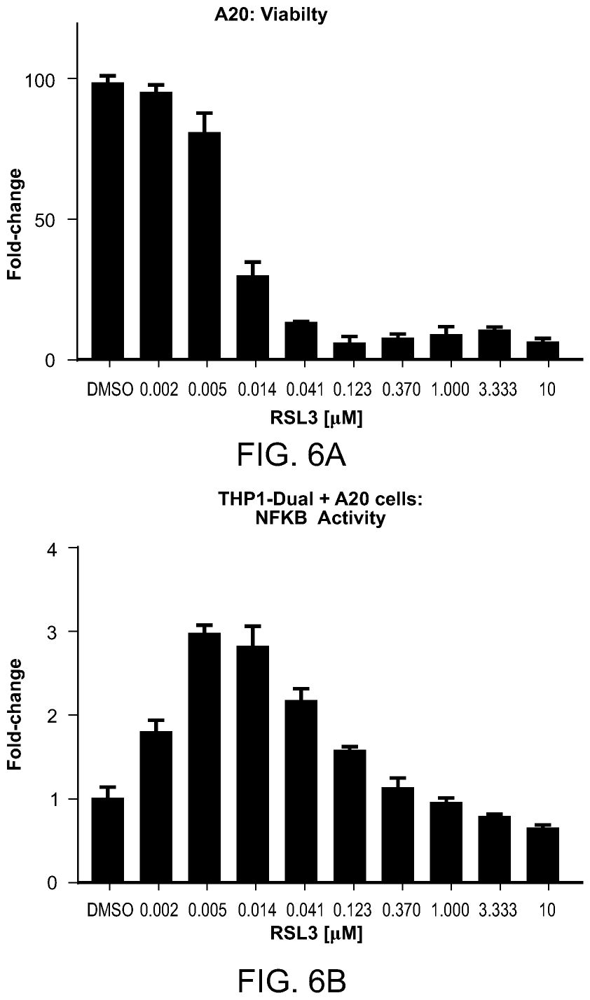

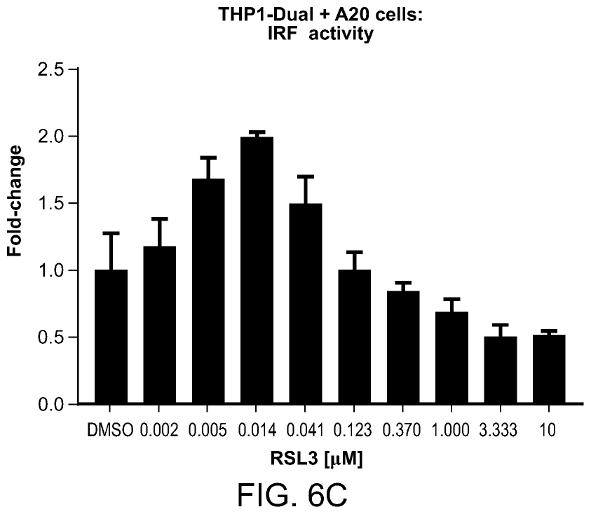

As shown in FIG. 6A, RSL3-treatment negatively affected the viability of A20 B cell leukemia cells. A20 cells treated with RSL3, but not the vehicle control DMSO, elicited NFKB (FIG. 6B) and IRF (FIG. 6C) signaling in THP1 monocytes.

### Example 7: Specificity of Pro-Inflammatory Signaling Elicited by HT1080 Fibrosarcoma Cells Treated with Erastin

### Agent/Therapeutic Design:

HT1080 fibrosarcoma cells are treated with either control (DMSO) or various doses of Erastin (e.g. 0.098, 0.195, 0.391, 0.781, 1.563, 3.125, 6.25, 12.5 and 25 μM) in the presence or absence of 1 μM Ferrostatin for 24 hours prior to co-culture with THP1-Dual cells for an additional 24 hours. Ferrostatin is purchased from Selleckchem (Houston, Tex.) and dissolved in DMSO. Subsequently, the THP1 supernatant is assessed for NFKB or IRF reporter activity. HT1080 cells are acquired from ATCC and THP1-Dual cells acquired from InvivoGen (San Diego, Calif.). Both cell types are cultured in 96-well plates for the duration of the assay. The specificity of induction of NFKB signaling elicited by Erastin-treated HT1080 cells is assessed by its reversal by concomitant ferrostatin treatment of HT1080 cells. Reversal of erastin-induced NFKB or IRF signaling with Ferrostatin would indicate that an inhibitor of iron-dependent cellular disassembly decreases immune response.

### Example 8: Specificity of Pro-Inflammatory Signaling Elicited by Caki-1 Cells Treated with Erastin

### Agent/Therapeutic Design:

Caki-1 renal carcinoma cells are treated with either control (DMSO) or various doses of Erastin (e.g. 0.098, 0.195, 0.391, 0.781, 1.563, 3.125, 6.25, 12.5 and 25 μM) in the presence or absence of 1 μM Ferrostatin (Selleckchem; Houston, Tex.) for 24 hours prior to co-culture with THP1-Dual cells for an additional 24 hours. Subsequently, the THP1 supernatant is assessed for NFKB or IRF reporter activity. Caki-1 cells are acquired from ATCC and THP1-Dual cells acquired from InvivoGen (San Diego, Calif.). Both cell types are cultured in 96-well plates for the duration of the assay. The specificity of induction of NFKB signaling elicited by Erastin-treated Caki-1 cells is assessed by its reversal by concomitant ferrostatin treatment of Caki-1 cells. Reversal of erastin-induced NFKB or IRF signaling with Ferrostatin would indicate that an inhibitor of iron-dependent cellular disassembly decreases immune response.

### Example 9:) Specificity of Pro-Inflammatory Signaling Elicited by Caki-1 Renal Carcinoma Cells Treated with RSL3

### Agent/Therapeutic Design:

Caki-1 renal carcinoma cells are treated with either control (DMSO) or various doses of RSF3 (e.g. 0.002, 0.005, 0.014, 0.041, 0.123, 0.370, 1.111, 3.333 and 10 μM) in the presence or absence of 1 μM Ferrostatin for 24 hours prior to co-culture with THP1-Dual cells for an additional 24 hours. Subsequently, the THP1 supernatant is assessed for NFKB or IRF reporter activity. Caki-1 cells are acquired from ATCC and THP1-Dual cells acquired from InvivoGen (San Diego, Calif.). Both cell types are cultured in 96-well plates for the duration of the assay. The specificity of induction of NFKB signaling elicited by RSF3-treated Caki-1 cells is assessed by its reversal by concomitant ferrostatin treatment of Caki-1 cells. Reversal of RSF3-induced NFKB or IRF signaling with Ferrostatin would indicate that an inhibitor of iron-dependent cellular disassembly decreases immune response.

### Example 10: Specificity of Pro-Inflammatory Signaling Elicited by A20 Cells Treated with RSL3

### Agent/Therapeutic Design:

A20 lymphoma cells are treated with either control (DMSO) or various doses of RSL3 (e.g. 0.002, 0.005, 0.014, 0.041, 0.123, 0.370, 1.111, 3.333 and 10 μM) in the presence or absence of 1 μM Ferrostatin for 24 hours prior to co-culture with THP1-Dual cells for an additional 24 hours. Subsequently, the THP1 supernatant is assessed for NFKB or IRF reporter activity. A20 cells are acquired from ATCC and THP1-Dual cells are acquired from InvivoGen (San Diego, Calif.). Both cell types are cultured in 96-well plates for the duration of the assay. The specificity of induction of NFKB signaling elicited by RSL3-treated A20 cells is assessed by its reversal by concomitant ferrostatin treatment of A20 cells.

### Example 11: Specificity of Pro-Inflammatory Signaling Elicited by Epithelial Cells Treated with Erastin

### Agent/Therapeutic Design:

Epithelial cells (e.g. primary renal proximal tubular epithelial cells or primary intestinal epithelial cells) are treated with either control (DMSO) or various doses of Erastin (e.g. 0.098, 0.195, 0.391, 0.781, 1.563, 3.125, 6.25, 12.5 and 25 μM) in the presence or absence of 1 μM Ferrostatin for 24 hours prior to co-culture with THP1-Dual cells for an additional 24 hours. Ferrostatin is purchased from Selleckchem (Houston, Tex.) and dissolved in DMSO. Subsequently, the THP1 supernatant is assessed for NFKB or IRF reporter activity. The epithelial cells are acquired from ATCC and THP1-Dual cells are acquired from InvivoGen (San Diego, Calif.). Both cell types are cultured in 96-well plates for the duration of the assay. The specificity of induction of NFKB or IRF signaling elicited by Erastin-treated epithelial cells is assessed by its reversal by concomitant ferrostatin treatment of the epithelial cells. In addition, inhibition of NFKB or IRF induction by ferrostatin would illustrate that an inhibitor of iron-dependent cellular disassembly can decrease immune response.

### Example 12: Specificity of Pro-Inflammatory Signaling Elicited by Epithelial Cells Treated with RSL3

### Agent/Therapeutic Design:

Epithelial cells (e.g. primary renal proximal tubular epithelial cells or primary intestinal epithelial cells) are treated with either control (DMSO) or various doses of or various doses of RSL3 (e.g. 0.002, 0.005, 0.014, 0.041, 0.123, 0.370, 1.111, 3.333 and 10 μM) in the presence or absence of 1 μM Ferrostatin for 24 hours prior to co-culture with THP1-Dual cells for an additional 24 hours. Ferrostatin is purchased from Selleckchem (Houston, Tex.) and dissolved in DMSO. Subsequently, the THP1 supernatant is assessed for NFKB or IRF reporter activity. The epithelial cells are acquired from ATCC and THP1-Dual cells are acquired from InvivoGen (San Diego, Calif.). Both cell types are cultured in 96-well plates for the duration of the assay. The specificity of induction of NFKB or IRF signaling elicited by RSF3-treated epithelial cells is assessed by its reversal by concomitant ferrostatin treatment of the epithelial cells. In addition, inhibition of NFKB or IRF induction by ferrostatin would illustrate that an inhibitor of iron-dependent cellular disassembly can decrease immune response.

### Example 13: Testing the Inhibition of Pro-Inflammatory Signaling by a Ferroptosis Inhibitor in an In Vivo Model of Injury and Regeneration

### Agent/Therapeutic Design:

C57/BF6 mice are treated with either vehicle or ferrostatin (10 mg/kg, i.p.) and subjected to a renal ischemia-reperfusion injury. Briefly, mice are exposed by flank incision and clamped for 60 minutes. After releasing the clamp, flank incisions are closed with sutures. Sham surgeries are performed in a similar manner but without clamping renal vessels. Inflammation of the outer medulla is assessed 2 days and 7 days post-surgery. The anti-inflammatory effect of ferrostatin is evaluated by monitoring for a decrease in macrophage and neutrophil recruitment as assessed by histology (F4/80 staining for macrophages and Napthol AS-D chloroacetate esterase staining) and/or flow-cytometry.

### Example 14: Testing the Inhibition of Ulcerative Colitis in Mice by a Ferroptosis Inhibitor

### Agent/Therapeutic Design:

C57/B6 mice are treated with vehicle or Ferrostatin (10 mg/kg, i.p) before the induction of colitis by administration of 3-4% dextran sodium sulfate in the drinking water for 5 days. An anti-inflammatory effect of ferrostatin compared to vehicle is confirmed by decreased colon length, decreased diarrhea, and decreased weight loss. In addition, inhibition of neutrophil recruitment (innate immune response) by ferrostatin in confirmed by decreased myeloperoxidase activity in the colon (compared to vehicle).

### Example 15: A Method of Screening for Compounds that Inhibit Pro-Inflammatory Ferroptosis

### Agent/Therapeutic Design:

Caki-1 renal carcinoma cells in 384-well format are exposed to Erastin or RSL3 (or other ferroptosis inducers) and are additionally exposed to compounds from a chemical screening library for 24 hours. Subsequently, THP1 dual cells are co-cultured with the treated Caki-1 cells. 24 hours post-addition of THP1-Dual cells, supernatants are assessed for reporter activity. Positive hit compounds are selected for their ability to inhibit the induction of NFKB or IRF activity in THP1 Dual cells caused by co-culture with the ferroptosis inducer-treated Caki-1 cells.

### Example 16: Specificity of Pro-Inflammatory Signaling Elicited by HT1080 Fibrosarcoma Cells Treated with Erastin

### Agent/Therapeutic Design:

HT1080 fibrosarcoma cells were treated with various doses of Erastin (e.g. 0.8, 0.16, 0.31, 0.63, 1.25, 2.5, 5, 10 or 20 μM) alone or in combination with a ferroptosis inhibitor (1 μM Ferrostatin-1, 1 μM Liproxstatin-1, 100 μM Trolox, 25 μM β-Mercaptoethanol or 100 μM Deferoxamine) for 24 hours prior to co-culture with THP1-Dual cells for an additional 24 hours. Ferrostatin-1 and Liproxstatin-1 were purchased from Selleckchem (Houston, Tex.) and dissolved in DMSO. Trolox was purchased from Cayman Chemical Company Inc and resuspended in DMSO. Deferoxamine mesylate was purchased from Sigma-Aldrich and resuspended in water. β-Mercaptoethanol was purchased from Life Technologies. Subsequently, the THP1 supernatant was assessed for NFKB activity. HT1080 cells were acquired from ATCC and THP1-Dual cells were acquired from InvivoGen (San Diego, Calif.). Both cell types were cultured in 96-well plates for the duration of the assay.

### Results:

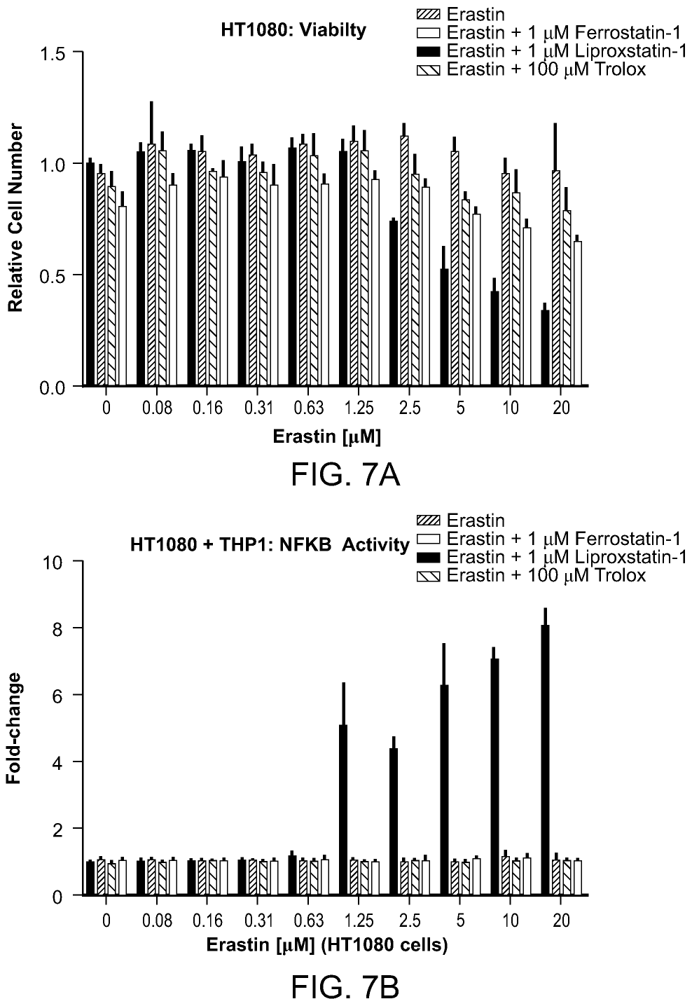

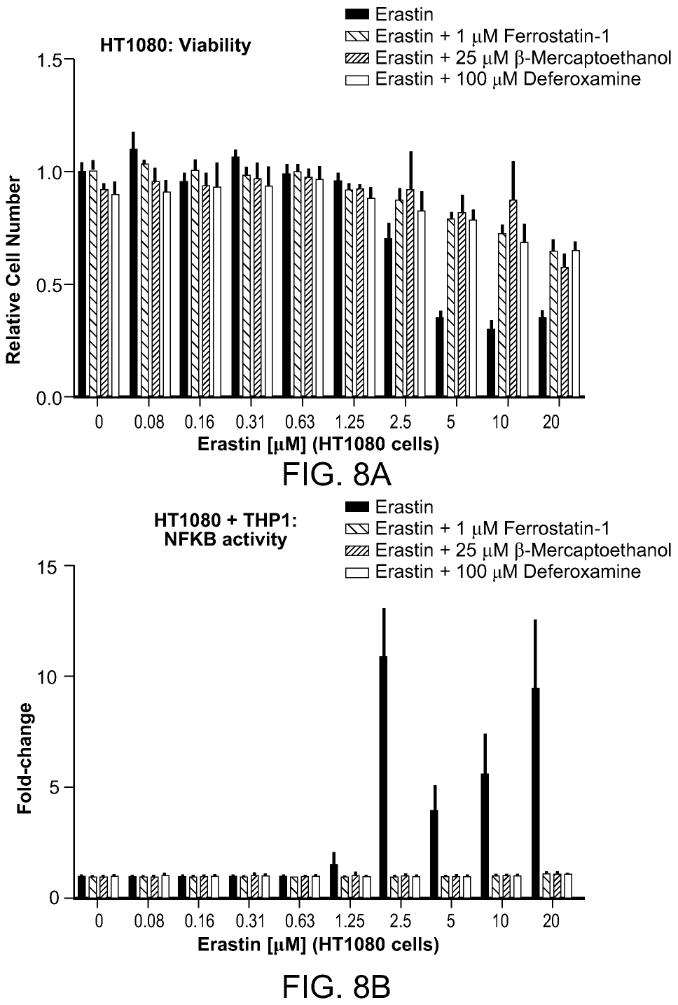

As shown in FIGS. 7A and 8A, Erastin treatment of HT1080 fibrosarcoma cells decreased viability of the cells in a dose dependent manner, and this decreased viability was attenuated by each of the ferroptosis inhibitors. As shown in FIGS. 7B and 8B, Erastin treatment of HT1080 fibrosarcoma cells increased NFKB activity in THP1 cells in a dose dependent manner, and this increased NFKB activity was abrogated by each of the ferroptosis inhibitors. These results demonstrate that cell death plays a role in the induction of NFKB signaling elicited by Erastin-treated HT1080 cells. In addition, because the ferroptosis inhibitors reduced NFKB activity induction by erastin-treated cells, these results demonstrate that inhibiting iron-dependent cellular disassembly (e.g., by ferroptosis inhibitors) can decrease immune activity.

### Example 17: Knockdown of ACSL4 and CARS Genes Inhibits Erastin-Mediated Cell Death in HT1080 Fibrosarcoma Cells

### Agent/Therapeutic Design:

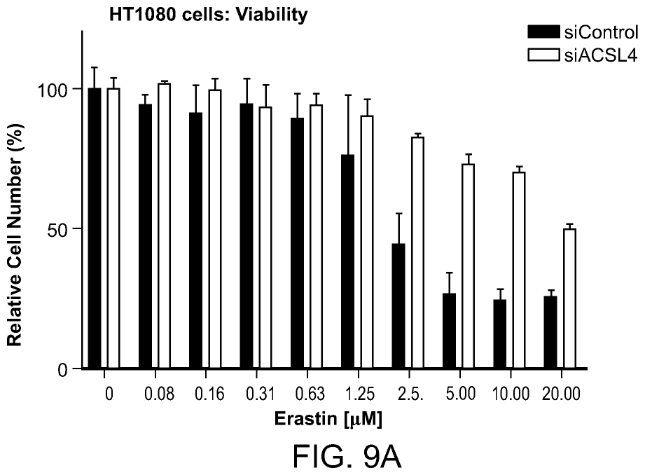

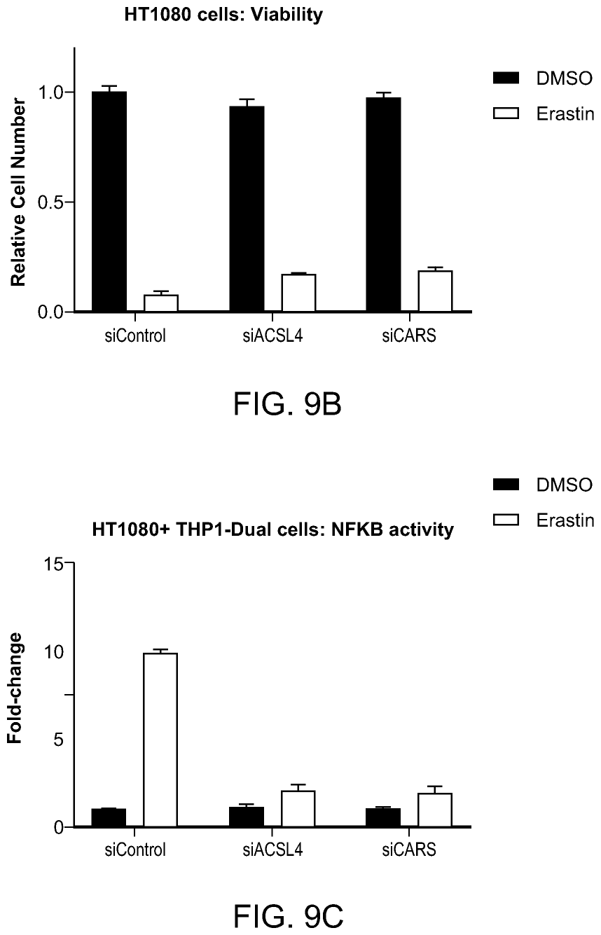

HT1080 cells (5,000 cells/well) were reverse transfected in 96-well format using DharmaFECT I transfection reagent (Catalog #T-2001) and control siRNA (Dharmacon Catalog #D-001810-10-05) or siRNA [37.5 nM] targeting ACSL4 (FIG. 9A, Dharmacon Catalog #L-009364-00-005) or a pool of siRNAs (FIG. 9B/C) against ACSL4 (Thermo Fisher Silencer Select Catalog #'s: s5001, s5001, s5002) or CARS (Thermo Fisher Silencer Select Catalog #'s: S2404, s2405, s2406). 48 hours post-transfection, cell culture medium was replaced by fresh medium containing various concentrations of Erastin (FIG. 9A) or a fixed concentration of Erastin (10 μM, FIG. 9B/C). In addition, 50,000 reporter THP1-dual cells were added to some plates (FIG. 9C). 24 hours later, HT1080 cell viability was measured (FIG. 9A/B) or THP1 supernatant was assessed for NFKB activity (FIG. 9C). HT1080 cells were acquired from ATCC and THP1-Dual cells were acquired from InvivoGen (San Diego, Calif.).

### Results:

The ACSL4 gene encodes Long-chain-fatty-acid-CoA ligase 4, an acyl-CoA synthetase that controls the level of arachidonic acid in cells, and is involved in the regulation of cell death. The CARS gene encodes cysteinyl-tRNA synthetase. Knockdown of CARS has been shown to inhibit erastin-induced ferroptosis by preventing the induction of lipid reactive oxygen species. See Hayano et al., 2016, Cell Death Differ. 23(2): 270-278. As shown in FIGS. 9A and 9B, genetic knockdown of either ACSL4 or CARS in HT1080 cells partially rescues viability of HT1080 cells cultured in the presence of Erastin. In addition, genetic knockdown of either ACSL4 or CARS in HT1080 cells abrogates NFKB activity in monocytes co-cultured with Erastin-treated HT1080 cells (FIG. 9C). These results demonstrate that cell death plays a role in the induction of NFKB signaling elicited by Erastin-treated HT1080 cells and that the absence of specific intracellular proteins reduces the pro-inflammatory nature of ferroptosis. In addition, these results demonstrate that agents that inhibit iron-dependent cellular disassembly (e.g. ferroptosis) can decrease immune activity (e.g. NFKB activity).

### Example 18: Specificity of Pro-Inflammatory Signaling Elicited by A20 Cells Treated with a GPX4 Inhibitor (RSL3, ML162 or ML210)

### Agent/Therapeutic Design:

A20 lymphoma cells were treated with various doses (e.g. 0.002, 0.005, 0.014, 0.041, 0.123, 0.370, 1.111, 3.333 and 10 μM) of a GPX4 inhibitor (RSL3, ML162 or ML210) in the presence or absence of 1 μM Ferrostatin-1 for 24 hours. ML162 was purchased from Cayman Chemical Company Inc and resuspended in DMSO. ML210 was purchased from Sigma-Aldrich and resuspended in DMSO. A20 lymphoma cells were also treated with DMSO as a negative control. After treatment with DMSO or a GPX4 inhibitor for 24 hours, the A20 lymphoma cells were co-cultured with THP1-Dual cells for an additional 24 hours. Subsequently, the THP1 supernatant was assessed for NFKB reporter activity. A20 cells were acquired from ATCC and THP1-Dual cells were acquired from InvivoGen (San Diego, Calif.). Both cell types were cultured in 96-well plates for the duration of the assay.

### Results:

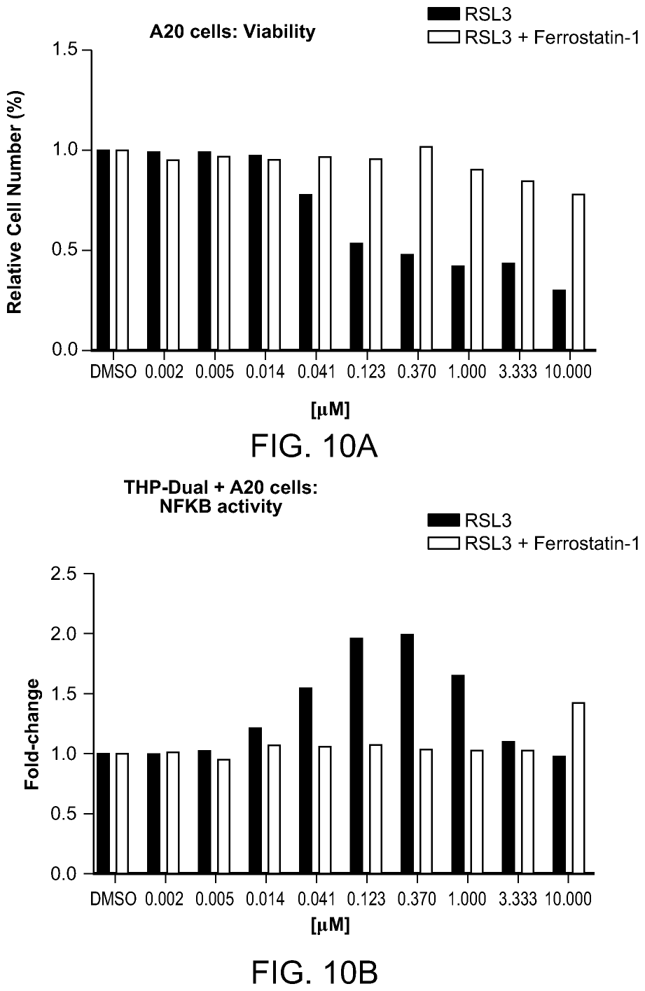

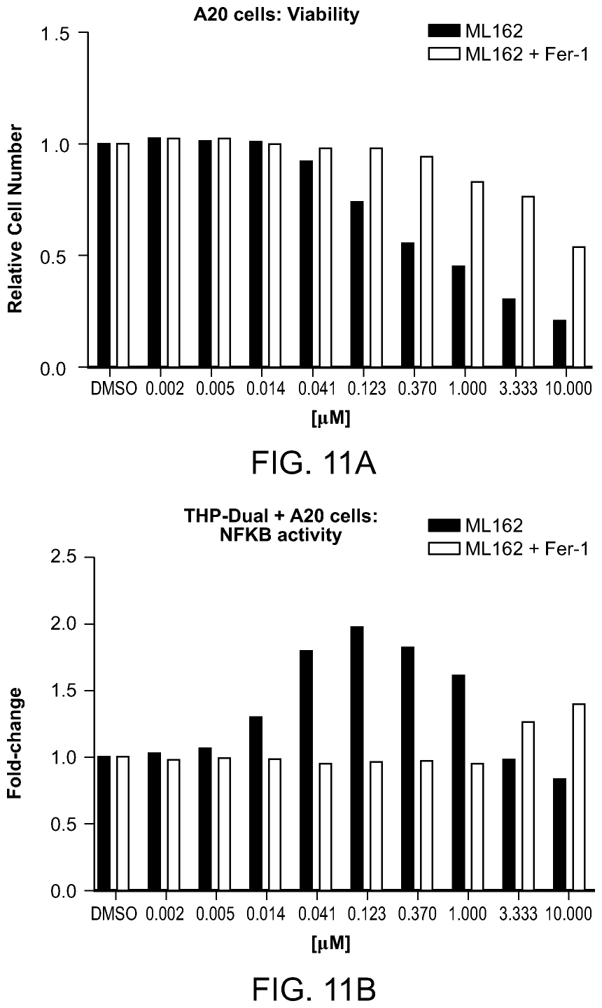

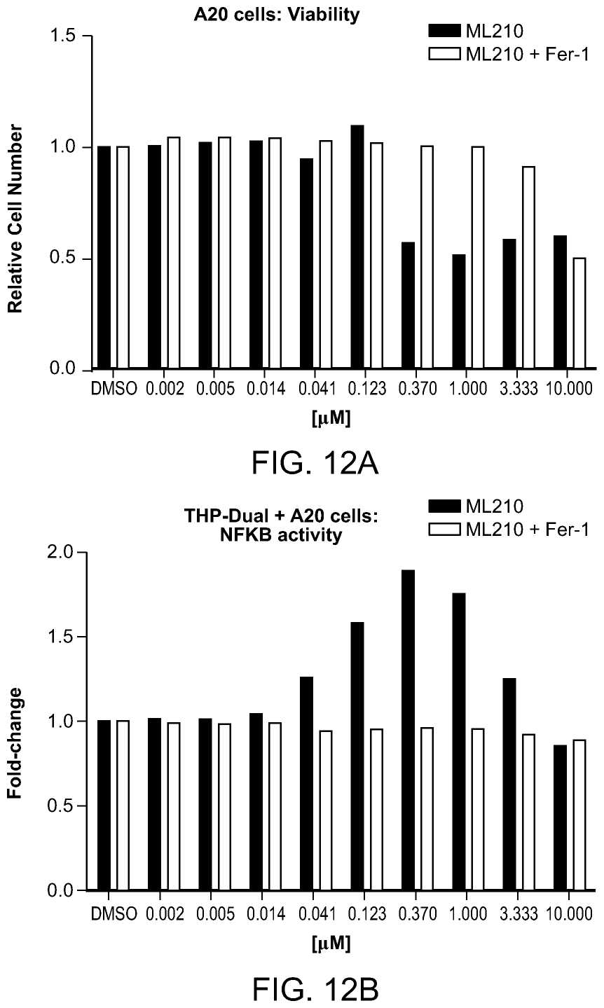

As discussed herein, GPX4 inhibitors such as RSL3, ML162 and ML210 can induce iron-dependent cellular disassembly (e.g. ferroptosis). As shown in FIGS. 10A, 11A and 12A, treatment of A20 lymphoma cells with each of the GPX4 inhibitors (RSL3, ML162 or ML210) decreased viability of the cells in a dose dependent manner, and this decreased viability was attenuated by the ferroptosis inhibitor Ferrostatin-1. As shown in FIGS. 10B, 11B and 12B, GPX4 inhibitor treatment of A20 lymphoma cells increased NFKB activity in THP1 cells in a dose dependent manner, and this increased NFKB activity was attenuated by the ferroptosis inhibitor Ferrostatin-1. These results demonstrate that cell death plays a role in the induction of NFKB signaling elicited by GPX-4 inhibitor-treated A20 lymphoma cells. In addition, because the ferroptosis inhibitor Ferrostatin-1 reduced NFKB activity induction by cells treated with a GPX4 inhibitor, these results demonstrate that inhibiting iron-dependent cellular disassembly (e.g., by a ferroptosis inhibitor) can decrease immune activity (e.g. NFKB activity).

### Example 19: Specificity of Pro-Inflammatory Signaling Elicited by Caki-1 Renal Carcinoma Cells Treated with a GPX4 Inhibitor (RSL3 or ML162)

### Agent/Therapeutic Design:

Caki-1 renal carcinoma cells were treated with either control (DMSO) or various doses (e.g. 0.002, 0.005, 0.014, 0.041, 0.123, 0.370, 1.111, 3.333 and 10 μM) of a GPX4 inhibitor (RSL3 or ML162) in the presence or absence of 1 μM Ferrostatin for 24 hours prior to co-culture with THP1-Dual cells for an additional 24 hours. Subsequently, the THP1 supernatant was assessed for NFKB reporter activity. Caki-1 cells were acquired from ATCC and THP1-Dual cells acquired from InvivoGen (San Diego, Calif.). Both cell types were cultured in 96-well plates for the duration of the assay.

### Results:

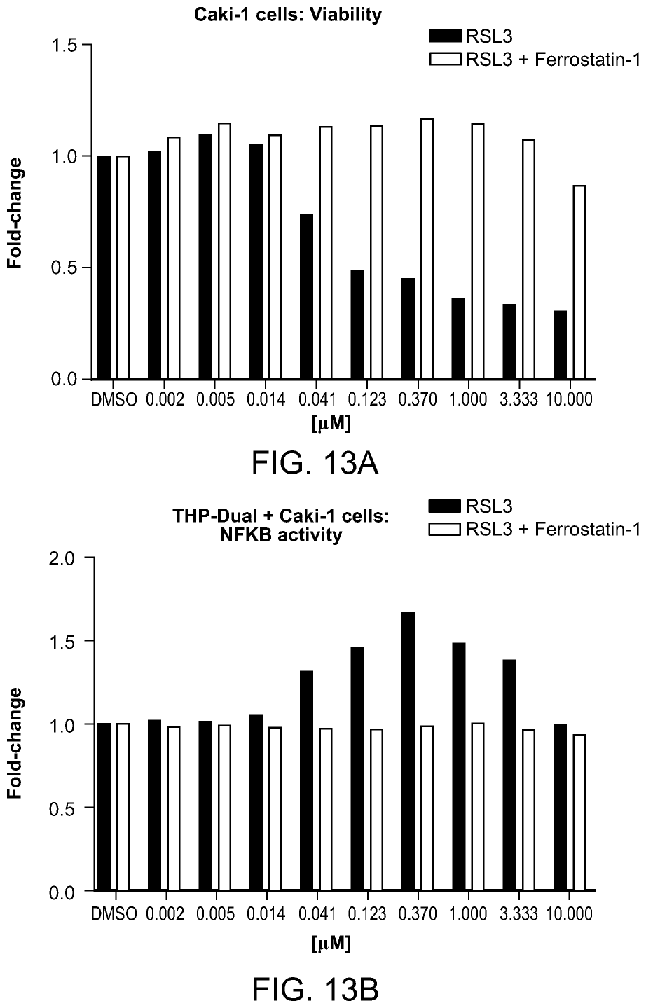

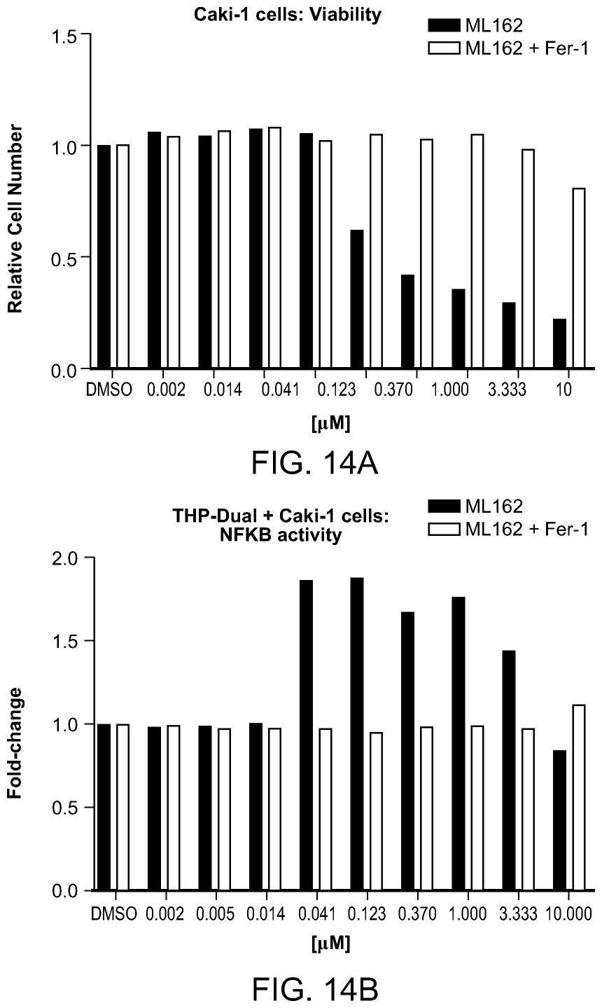

As shown in FIGS. 13A and 14A, treatment of Caki-1 renal carcinoma cells with a GPX4 inhibitor (RSL3 or ML162) decreased viability of the cells in a dose dependent manner, and this decreased viability was attenuated by the ferroptosis inhibitor Ferrostatin-1. As shown in FIGS. 13B and 14B, treatment of Caki-1 renal carcinoma cells with RSL3 or ML162 increased NFKB activity in THP1 cells in a dose dependent manner, and this increased NFKB activity was attenuated by the ferroptosis inhibitor Ferrostatin-1. These results demonstrate that cell death plays a role in the induction of NFKB signaling elicited by GPX-4 inhibitor-treated Caki-1 renal carcinoma cells. In addition, because the ferroptosis inhibitor Ferrostatin-1 reduced NFKB activity induction by cells treated with a GPX4 inhibitor, these results demonstrate that inhibiting iron-dependent cellular disassembly (e.g., by a ferroptosis inhibitor) can decrease immune activity (e.g. NFKB activity).

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

### INCORPORATION BY REFERENCE

Each reference, patent, and patent application referred to in the instant application is hereby incorporated by reference in its entirety as if each reference were noted to be incorporated individually.

1. A method of decreasing immune activity in a cell, tissue or subject, the method comprising administering to the cell, tissue or subject an agent that inhibits iron-dependent cellular disassembly in an amount sufficient to decrease the immune activity relative to a cell, tissue or subject that is not treated with the agent that inhibits iron-dependent cellular disassembly, wherein the agent that inhibits iron-dependent cellular disassembly comprises Ferrostatin-1 or a derivative or analog thereof.

2. The method of claim 1, wherein the Ferrostatin-1 derivative or analog thereof is represented by the following formula: 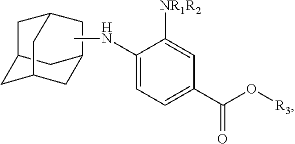
or an N-oxide, crystalline form, hydrate, or pharmaceutically acceptable salt thereof, wherein:
R₁ and R₂, are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, deuterium, C₁₋₄alkyl, CF₃, and combinations thereof; and
R₃ is independently selected from the group consisting of H, C₁₋₁₂aliphatic, C₁₋₆-alkyl-aryl and C₁₋₆-alkyl-heteroaryl;
with the proviso that:
when R₃ is ethyl, R₁ and R₂ cannot be both H, O, or 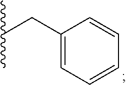 [SMILES: *Cc1ccccc1]
and
when R₃ is ethyl and at least one of R₁ or R₂ is H, R₁ or R₂ cannot be

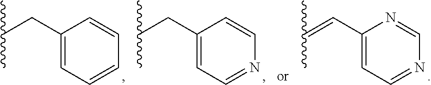

3. The method of claim 1, wherein the Ferrostatin-1 derivative or analog thereof is represented by the following formula: 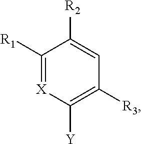
or a pharmaceutically acceptable salt, enantiomer, diastereomer thereof, wherein
X is CH or N;
Y is H, halo, or C₁₋₄alkyl;
R₁ is selected from the group consisting of H, halo, cycloalkyl, and NR₄R₅;
R₂ is selected from the group consisting of NR₆R₇ and NO2;
R₃ is selected from the group consisting of H, 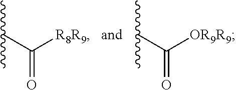
R₄ and R₅ are independently selected from the group consisting of H, C₁₋₁₂alkyl, C₃₋₁₂cycloalkyl, and aryl, wherein one or more of the ring carbons of the cycloalkyl are optionally substituted with one or more heteroatoms, and the cycloalkyl optionally comprises one or more pendant groups selected from the group consisting of H, F, NR₁₀R₁₁, Boc, COOR₁₂, and C₁₋₈alkyl;
R₆ and R₇ are independently selected from the group consisting of H, C₁₋₆alkyl, Boc, O, COOR₁₂, 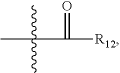
and C₁₋₃alkyl-aryl, wherein one or more of the ring carbons of the alkyl-aryl are optionally substituted with one or more nitrogen atoms, and the alkyl-aryl optionally comprises one or more pendant groups selected from the group consisting of H, halo, CN, NO₂, C₁₋₄ ether, C₁₋₄ ester, OCOOR₁₂, and C₁₋₈alkyl, which C₁₋₈alkyl is optionally further substituted with one or more halo;
R₈ and R₉ are independently selected from the group consisting of no atom, O, N, NHR₁₂, C₁₋₁₀ alkyl, and C₁₋₁₀ ether, wherein the alkyl and the ether are optionally substituted with NH₂, NHBOC, or C₃₋₁₂cycloalkyl, wherein one or more of the ring carbons of the cycloalkyl are optionally substituted with one or more heteroatoms;
R₁₀ and R₁₁ are independently selected from H and Boc; and
R₁₂ is a C₁₋₄alkyl optionally substituted with aryl,
with the proviso that:
when R₁ is H, R₃ cannot be 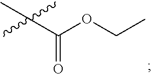 [SMILES: *C(=O)OCC]
when R₁ is 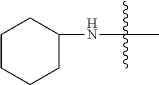 [SMILES: *NC1CCCCC1]
and R₂ is NH₂, R₃ cannot be 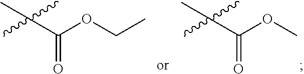
when R₁ is 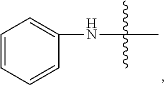 [SMILES: *Nc1ccccc1]
R₃ cannot be 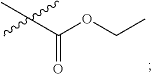 [SMILES: *C(=O)OCC]
when R₁ is 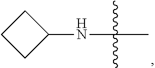 [SMILES: *NC1CCC1]
R₃ cannot be 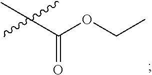 [SMILES: *C(=O)OCC]
when R₁ is Cl, X cannot be N, and
both R₁ and Y cannot be F.

4. The method of any one of claims 1 to 3, wherein the subject is in need of decreased immune activity.

5. The method of any one of claims 1 to 3, wherein the agent that inhibits iron-dependent cellular disassembly is administered in an amount sufficient to decrease in the cell, tissue or subject one or more of: the level or activity of NFkB, the level or activity of interferon regulatory factor (IRF) or Stimulator of Interferon Genes (STING), the level or activity of macrophages, the level or activity of monocytes, the level or activity of dendritic cells, the level or activity of T cells, the level or activity of CD4+, CD8+ or CD3+ cells, and the level or activity of a pro-immune cytokine.

6. A method of decreasing immune activity in a cell, tissue or subject, the method comprising administering to the cell, tissue or subject an agent that inhibits iron-dependent cellular disassembly in an amount sufficient to decrease the immune activity relative to a cell, tissue or subject that is not treated with the agent that inhibits iron-dependent cellular disassembly.

7. The method of claim 6, wherein the subject is in need of decreased immune activity.

8. The method of claim 6 or 7, wherein the agent that inhibits iron-dependent cellular disassembly is administered in an amount sufficient to decrease in the cell, tissue or subject one or more of: the level or activity of NFkB, the level or activity of interferon regulatory factor (IRF) or Stimulator of Interferon Genes (STING), the level or activity of macrophages, the level or activity of monocytes, the level or activity of dendritic cells, the level or activity of T cells, the level or activity of CD4+, CD8+ or CD3+ cells, and the level or activity of a pro-immune cytokine.

9. A method of decreasing the level or activity of NFkB in a cell, tissue or subject, comprising administering to the cell, tissue or subject an agent that inhibits iron-dependent cellular disassembly in an amount sufficient to decrease the level or activity of NFkB relative to a cell, tissue or subject that is not treated with the agent that inhibits iron-dependent cellular disassembly.

10. The method of claim 9, wherein the subject is in need of a decreased level or activity of NFkB.

11. The method of claim 9 or 10, wherein the level or activity of NFkB is decreased by at least 10%, 20%, 30%, 40%, 50%, 60%, 70%, 80%, 90% or 99%, or by at least 2-fold, 4-fold, 6-fold, 8-fold, or 10-fold relative to a cell, tissue or subject that is not treated with the agent that inhibits iron-dependent cellular disassembly.

12. A method of decreasing the level or activity of IRF or Stimulator of Interferon Genes (STING) in a cell, tissue or subject, comprising administering to the cell, tissue or subject an agent that inhibits iron-dependent cellular disassembly in an amount sufficient to decrease the level or activity of IRF or STING relative to a cell, tissue or subject that is not treated with the agent that inhibits iron-dependent cellular disassembly.

13. The method of claim 12, wherein the subject is in need of a decreased level or activity of IRF or STING.

14. The method of claim 12 or 13, wherein the level or activity of IRF or STING is increased by at least 10%, 20%, 30%, 40%, 50%, 60%, 70%, 80%, 90% or 99%, or by at least 2-fold, 4-fold, 6-fold, 8-fold, or 10-fold relative to a cell, tissue or subject that is not treated with the agent that inhibits iron-dependent cellular disassembly.

15. A method of decreasing the level or activity of macrophages, monocytes, dendritic cells or T cells in a tissue or subject, comprising administering to the tissue or subject an agent that inhibits iron-dependent cellular disassembly in an amount sufficient to increase the level or activity of macrophages, monocytes, dendritic cells or T cells relative to a tissue or subject that is not treated with the agent that inhibits iron-dependent cellular disassembly.

16. The method of claim 15, wherein the subject is in need of a decreased level or activity of macrophages, monocytes, dendritic cells or T cells.

17. The method of claim 15 or 16, wherein the level or activity of macrophages, monocytes, dendritic cells, or T cells is decreased by at least 10%, 20%, 30%, 40%, 50%, 60%, 70%, 80%, 90% or 99%, or by at least 2-fold, 4-fold, 6-fold, 8-fold, or 10-fold relative to a tissue or subject that is not treated with the agent that inhibits iron-dependent cellular disassembly.

18. A method of decreasing the level or activity of CD4+, CD8+, or CD3+ cells in a tissue or subject, comprising administering to the subject an agent that inhibits iron-dependent cellular disassembly in an amount sufficient to decrease the level or activity of CD4+, CD8+, or CD3+ cells relative to a tissue or subject that is not treated with the agent that inhibits iron-dependent cellular disassembly.

19. The method of claim 18, wherein the subject is in need of a decreased level or activity of CD4+, CD8+, or CD3+ cells.

20. The method of claim 18 or 19, wherein the level or activity of CD4+, CD8+, or CD3+ cells is decreased by at least 10%, 20%, 30%, 40%, 50%, 60%, 70%, 80%, 90% or 99%, or by at least 2-fold, 4-fold, 6-fold, 8-fold, or 10-fold relative to a tissue or subject that is not treated with the agent that inhibits iron-dependent cellular disassembly.

21. A method of decreasing the level or activity of a pro-immune cytokine in a cell, tissue or subject, comprising administering to the cell, tissue or subject an agent that inhibits iron-dependent cellular disassembly in an amount sufficient to decrease the level or activity of the pro-immune cytokine relative to a cell, tissue or subject that is not treated with the agent that inhibits iron-dependent cellular disassembly.

22. The method of claim 21, wherein the subject is in need of a decreased level or activity of a pro-immune cytokine.

23. The method of claim 21 or 22, wherein the level or activity of the pro-immune cytokine is decreased by at least 10%, 20%, 30%, 40%, 50%, 60%, 70%, 80%, 90% or 99%, or by at least 2-fold, 4-fold, 6-fold, 8-fold, or 10-fold relative to a cell, tissue or subject that is not treated with the agent that inhibits iron-dependent cellular disassembly.

24. The method of claim 22 or 23, wherein the pro-immune cytokine is selected from IFN-α, IL-1, IL-12, IL-18, IL-2, IL-15, IL-4, IL-6, TNF-α, IL-17 and GMCSF.

25. The method of any one of claims 1 to 24, further comprising, before the administering, evaluating the cell, tissue or subject for one or more of: the level or activity of NFkB; the level or activity of macrophages; the level or activity of monocytes; the level or activity of dendritic cells; the level or activity of CD4+ cells, CD8+ cells, or CD3+ cells; the level or activity of T cells; and the level or activity of a pro-immune cytokine.

26. The method of any one of claims 1 to 24, further comprising, after the administering, evaluating the cell, tissue or subject for one or more of: the level or activity of NFkB; the level or activity of macrophages; the level or activity of monocytes; the level or activity of dendritic cells; the level or activity of CD4+ cells, CD8+ cells or CD3+ cells; the level or activity of T cells; and the level or activity of a pro-immune cytokine.

27. The method of any one of claims 21 to 26, wherein the pro-immune cytokine is selected from IFN-α, IL-1, IL-12, IL-18, IL-2, IL-15, IL-4, IL-6, TNF-α, IL-17 and GMCSF.

28. A method of treating a subject in need of decreased immune activity, the method comprising administering to the subject an agent that inhibits iron-dependent cellular disassembly in an amount sufficient to decrease the immune activity in the subject.

29. The method of claim 28, wherein the subject has a disorder associated with iron-dependent cellular disassembly.

30. The method of claim 28, wherein the subject has a disorder in which iron-dependent cellular disassembly is detrimental.

31. The method of any one of claims 28 to 30, wherein the method further comprises evaluating the subject for iron-dependent cellular disassembly.

32. The method of any one of claims 1 to 31, wherein the subject has an inflammatory disease or condition.

33. The method of claim 32, wherein the inflammatory disease or condition is selected from the group consisting of inflammation, acute organ injury, tissue damage, sepsis, atherosclerosis, a neurodegenerative disorder, and an immune-related disease or condition.

34. The method of claim 33, wherein the inflammation is selected from sterile inflammation, chronic inflammation, and acute inflammation in response to disease or injury.

35. The method of claim 33, wherein the immune-related disease or condition is an autoimmune disease.

36. The method of claim 35, wherein the autoimmune disease is selected from systemic lupus erythematosus (SLE), rheumatoid arthritis, Type I diabetes, Type II diabetes, multiple sclerosis (MS), amyotrophic lateral sclerosis (ALS), graft-vs-host disease (GVHD), psoriasis, and ulcerative colitis.

37. The method of claim 33, wherein the immune-related disease or condition is an allergy or asthma.

38. The method of claim 33, wherein the immune-related disease or condition is an autoinflammatory condition.

39. The method of any one of claims 6 to 38, wherein the agent that inhibits iron-dependent cellular disassembly is selected from an inhibitor of lipid peroxidation, an inhibitor of glutaminolysis, an inhibitor of lipoxygenase, an inhibitor of cysteine dioxygenase 1 (CDO1), and an inhibitor of DPP4.

40. The method of any one of claims 6 to 38, wherein the agent that inhibits iron-dependent cellular disassembly is selected from cycloheximide, beta-mercaptoethanol, dopamine, and selenium.

41. The method of claim 39, wherein the inhibitor of lipoxygenase is an inhibitor of arachidonate lipoxygenase (ALOX).

42. The method of claim 39, wherein the inhibitor of arachidonate lipoxygenase is selected from the group consisting of CDC, baicalein, PD-146176, AA-861, and zileuton.

43. The method of claim 39, wherein the inhibitor of lipid peroxidation is selected from vitamin E, alpha-tocopherol, trolox, tocotrienols, deuterated polyunsaturated fatty acids (D-PUFAs), butylated hydroxytoluene, butylated hydroxyanisole, Ferrostatin-1 or a derivative or analog thereof, Fiproxstatin-1 or a derivative or analog thereof, Coenzyme Q10, idebenone, XJB-5-131, deferoxamine, cyclipirox, and deferiprone.

44. The method of any one of claims 6 to 38, wherein the agent that inhibits iron-dependent cellular disassembly is Ferrostatin-1 or a derivative or analog thereof.

45. The method of claim 44, wherein the Ferrostatin-1 derivative or analog thereof is represented by the following formula: 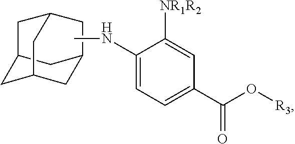
or an N-oxide, crystalline form, hydrate, or pharmaceutically acceptable salt thereof, wherein:
R₁ and R₂, are independently selected from the group consisting of no atom, H, D, O, halo, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, deuterium, C₁₋₄alkyl, CF₃, and combinations thereof; and
R₃ is independently selected from the group consisting of H, C₁₋₁₂aliphatic, C₁₋₆-alkyl-aryl and C₁₋₆-alkyl-heteroaryl;
with the proviso that:
when R₃ is ethyl, R₁ and R₂ cannot be both H, O, or 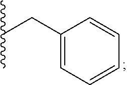 [SMILES: *Cc1ccccc1]
and
when R₃ is ethyl and at least one of R₁ or R₂ is H, R₁ or R₂ cannot be

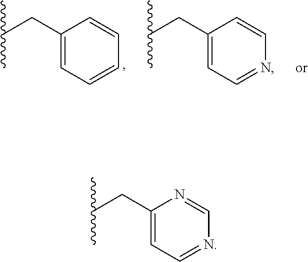

46. The method of claim 44, wherein the Ferrostatin-1 derivative or analog thereof is represented by the following formula: 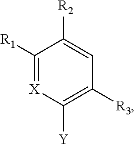
or a pharmaceutically acceptable salt, enantiomer, diastereomer thereof, wherein
X is CH or N;
Y is H, halo, or C₁₋₄alkyl;
R₁ is selected from the group consisting of H, halo, cycloalkyl, and NR₄R₅;
R₂ is selected from the group consisting of NR₆R₇ and NO2;
R₃ is selected from the group consisting of H, 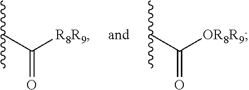
R₄ and R₅ are independently selected from the group consisting of H, C₁₋₁₂alkyl, C₃₋₁₂cycloalkyl, and aryl, wherein one or more of the ring carbons of the cycloalkyl are optionally substituted with one or more heteroatoms, and the cycloalkyl optionally comprises one or more pendant groups selected from the group consisting of H, F, NR₁₀R₁₁, Boc, COOR₁₂, and C₁₋₈alkyl;
R₆ and R₇ are independently selected from the group consisting of H, C₁₋₆alkyl, Boc, O, COOR₁₂, 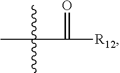
and C₁₋₃alkyl-aryl, wherein one or more of the ring carbons of the alkyl-aryl are optionally substituted with one or more nitrogen atoms, and the alkyl-aryl optionally comprises one or more pendant groups selected from the group consisting of H, halo, CN, NO₂, CM ether, C₁₋₄ ester, OCOOR₁₂, and C₁₋₈alkyl, which C₁₋₈alkyl is optionally further substituted with one or more halo;
R₈ and R₉ are independently selected from the group consisting of no atom, O, N, NHR₁₂, C₁₋₁₀ alkyl, and C₁₋₁₀ ether, wherein the alkyl and the ether are optionally substituted with NH₂, NHBOC, or C₃₋₁₂cycloalkyl, wherein one or more of the ring carbons of the cycloalkyl are optionally substituted with one or more heteroatoms;
R₁₀ and R₁₁ are independently selected from H and Boc; and
R₁₂ is a C₁₋₄alkyl optionally substituted with aryl,
with the proviso that:
when R₁ is H, R₃ cannot be 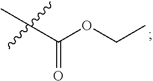 [SMILES: *C(=O)OCC]
when R₁ is 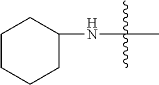 [SMILES: *NC1CCCCC1]
and R₂ is NH₂, R₃ cannot be 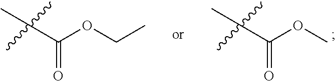
when R₁ is 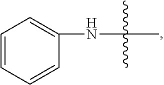 [SMILES: *Nc1ccccc1]
R₃ cannot be 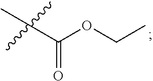 [SMILES: *C(=O)OCC]
when R₁ is 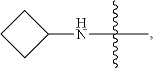 [SMILES: *NC1CCC1]
R₃ cannot be 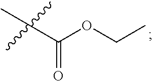 [SMILES: *C(=O)OCC]
when R₁ is Cl, X cannot be N, and
both R₁ and Y cannot be F.

47. The method of claim 39, wherein the inhibitor of DPP4 is selected from vildagliptin, alogliptin, and linagliptin.

48. The method of any one of claims 6 to 38, wherein the agent that inhibits iron-dependent cellular disassembly is 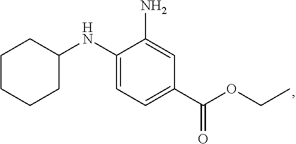 [SMILES: CCOC(=O)c1ccc(NC2CCCCC2)c(N)c1]
or a pharmaceutically acceptable salt thereof.

49. The method of any one of claims 6 to 38, wherein the agent that inhibits iron-dependent cellular disassembly is 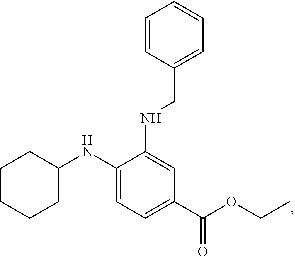 [SMILES: CCOC(=O)c1ccc(NC2CCCCC2)c(NCc2ccccc2)c1]
or a pharmaceutically acceptable salt thereof.

50. The method of any one of claims 6 to 38, wherein the agent that inhibits iron-dependent cellular disassembly is 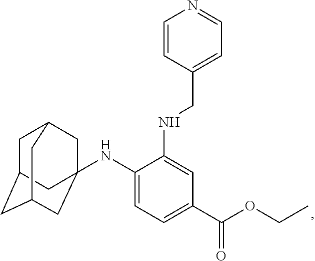 [SMILES: CCOC(=O)c1ccc(NC23CC4CC(CC(C4)C2)C3)c(NCc2ccncc2)c1]
or a pharmaceutically acceptable salt thereof.

51. A method of screening for an immunoinhibitory agent, the method comprising:
(a) providing a plurality of test agents;
(b) evaluating each of the plurality of test agents for the ability to inhibit iron-dependent cellular disassembly;
(c) selecting as a candidate immunoinhibitory agent a test agent that inhibits iron-dependent cellular disassembly; and
(d) evaluating the candidate immunoinhibitory agent for the ability to decrease an immune response.

52. The method of claim 51, wherein the evaluating step (b) comprises contacting cells, tissue, or a subject with each of the plurality of test agents.

53. The method of claim 51, wherein the evaluating step (b) comprises contacting cells, tissue, or a subject with an agent that induces iron-dependent cellular disassembly.

54. The method of claim 52, wherein the subject is an animal.

55. The method of claim 51, wherein the evaluating step (b) further comprises measuring the level or activity of a marker selected from the group consisting of lipid peroxidation, reactive oxygen species (ROS), isoprostanes, malondialdehyde (MDA), iron, glutathione peroxidase 4 (GPX4), prostaglandin-endoperoxide synthase 2 (PTGS2), cyclooxygenase-2 (COX-2), and glutathione (GSH) in the cells or tissue contacted with the test agent.

56. The method of claim 51, wherein the evaluating step (b) further comprises comparing the level or activity of the marker in the cells, tissue or subject contacted with the test agent to the level or activity of the marker in a control cell, control tissue or control subject that has not been contacted with the test agent.

57. The method of claim 56, wherein the control cell, control tissue or control subject has been contacted with an agent that induces iron-dependent cellular disassembly.

58. The method of any one of claims 51 to 57, wherein a decrease in the level or activity of a marker selected from the group consisting of lipid peroxidation, isoprostanes, reactive oxygen species (ROS), iron, PTGS2 and COX-2 in the cells, tissue or subject contacted with the test agent relative to the level or activity of the marker in a control cell, control tissue or control subject that has not been contacted with the test agent indicates that the test agent is an agent that inhibits iron-dependent cellular disassembly.

59. The method of any one of claims 51 to 57, wherein an increase in the level or activity of a marker selected from the group consisting of GPX4, MDA and GSH in the cells, tissue or subject contacted with the test agent relative to the level or activity of the marker in a control cell, control tissue or control subject that has not been contacted with the test agent indicates that the test agent is an agent that inhibits iron-dependent cellular disassembly.

60. The method of claim 51, wherein evaluating the candidate immunoinhibitory agent comprises culturing an immune cell together with cells contacted with the selected candidate immunoinhibitory agent or exposing an immune cell to postcellular signaling factors produced by cells contacted with the selected candidate immunoinhibitory agent and measuring the level or activity of NFκB, IRF or STING in the immune cell.

61. The method of claim 60, wherein the immune cell is a THP-1 cell.

62. The method of claim 60, wherein evaluating the candidate immunoinhibitory agent comprises culturing T cells together with cells contacted with the selected candidate immunoinhibitory agent or exposing T cells to postcellular signaling factors produced by cells contacted with the selected candidate immunoinhibitory agent and measuring the activation and proliferation of the T cells.

63. The method of claim 60, wherein evaluating the candidate immunoinhibitory agent further comprises comparing the level or activity of NFκB, IRF or STING in the immune cell cultured with cells contacted with the selected candidate immunoinhibitory agent or exposed to postcellular signaling factors produced by cells contacted with the selected candidate immunoinhibitory agent with the level or activity of NFκB, IRF or STING in control immune cells that have not been contacted with the selected candidate immunoinhibitory agent and have not been exposed to postcellular signaling factors produced by cells contacted with the selected candidate immunoinhibitory agent.

64. The method of claim 63, wherein the cells contacted with the selected candidate immunoinhibitory agent have also been contacted with an agent that induces iron-dependent cellular disassembly.

65. The method of any one of claims 53, 57 and 64, wherein the agent that induces iron-dependent cellular disassembly is selected from the group consisting of an inhibitor of antiporter system Xc⁻, an inhibitor of GPX4, and a statin.

66. The method of claim 65, wherein the inhibitor of antiporter system Xc⁻ is erastin or a derivative or analog thereof.

67. The method of claim 66, wherein the analog of erastin is PE or IKE.

68. The method of claim 65, wherein the inhibitor of GPX4 is selected from the group consisting of (1S,3R)-RSL3 or a derivative or analog thereof, ML162, DPI compound 7, DPI compound 10, DPI compound 12, DPI compound 13, DPI compound 17, DPI compound 18, DPI compound 19, FIN56, and FINO2.

69. The method of claim 65, wherein the statin is selected from the group consisting of atorvastatin, fluvastatin, lovastatin, pitavastatin, pravastatin, rosuvastatin, cerivastatin and simvastatin.

70. The method of any one of claims 53, 57 and 64, wherein the agent that induces iron-dependent cellular disassembly is selected from the group consisting of sorafenib or a derivative or analog thereof, sulfasalazine, glutamate, BSO, DPI2, cisplatin, cysteinase, silica based nanoparticles, CCI4, ferric ammonium citrate, trigonelline and brusatol.
